(19) United States
(12) Patent Application Publication
(10) Pub. No.: US 2025/0266124 A1
(43) Pub. Date: Aug. 21, 2025

(54) SYSTEMS AND METHODS FOR DETERMINING ANTIGEN SPECIFICITY OF ANTIGEN BINDING MOLECULES AND VISUALIZING ADAPTIVE IMMUNE CELL CLONOTYPING DATA

(71) Applicant: 10x Genomics, Inc., Pleasanton, CA (US)

(72) Inventors: Vartika Agrawal, Pleasanton, CA (US); Michael John Terry Stubbington, 94588-3260, CA (US); David Benjamin Jaffe, Pleasanton, CA (US); Wyatt James McDonnell, Pleasanton, CA (US); Peigeng Li, Pleasanton, CA (US); Jessica Hamel, Pleasanton, CA (US); Brett Olsen, Pleasanton, CA (US); Du Linh Lam, Pleasanton, CA (US); Guy Joseph, Pleasanton, CA (US); Nur-Taz Rahman, Pleasanton, CA (US); Didem Pelin Sarikaya, Pleasanton, CA (US)

(21) Appl. No.: 19/197,069

(22) Filed: May 2, 2025

Related U.S. Application Data
(63) Continuation of application No. PCT/US23/78758, filed on Nov. 4, 2023.
(60) Provisional application No. 63/422,871, filed on Nov. 4, 2022.

(51) Int. Cl. G16B 15/30; G16B 45/00

(52) U.S. Cl. CPC G16B 15/30; G16B 45/00

(57) ABSTRACT

Provided herein are computer-implemented methods including receiving, by a processor, a data set comprising cellular data, and presenting an end user with a visualization tool, wherein the cellular data may include at least one of an antigen binding specificity value associated with a cell receptor and a unique molecular identifier (UMI) count associated with the cell receptor. The visualization tool can provide a dynamic display of the data set by generating a visual sequence from the data set, displaying at least a portion of the generated visual sequence, and displaying in response to a user interaction with the visualization tool, the antigen binding specificity value or the UMI associated with the cell receptor in conjunction with the at least a portion of the visual sequence. The visual sequences include sets of indicia corresponding paired chains of the cell receptor.

### CROSS-REFERENCE TO RELATED APPLICATIONS

This application claims the benefit of U.S. Provisional Application No. 63/422,871, filed Nov. 4, 2022, which is hereby incorporated by reference in its entirety.

### FIELD

This description is generally directed towards systems and methods for analyzing immune cell clonotype data generated using single- and multi-modal single cell nucleic acid sequencing technologies. More specifically, there is a need for systems and methods to assess the antigen specificity of antigen binding molecules. There is also a need for systems and methods to visualize and present immune cell clonotype data so that it is readily analyzed and interpreted by a user. Systems and methods to assess, visualize, and present these data for analysis and interpretation are useful and readily applied to data generated using non-droplet and droplet-based single cell nucleic acid sequencing technologies, array-based microwell- and nanowell-based single cell nucleic acid sequencing technologies, in situ sequencing technologies, and spatially indexed single cell technologies.

### BACKGROUND

The immune system recognizes and eliminates non-self threats through a complex and layered network of both innate and adaptive immune cells. Robust characterization of this response and discovery of novel cell types and antigen-specific populations has proven challenging to perform in a high-throughput fashion due to the limited number of analytes that can be measured simultaneously using flow cytometry, CyTOF, and similar assays. One approach to addressing these limitations is to utilize multi-modal single cell technologies, such as droplet-based single cell techniques. Applications of these technologies include the analysis of T cells, B cells, and peripheral blood mononuclear cells, e.g., pre- and post-vaccination samples, e.g., from influenza vaccines or other vaccines (or of samples collected from individuals affected by diseases such as systemic lupus erythematosus and other autoimmune disorders, chronic viral infection, and acute/non-chronic viral infection), or T cells/B cells/PBMCs from individuals treated with a drug or biological molecule such as a checkpoint inhibitor, anti-cancer drug, monoclonal antibody, or antibody-drug conjugate. Importantly, these single cell assays allow users to learn the full and paired sequences of heterodimeric and extremely polymorphic immune cell receptors of adaptive lymphocytes, e.g., T cells and B cells, and to identify from which single cell (and its corresponding phenotype, genotype, and antigen specificity) a given immune receptor had originated. This relationship is masked or not directly observable using bulk DNA and RNA-based sequencing assays and is not captured in a cost-effective or high-throughput fashion in plate-based assays.

Using this framework, T cell and B cell responses can be identified and used to implement an immune cell (B cells/T cells/PBMCs) clonotyping algorithm that immune receptor lineages at scale by combining untargeted and targeted gene expression, full-length immune cell receptor sequencing, surface protein expression and/or antigen capture, in addition to tag-based and genetic demultiplexing.

Additionally, the antigen receptors expressed by immune cells, such as B lymphocytes (or B-cells) and T lymphocytes (or T-cells), include two different polypeptide chains (e.g., heavy chain and light chain for B-cells and alpha chain and beta chain for T-cells). Each polypeptide chain of a receptor may include three complementarity determining regions. These (CDRs), which alternate with the framework regions (FRs) of the receptor.

complementarity determining regions (CDRs) are part of the variable chains of an antigen receptor that binds to a specific antigen. Thus, the antigen specificity of an antigen binding molecule (ABM), such as the antigen receptor of an immune cell, may be largely determined by the complementarity determining regions (CDRs) of the immune cell receptor. Identifying antibody binding molecules that binds to a target antigen with high affinity and sufficient specificity and that neutralizes the target antigen may be crucial for disease prevention and treatment. Nevertheless, identifying antigen binding molecules with sufficient binding specificity towards a target antigen may be time consuming and resource intensive endeavor.

As such, there is a need for systems and methods that can aid in the visualization, and presentation of immune cell clonotype data generated using single- and multi-modal single cell nucleic acid sequencing technologies for analysis and interpretation. There is also a need for system and methods for assessing antigen specificity that complement high throughput discovery of antibodies and T cell receptors, and aiding in visualization and presentation of such antigen specificity.

### SUMMARY

In one aspect, a computer implemented method for visualizing cellular data is disclosed. The method can comprise receiving, by a processor, a data set comprising cellular data, wherein the cellular data includes at least one of an antigen binding specificity value associated with a first cell receptor and a unique molecular identifier (UMI) count associated with the first cell receptor. The method can further comprise presenting an end user with a visualization tool. The visualization tool can provide a dynamic display of the data set by generating, by the processor, a first visual sequence from the data set, displaying at least a portion of the generated first visual sequence, and displaying in response to a user interaction with the visualization tool, the antigen binding specificity value or the UMI associated with the cell receptor in conjunction with the at least a portion of the first visual sequence. The first visual sequence includes a set of first indicia corresponding to a first chain of the first cell receptor and a set of second indicia corresponding to a second chain of the first cell receptor, wherein the set of first indicia is adjacent to the set of second indicia. The dynamic display of the data set can provide for analysis of the cellular data from the data set.

In another aspect, a computer implemented method for visualizing cellular data is disclosed. The method can comprise receiving, by a processor, a data set comprising cellular data. The method can further comprise receiving, by the processor, a selection of a filter, wherein the filter is selected from the group consisting of: UMI Counts/antigen, antigen specificity, barcode, number of barcodes representing a clonotype, CDR3 amino acid sequence, CDR3 bases, gene name, isotype, cluster, amino acid % mutation, nucleotide % mutation, INKT/MAIT, and combinations thereof. The method can further comprise presenting an end user with a visualization tool. The visualization tool can provide a dynamic display of the data set by generating, by the processor, a first visual sequence from the data set and, displaying at least a portion of the generated first visual sequence, generating, by the processor, a first table of information from the data set and displaying the first table of information, modifying, by the processor, the first visual sequence and the first table of information based on the filter, to generate a modified first visual sequence and modified first table of information that is different from the first visual sequence and the first table of information, and displaying the modified first visual sequence and modified first table of information. The first visual sequence includes a set of first indicia corresponding to a first chain of a first cell receptor and a set of second indicia corresponding to a second chain of the first cell receptor, wherein the set of first indicia is adjacent to the set of second indicia in a first row. The first table of information includes genetic information for at least one of identified clonotypes and cell receptors belonging to a clonotype that are presented in the first visual sequence. The dynamic display of the data set can provide for analysis of the cellular data from the data set.

In another aspect, a computer implemented method for visualizing cellular data is disclosed. The method can comprise receiving, by a first processor, a plurality of discrete data sets from one or more data sources; generating, by the first processor, a multi-section data file that combines the plurality of discrete data sets, wherein the multi-section data file includes cellular data including at least one of an antigen binding specificity value associated with a first cell receptor and a unique molecular identifier (UMI) associated with the first cell receptor; receiving, by a second processor, the multi-section data file; and presenting an end user with a visualization tool. The visualization tool can provide a dynamic display of the multi-section data file by generating, by a second processor, a first visual sequence from the multi-section data file, displaying at least a portion of the generated first visual sequence, and displaying in response to a user interaction with the visualization tool, the antigen binding specificity value or the UMI associated with the cell receptor in conjunction with the at least a portion of the first visual sequence. The first visual sequence includes a set of first indicia corresponding to a first chain of the first cell receptor and a set of second indicia corresponding to a second chain of the first cell receptor, wherein the set of first indicia is adjacent to the set of second indicia in a first row. The dynamic display of the multi-section data file can provide for analysis of the cellular data from the multi-section data file.

In another aspect, a computer implemented method for visualizing cellular data is disclosed. The method can comprise receiving, by a first processor, a plurality of discrete data sets from one or more data sources; generating, by the first processor, a multi-section data file that combines the plurality of discrete data sets, wherein the multi-section data file includes cellular data including at least one of an antigen binding specificity value associated with a first cell receptor and a unique molecular identifier (UMI) associated with the first cell receptor; receiving, by a second processor, the multi-section data file; and presenting an end user with a visualization tool. The visualization tool can provide a dynamic display of the multi-section data file by generating, by a second processor, a first visual sequence from the multi-section data file and, displaying at least a portion of the generated first visual sequence, generating, by the second processor, a first table of information from the multi-section data file and displaying the first table of information, modifying, by the second processor, the first visual sequence and the first table of information based on the filter, to generate a modified first visual sequence and modified first table of information that is different from the first visual sequence and the first table of information, and displaying the modified first visual sequence and modified first table of information. The first visual sequence includes a set of first indicia corresponding to a first chain of the first cell receptor and a set of second indicia corresponding to a second chain of the first cell receptor, wherein the set of first indicia is adjacent to the set of second indicia, e.g., in a first row. The first table of information includes genetic information for at least one of identified clonotypes and cell receptors belonging to a clonotype that are presented in the first visual sequence. The dynamic display of the multi-section data file can provide for analysis of the cellular data from the multi-section data file.

In another aspect, a system for visualizing cellular data is disclosed. The system includes a memory and a processor in communication with the memory. The processor is configured to perform the operations comprising receiving a data set comprising cellular data, wherein the cellular data includes at least one of an antigen binding specificity value associated with a first cell receptor and a unique molecular identifier (UMI) count associated with the first cell receptor. The operations can further comprise presenting an end user with a visualization tool. The visualization tool can provide a dynamic display of the data set via additional operations that the processor is configured to perform comprising generating a first visual sequence from the data set, displaying, at least a portion of the generated first visual sequence, and displaying, in response to a user interaction with the visualization tool, the antigen binding specificity value or the UMI associated with the cell receptor in conjunction with the at least a portion of the first visual sequence. The first visual sequence includes a set of first indicia corresponding to a first chain of the first cell receptor and a set of second indicia corresponding to a second chain of the first cell receptor, wherein the set of first indicia is adjacent to the set of second indicia in a first row. The dynamic display of the data set can provide for analysis of the cellular data from the data set.

In another aspect, a system for visualizing cellular data is disclosed. The system includes a memory and a processor in communication with the memory. The processor is configured to perform the operations comprising receiving, a data set comprising cellular data. The operations can further comprise receiving, a selection of a filter, wherein the filter is selected from the group consisting of: UMI Counts/antigen, antigen specificity, barcode, number of barcodes representing a clonotype, CDR3 amino acid sequence, CDR3 bases, gene name, isotype, cluster, amino acid % mutation, nucleotide % mutation, iNKT/MAIT, and combinations thereof. The operations can further comprise presenting an end user with a visualization tool. The visualization tool can provide a dynamic display of the data set via additional operations that the processor is configured to perform comprising generating, a first visual sequence from the data set and, displaying, at least a portion of the generated first visual sequence, generating, a first table of information from the data set and displaying, the first table of information, modifying, the first visual sequence and the first table of information based on the filter, to generate a modified first visual sequence and modified first table of information that is different from the first visual sequence and the first table of information, and displaying, the modified first visual sequence and modified first table of information. The first visual sequence includes a set of first indicia corresponding to a first chain of the first cell receptor and a set of second indicia corresponding to a second chain of the first cell receptor, wherein the set of first indicia is adjacent to the set of second indicia in a first row. The first table of information includes genetic information for at least one of identified clonotypes and cell receptors belonging to a clonotype that are presented in the first visual sequence. The dynamic display of the data set can provide for analysis of the cellular data from the data set.

In another aspect, a system for visualizing cellular data is disclosed. The system includes a first memory and a first processor in communication with the first memory, and a second memory and a second processor in communication with the second memory. The first processor is configured to perform first operations comprising receiving a plurality of discrete data sets from one or more data sources. The first operations further comprise generating a multi-section data file that combines the plurality of discrete data sets, wherein the multi-section data file includes cellular data including at least one of an antigen binding specificity value associated with a first cell receptor and a unique molecular identifier (UMI) associated with the first cell receptor. The second processor is configured to perform second operations comprising receiving the multi-section data file, and presenting an end user with a visualization tool. The visualization tool can provide a dynamic display of the multi-section data file via additional second operations that the second processor is configured to perform comprising generating a first visual sequence from the multi-section data file and, displaying at least a portion of the generated first visual sequence, generating a first table of information from the multi-section data file and displaying the first table of information, modifying the first visual sequence and the first table of information based on the filter, to generate a modified first visual sequence and modified first table of information that is different from the first visual sequence and the first table of information, and displaying the modified first visual sequence and modified first table of information. The first visual sequence includes a set of first indicia corresponding to a first chain of the first cell receptor and a set of second indicia corresponding to a second chain of the first cell receptor, wherein the set of first indicia is adjacent to the set of second indicia in a first row. The first table of information includes genetic information for at least one of identified clonotypes and cell receptors belonging to a clonotype that are presented in the first visual sequence. The dynamic display of the multi-section data file can provide for analysis of the cellular data from the multi-section data file.

In another aspect, a system for visualizing cellular data is disclosed. The system includes a first memory and a first processor in communication with the first memory, and a second memory and a second processor in communication with the second memory. The first processor is configured to perform first operations comprising receiving a plurality of discrete data sets from one or more data sources, and generating a multi-section data file that combines the plurality of discrete data sets, wherein the multi-section data file includes cellular data including at least one of an antigen binding specificity value associated with a first cell receptor and a unique molecular identifier (UMI) associated with the first cell receptor. The second processor is configured to perform second operations comprising receiving the multi-section data file, and presenting an end user with a visualization tool. The visualization tool can provide a dynamic display of the multi-section data file via additional second operations that the second processor is configured to perform comprising generating a first visual sequence from the multi-section data file and, displaying at least a portion of the generated first visual sequence, generating a first table of information from the multi-section data file and displaying the first table of information, modifying the first visual sequence and the first table of information based on the filter, to generate a modified first visual sequence and modified first table of information that is different from the first visual sequence and the first table of information, and displaying the modified first visual sequence and modified first table of information. The first visual sequence includes a set of first indicia corresponding to a first chain of the first cell receptor and a set of second indicia corresponding to a second chain of the first cell receptor, wherein the set of first indicia is adjacent to the set of second indicia in a first row. The first table of information includes genetic information for at least one of identified clonotypes and cell receptors belonging to a clonotype that are presented in the first visual sequence. The dynamic display of the multi-section data file can provide for analysis of the cellular data from the multi-section data file.

In another aspect, a non-transitory, computer-readable medium storing instructions is provided. The instructions, when executed by a processor, cause the processor to perform operations comprising receiving a data set comprising cellular data, wherein the cellular data includes at least one of an antigen binding specificity value associated with a first cell receptor and a unique molecular identifier (UMI) count associated with the first cell receptor. The operations can further comprise presenting an end user with a visualization tool. The visualization tool can provide a dynamic display of the data set via additional operations that the processor is configured to perform comprising generating a first visual sequence from the data set, displaying, at least a portion of the generated first visual sequence, and displaying, In response to a user interaction with the visualization tool, the antigen binding specificity value or the UMI associated with the cell receptor in conjunction with the at least a portion of the first visual sequence. The first visual sequence includes a set of first indicia corresponding to a first chain of the first cell receptor and a set of second indicia corresponding to a second chain of the first cell receptor, wherein the set of first indicia is adjacent to the set of second indicia in a first row. The dynamic display of the data set can provide for analysis of the cellular data from the data set.

In another aspect, a non-transitory, computer-readable medium storing instructions is provided. The instructions, when executed by a processor, cause the processor to perform operations comprising receiving, a data set comprising cellular data. The operations can further comprise receiving, a selection of a filter, wherein the filter is selected from the group consisting of: UMI Counts/antigen, antigen specificity, barcode, number of barcodes representing a clonotype, CDR3 amino acid sequence, CDR3 bases, gene name, isotype, cluster, amino acid % mutation, nucleotide % mutation, iNKT/MAIT, and combinations thereof. The operations can further comprise presenting an end user with a visualization tool. The visualization tool can provide a dynamic display of the data set via additional operations that the processor is configured to perform comprising generating, a first visual sequence from the data set and, displaying, at least a portion of the generated first visual sequence, generating, a first table of information from the data set and displaying, the first table of information, modifying, the first visual sequence and the first table of information based on the filter, to generate a modified first visual sequence and modified first table of information that is different from the first visual sequence and the first table of information, and displaying, the modified first visual sequence and modified first table of information. The first visual sequence includes a set of first indicia corresponding to a first chain of the first cell receptor and a set of second indicia corresponding to a second chain of the first cell receptor, wherein the set of first indicia is adjacent to the set of second indicia in a first row. The first table of information includes genetic information for at least one of identified clonotypes and cell receptors belonging to a clonotype that are presented in the first visual sequence. The dynamic display of the data set can provide for analysis of the cellular data from the data set.

In another aspect, a computer-readable storage medium encoded with instructions, executable by a processor, for visualizing cellular data, is provided. The instructions can comprise receiving, by the processor, a data set comprising cellular data. The instructions can further comprise receiving, by the processor, a selection of a filter, wherein the filter is selected from the group consisting of UMI Counts/antigen, antigen specificity, barcode, number of barcodes representing a clonotype, CDR amino acid sequence (e.g., CDR3 amino acid sequence), CDR bases (e.g., CDR3 bases), gene name, isotype, cluster, amino acid % mutation, nucleotide % mutation, iNKT/MAIT, and combinations thereof. The instructions can further comprise presenting an end user with a visualization tool. The visualization tool can provide a dynamic display of the data set by generating, by the processor, a first plot from the data set, and displaying the first plot, and modifying, by the processor, the first plot based on the filter, to generate a modified first plot that is different from the first plot, and displaying the modified first plot. The dynamic display of the data set can provide for analysis of the cellular data from the data set.

In another aspect, a computer implemented method for visualizing cellular data is disclosed. The method can comprise receiving, by the processor, a data set comprising cellular data. The method can further comprise receiving, by the processor, a filter, wherein the filter is selected from the group consisting of UMI Counts/antigen, antigen specificity, barcode, number of barcodes representing a clonotype, CDR amino acid sequence (e.g., CDR3 amino acid sequence), CDR bases (e.g., CDR3 bases), gene name, isotype, cluster, amino acid % mutation, nucleotide % mutation, iNKT/MAIT, and combinations thereof. The method can further comprise presenting an end user with a visualization tool. The visualization tool can provide a dynamic display of the data set by generating, by the processor, a first plot from the data set, and displaying the first plot, and modifying, by the processor, the first plot based on the filter, to generate a modified first plot that is different from the first plot, and displaying the modified first plot. The dynamic display of the data set can provide for analysis of the cellular data from the data set.

In another aspect, a system is disclosed. The system can comprise a processor and a memory in communication with the processor. The memory can store instructions for receiving, by the processor, a data set comprising cellular data. The memory can store instructions for receiving, by the processor, a selection of a filter, wherein the filter is selected from the group consisting of UMI Counts/antigen, antigen specificity, barcode, number of barcodes representing a clonotype, CDR amino acid sequence (e.g., CDR3 amino acid sequence), CDR bases (e.g., CDR3 bases), gene name, isotype, cluster, amino acid % mutation, nucleotide % mutation, iNKT/MAIT, and combinations thereof. The memory can store instructions for presenting an end user with a visualization tool. The visualization tool can provide a dynamic display of the data set by generating, by the processor, a first plot from the data set, and displaying the first plot, and modifying, by the processor, the first plot based on the filter, to generate a modified first plot that is different from the first plot, and displaying the modified first plot. The dynamic display of the data set can provide for analysis of the cellular data from the data set.

In an aspect, a computer-readable storage medium encoded with instructions, executable by a processor, for visualizing cellular data, is provided. The instructions can comprise receiving, by the processor, a data set comprising cellular data. The instructions can comprise receiving, by the processor, a selection of a filter, wherein the filter is selected from a plurality of properties of the data set. The instructions can comprise presenting an end user with a visualization tool. The visualization tool can provide a dynamic display of the data set by generating, by the processor, a first plot from the data set, and displaying the first plot, generating, by the processor, a first table of information from the data set, and displaying the table, and modifying, by the processor, the first plot and the first table based on the filter, to generate a modified first plot and modified first table of information that is different from the first plot and first table, and displaying the modified first plot and modified first table. The dynamic display of the data set can provide for analysis of the cellular data from the data set.

In another aspect, a computer implemented method for visualizing cellular data is disclosed. The method can comprise receiving, by the processor, a data set comprising cellular data. The method can comprise receiving, by the processor, a selection of a filter, wherein the filter is selected from a plurality of properties of the data set. The method can comprise presenting an end user with a visualization tool. The visualization tool can provide a dynamic display of the data set by generating, by the processor, a first plot from the data set, and displaying the first plot, generating, by the processor, a first table of information from the data set, and displaying the table, and modifying, by the processor, the first plot and the first table based on the filter, to generate a modified first plot and modified first table of information that is different from the first plot and first table, and displaying the modified first plot and modified first table. The dynamic display of the data set can provide for analysis of the cellular data from the data set.

In another aspect, a system is provided. The system can comprise a processor and a memory in communication with the processor. The memory can store instructions for receiving, by the processor, a data set comprising cellular data. The memory can store instructions for receiving, by the processor, a selection of a filter, wherein the filter is selected from a plurality of properties of the data set. The memory can store instructions for presenting an end user with a visualization tool. The visualization tool can provide a dynamic display of the data set by generating, by the processor, a first plot from the data set, and displaying the first plot, generating, by the processor, a first table of information from the data set, and displaying the table, and modifying, by the processor, the first plot and the first table based on the filter, to generate a modified first plot and modified first table of information that is different from the first plot and first table, and displaying the modified first plot and modified first table. The dynamic display of the data set can provide for analysis of the cellular data from the data set.

These and other aspects and implementations are discussed in detail herein. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification.

### BRIEF DESCRIPTION OF FIGURES

The accompanying drawings are not intended to be drawn to scale. Like reference numbers and designations in the various drawings indicate like elements. For purposes of clarity, not every component may be labeled in every drawing. In the drawings:

FIG. 1 illustrates an interactive visualization system, in accordance with various embodiments.

FIG. 2 illustrates an interactive visualization method, in accordance with various embodiments.

FIG. 3 illustrates a first example visualization, in accordance with various embodiments.

FIG. 4 illustrates a second example visualization, in accordance with various embodiments.

FIG. 5 illustrates a third example visualization, in accordance with various embodiments.

FIG. 6 illustrates an example workflow for the operation of a visualization tool for cellular data, in accordance with various embodiments.

FIG. 7 illustrates an example workflow for the operation of a visualization tool for cellular data, in accordance with various embodiments.

FIG. 8 illustrates an example output display of a visualization tool for cellular data, in accordance with various embodiments.

FIG. 9 illustrates an example filter panel of a visualization tool for cellular data, in accordance with various embodiments.

FIGS. 10A to 10H illustrate example output displays of a visualization tool for cellular data, in accordance with various embodiments.

FIG. 11A illustrates an example output display of a visualization tool for cellular data, in accordance with various embodiments.

FIG. 11B illustrates an example output display of a visualization tool for cellular data, in accordance with various embodiments.

FIG. 12 illustrates a block diagram that illustrates a computer system, in accordance with various embodiments.

FIG. 13 illustrates an interactive visualization method, in accordance with various embodiments.

FIGS. 14A to 14I illustrate example output displays of a visualization tool for cellular data, in accordance with various embodiments.

FIG. 15 illustrates a visual sequence of the example output displays of FIGS. 14A to 14G.

FIG. 16 illustrates an example workflow for the operation of a visualization tool for cellular data, in accordance with various embodiments.

FIG. 17 illustrates an example workflow for the operation of a visualization tool for cellular data, in accordance with various embodiments.

FIG. 18 illustrates an example workflow for antigen specificity analysis, in accordance with various embodiments.

FIGS. 19A-19C illustrate an example output display of a visualization tool for cellular data, in accordance with various embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

The following description of various embodiments is exemplary and explanatory only and is not to be construed as limiting or restrictive in any way. Other embodiments, features, objects, and advantages of the present teachings will be apparent from the description and accompanying drawings, and from the claims.

It should be understood that any use of subheadings herein are for organizational purposes, and should not be read to limit the application of those subheaded features to the various embodiments herein. Each and every feature described herein is applicable and usable in all the various embodiments discussed herein and that all features described herein can be used in any contemplated combination, regardless of the specific example embodiments that are described herein. It should further be noted that exemplary description of specific features are used, largely for informational purposes, and not in any way to limit the design, subfeature, and functionality of the specifically described feature.

Unless defined otherwise, all technical and scientific terms used herein have the same meaning as commonly understood by one of ordinary skill in the art to which their various embodiments belong.

All publications mentioned herein are incorporated herein by reference for the purpose of describing and disclosing devices, compositions, formulations and methodologies which are described in the publication and which might be used in connection with the present disclosure.

As used herein, the terms “comprise”, “comprises”, “comprising”, “contain”, “contains”, “containing”, “have”, “having” “include”, “includes”, and “including” and their variants are not intended to be limiting, are inclusive or open-ended and do not exclude additional, unrecited additives, components, integers, elements or method steps. For example, a process, method, system, composition, kit, or apparatus that comprises a list of features is not necessarily limited only to those features but may include other features not expressly listed or inherent to such process, method, system, composition, kit, or apparatus.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and techniques described herein are those well-known and commonly used in the art.

DNA (deoxyribonucleic acid) is a chain of nucleotides consisting of 4 types of nucleotides; A (adenine), T (thymine), C (cytosine), and G (guanine), and that RNA (ribonucleic acid) is comprised of 4 types of nucleotides; A, U (uracil), G, and C. Certain pairs of nucleotides specifically bind to one another in a complementary fashion (called complementary base pairing). That is, adenine (A) pairs with thymine (T) (in the case of RNA, however, adenine (A) pairs with uracil (U)), and cytosine (C) pairs with guanine (G). When a first nucleic acid strand binds to a second nucleic acid strand made up of nucleotides that are complementary to those in the first strand, the two strands bind to form a double strand. As used herein, “nucleic acid sequencing data,” “nucleic acid sequencing information,” “nucleic acid sequence,” “genomic sequence,” “genetic sequence,” or “fragment sequence,” or “nucleic acid sequencing read” denotes any information or data that is indicative of the order of the nucleotide bases (e.g., adenine, guanine, cytosine, and thymine/uracil) in a molecule (e.g., whole genome, whole transcriptome, exome, oligonucleotide, polynucleotide, fragment, etc.) of DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronical-based systems, etc.

A “polynucleotide”, “nucleic acid”, or “oligonucleotide” refers to a linear polymer of nucleosides (including deoxyribonucleosides, ribonucleosides, or analogs thereof) joined by internucleosidic linkages. Typically, a polynucleotide comprises at least three nucleosides. Usually oligonucleotides range in size from a few monomeric units, e.g. 3-4, to several hundreds of monomeric units. Whenever a polynucleotide such as an oligonucleotide is represented by a sequence of letters, such as “ATGCCTG,” it will be understood that the nucleotides are in 5′→3′ order from left to right and that “A” denotes deoxyadenosine, “C” denotes deoxycytidine, “G” denotes deoxyguanosine, and “T” denotes thymidine, unless otherwise noted. The letters A, C, G, and T may be used to refer to the bases themselves, to nucleosides, or to nucleotides comprising the bases, as is standard in the art.

The phrase “next generation sequencing” (NGS) refers to sequencing technologies having increased throughput as compared to traditional Sanger- and capillary electrophoresis-based approaches, for example with the ability to generate hundreds of thousands of relatively small sequence reads at a time. Some examples of next generation sequencing techniques include, but are not limited to, sequencing by synthesis, sequencing by ligation, and sequencing by hybridization. More specifically, the MISEQ, HISEQ, NEXTSEQ, and NOVASEQ Systems of Illumina, the DNBSEQ and BGISEQ platforms of Beijing Genomics Institute (BGI), the GRIDION and PROMETHION Systems of Oxford Nanopore Technologies, PACBIO SEQUEL Systems of Pacific Biosciences, and the Personal Genome Machine (PGM) and SOLiD Sequencing System of Life Technologies Corp, provide massively parallel sequencing of whole or targeted genomes. The SOLID System and associated workflows, protocols, chemistrics, etc. are described in more detail in PCT Publication No. WO 2006/084132, entitled “Reagents, Methods, and Libraries for Bead-Based Sequencing,” international filing date Feb. 1, 2006, U.S. patent application Ser. No. 12/873,190, entitled “Low-Volume Sequencing System and Method of Use,” filed on Aug. 31, 2010, and U.S. patent application Ser. No. 12/873,132, entitled “Fast-Indexing Filter Wheel and Method of Use,” filed on Aug. 31, 2010, the entirety of each of these applications being incorporated herein by reference thereto.

The phrase “sequencing run” refers to any step or portion of a sequencing experiment performed to determine some information relating to at least one biomolecule (e.g., nucleic acid molecule).

As used herein, the phrase “genomic features” can refer to a genome region with some annotated function (e.g., a gene, protein coding sequence, mRNA, tRNA, rRNA, repeat sequence, inverted repeat, miRNA, siRNA, etc.) or a genetic/genomic variant (e.g., single nucleotide polymorphism/variant, insertion/deletion sequence, copy number variation, inversion, etc.), which denotes a single or a grouping of genes (in DNA or RNA) that have undergone changes as referenced against a particular species or sub-populations within a particular species due to mutations, recombination/crossover or genetic drift.

In general, the methods and systems described herein accomplish sequencing of nucleic acid molecules including, but not limited to, DNA (e.g., genomic DNA), RNA (e.g., mRNA, including full-length mRNA transcripts, and small RNAs, such as miRNA, tRNA, and rRNA), and cDNA. In various embodiments, the methods and systems described herein accomplish nucleic acid sequencing of nucleic acid molecules (e.g., DNA, RNA, and mRNA). In various embodiments, the methods and systems described herein accomplish nucleic acid sequencing of immune cell receptor sequences (e.g., DNA, RNA, and mRNA). In various embodiments, the methods and systems described herein can accomplish transcriptome sequencing, e.g., whole transcriptome sequencing of mRNA encoding immune cell receptors. In some embodiments, the methods and systems described herein can also accomplish targeted nucleic acid sequencing of nucleic acid molecules (e.g., DNA, RNA, and mRNA). In various embodiments, the methods and systems described herein accomplish single cell nucleic acid sequencing, for example, single cell nucleic acid sequencing of nucleic acid molecules (e.g., RNA and mRNA) encoding immune cell receptors of single cells, such as B cell receptors (BCRs) and T cell receptors (TCRs).

In various embodiments, the methods and systems described herein can include high-throughput sequencing technologies, e.g., high-throughput DNA and RNA sequencing technologies. In various embodiments, the methods and systems described herein can include high-throughput, higher accuracy short-read DNA and RNA sequencing technologies. In various embodiments, the methods and systems described herein can include long-read RNA sequencing, e.g., by sequencing cDNA transcripts in their entirety without assembly. In various embodiments, the methods and systems described herein can also, for example, segment long nucleic acid molecules into smaller fragments that can be sequenced using high-throughput, higher accuracy short-read sequencing technologies, and that segmentation is accomplished in a manner that allows the sequence information derived from the smaller fragments to retain the original long range molecular sequence context, i.e., allowing the attribution of shorter sequence reads to originating longer individual nucleic acid molecules. By attributing sequence reads to an originating longer nucleic acid molecule, one can gain significant characterization information for that longer nucleic acid sequence that one cannot generally obtain from short sequence reads alone. This long-range molecular context is not only preserved through a sequencing process, but is also preserved through the targeted enrichment process used in targeted sequencing approaches.

In general, the methods and systems described herein are directed to single cell analysis (including single- and multi-modal analyses) of nucleic acid sequencing of nucleic acids (e.g., RNA and mRNA) encoding immune cell receptors of single cells, such as B cell receptors (BCRs) and T cell receptors (TCRs). Single cell analysis, including single cell multi-modal analyses (e.g., single cell immune cell receptor sequencing combined with, for example, gene expression, protein expression, and/or antigen capture technologies), as well as processing and sequencing of nucleic acids, in accordance with the methods and systems described in the present application are described in further detail, for example, in U.S. Pat. Nos. 9,689,024; 9,701,998; 10,011,872; 10,221,442; 10,337,061; 10,550,429; 10,273,541; and U.S. Pat. Pub. 20180105808, which are all herein incorporated by reference in their entirety for all purposes and in particular for all written description, figures and working examples directed to processing nucleic acids and sequencing and other characterizations of genomic material.

The term “barcode,” as used herein, generally refers to a label, or identifier, that conveys or is capable of conveying information about an analyte. A barcode can be part of an analyte. A barcode can be independent of an analyte. A barcode can be a tag attached to an analyte (e.g., nucleic acid molecule) or a combination of the tag in addition to an endogenous characteristic of the analyte (e.g., size of the analyte or end sequence(s)). A barcode may be unique. Barcodes can have a variety of different formats. For example, barcodes can include: polynucleotide barcodes; random nucleic acid and/or amino acid sequences; and synthetic nucleic acid and/or amino acid sequences. A barcode can be attached to an analyte in a reversible or irreversible manner. A barcode can be added to, for example, a fragment of a deoxyribonucleic acid (DNA) or ribonucleic acid (RNA) sample before, during, and/or after sequencing of the sample. Barcodes can allow for identification and/or quantification of individual sequencing-reads.

The terms “adaptor(s)”, “adapter(s)” and “tag(s)” may be used synonymously. An adaptor or tag can be coupled to a polynucleotide sequence to be “tagged” by any approach, including ligation, hybridization, or other approaches.

The term “sequencing,” as used herein, generally refers to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA). Sequencing can be performed by various systems currently available, such as, without limitation, a sequencing system by Illumina®, Pacific Biosciences (PacBio®), Oxford Nanopore®, or Life Technologies (Ion Torrent®). Alternatively or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

The term “bead,” as used herein, generally refers to a particle. The bead may be a solid or semi-solid particle. The bead may be a gel bead. The gel bead may include a polymer matrix (e.g., matrix formed by polymerization or cross-linking). The polymer matrix may include one or more polymers (e.g., polymers having different functional groups or repeat units). Polymers in the polymer matrix may be randomly arranged, such as in random copolymers, and/or have ordered structures, such as in block copolymers. Cross-linking can be via covalent, ionic, or inductive, interactions, or physical entanglement. The bead may be a macromolecule. The bead may be formed of nucleic acid molecules bound together. The bead may be formed via covalent or non-covalent assembly of molecules (e.g., macromolecules), such as monomers or polymers. Such polymers or monomers may be natural or synthetic. Such polymers or monomers may be or include, for example, nucleic acid molecules (e.g., DNA or RNA). The bead may be formed of a polymeric material. The bead may be magnetic or non-magnetic. The bead may be rigid. The bead may be flexible and/or compressible. The bead may be disruptable or dissolvable. The bead may be a solid particle (e.g., a metal-based particle including but not limited to iron oxide, gold or silver) covered with a coating comprising one or more polymers. Such coating may be disruptable or dissolvable.

As used herein, the terms “barcoded nucleic acid molecule” and “barcoded polynucleotide” are used interchangeably herein to generally refer to a nucleic acid molecule that results from, for example, the processing of a nucleic acid barcode molecule with a nucleic acid sequence (e.g., nucleic acid sequence complementary to a nucleic acid primer sequence encompassed by the nucleic acid barcode molecule). The nucleic acid sequence may be a targeted sequence or a non-targeted sequence. The nucleic acid barcode molecule may be coupled to or attached to the nucleic acid molecule comprising the nucleic acid sequence. For example, a nucleic acid barcode molecule described herein may be hybridized to an analyte (e.g., a messenger RNA (mRNA) molecule) of a cell. Reverse transcription can generate a barcoded nucleic acid molecule that has a sequence corresponding to the nucleic acid sequence of the mRNA and the barcode sequence (or a reverse complement thereof). The processing of the nucleic acid molecule comprising the nucleic acid sequence, the nucleic acid barcode molecule, or both, can include a nucleic acid reaction, such as, in non-limiting examples, reverse transcription, nucleic acid extension, ligation, etc. The nucleic acid reaction may be performed prior to, during, or following barcoding of the nucleic acid sequence to generate the barcoded nucleic acid molecule. For example, the nucleic acid molecule comprising the nucleic acid sequence may be subjected to reverse transcription and then be attached to the nucleic acid barcode molecule to generate the barcoded nucleic acid molecule, or the nucleic acid molecule comprising the nucleic acid sequence may be attached to the nucleic acid barcode molecule and subjected to a nucleic acid reaction (e.g., extension, ligation) to generate the barcoded nucleic acid molecule. A barcoded nucleic acid molecule may serve as a template, such as a template polynucleotide, that can be further processed (e.g., amplified) and sequenced to obtain the target nucleic acid sequence. For example, in the methods and systems described herein, a barcoded nucleic acid molecule may be further processed (e.g., amplified) and sequenced to obtain the nucleic acid sequence of the nucleic acid molecule (e.g., mRNA).

The term “sample,” as used herein, generally refers to a biological sample of a subject. The biological sample may comprise any number of macromolecules, for example, cellular macromolecules. The sample may be a cell sample. The sample may be a cell line or cell culture sample. The sample can include one or more cells. The sample can include one or more microbes. The biological sample may be a nucleic acid sample or protein sample. The biological sample may also be a carbohydrate sample or a lipid sample. The biological sample may be derived from another sample. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a cheek swab. The sample may be a plasma or serum sample. The sample may be a cell-free or cell free sample. A cell-free sample may include extracellular polynucleotides. Extracellular polynucleotides may be isolated from a bodily sample that may be selected from the group consisting of blood, plasma, serum, urine, saliva, mucosal excretions, sputum, stool and tears.

The term “biological particle” may be used herein to generally refer to a discrete biological system derived from a biological sample. The biological particle may be a macromolecule. The biological particle may be a small molecule. The biological particle may be a virus. The biological particle may be a cell or derivative of a cell. The biological particle may be an organelle. The biological particle may be a nucleus of a cell. The biological particle may be a rare cell from a population of cells. The biological particle may be any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological particle may be a constituent of a cell. The biological particle may be or may include DNA, RNA, organelles, proteins, or any combination thereof. The biological particle may be or may include a matrix (e.g., a gel or polymer matrix) comprising a cell or one or more constituents from a cell (e.g., cell bead), such as DNA, RNA, organelles, proteins, or any combination thereof, from the cell. The biological particle may be obtained from a tissue of a subject. The biological particle may be a hardened cell. Such hardened cell may or may not include a cell wall or cell membrane. The biological particle may include one or more constituents of a cell, but may not include other constituents of the cell. An example of such constituents is a nucleus or an organelle. A cell may be a live cell. The live cell may be capable of being cultured, for example, being cultured when enclosed in a gel or polymer matrix, or cultured when comprising a gel or polymer matrix.

The term “macromolecular constituent,” as used herein, generally refers to a macromolecule contained within or from an biological particle. The macromolecular constituent may comprise a nucleic acid. In some cases, the biological particle may be a macromolecule. The macromolecular constituent may comprise DNA. The macromolecular constituent may comprise RNA. The RNA may be coding or non-coding. The RNA may be messenger RNA (mRNA), ribosomal RNA (rRNA) or transfer RNA (tRNA), for example. The RNA may be a transcript. The RNA may be small RNA that are less than 200 nucleic acid bases in length, or large RNA that are greater than 200 nucleic acid bases in length. Small RNAs may include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA) and small rDNA-derived RNA (srRNA). The RNA may be double-stranded RNA or single-stranded RNA. The RNA may be circular RNA. The macromolecular constituent may comprise a protein. The macromolecular constituent may comprise a peptide. The macromolecular constituent may comprise a polypeptide.

The term “molecular tag,” as used herein, generally refers to a molecule capable of binding to a macromolecular constituent. The molecular tag may bind to the macromolecular constituent with high affinity. The molecular tag may bind to the macromolecular constituent with high specificity. The molecular tag may comprise a nucleotide sequence. The molecular tag may comprise a nucleic acid sequence. The nucleic acid sequence may be at least a portion or an entirety of the molecular tag. The molecular tag may be a nucleic acid molecule or may be part of a nucleic acid molecule. The molecular tag may be an oligonucleotide or a polypeptide. The molecular tag may comprise a DNA aptamer. The molecular tag may be or comprise a primer. The molecular tag may be, or comprise, a protein. The molecular tag may comprise a polypeptide. The molecular tag may be a barcode.

The term “B cells”, also known as B lymphocytes, refer to a type of white blood cell of the small lymphocyte subtype. They function in the humoral immunity component of the adaptive immune system by expressing and/or secreting antibodies. Additionally, B cells present antigens (they are also classified as professional antigen-presenting cells (APCs)) and secrete cytokines. In mammals, B cells mature in the bone marrow, which is at the core of most bones. In birds, B cells mature in the bursa of Fabricius, an immune organ where they were first discovered by Chang and Glick, (B for bursa) and not from bone marrow as commonly believed. B cells, unlike the other two classes of lymphocytes, T cells and natural killer cells, express B cell receptors (BCRs) on their cell membrane or secrete their BCRs if they have differentiated into long-lived plasma cells. BCRs allow a B cell to bind to specific antigens, against which it will initiate an antibody response.

The term “T cell”, also known as T lymphocytes, refer to a type of an adaptive immune cell. T cells develops in the thymus gland, hence the name T cell, and play a central role in the immune response of the body. T cells can be distinguished from other lymphocytes by the presence of a T cell receptor (TCR) on the cell surface. These immune cells originate as precursor cells, derived from bone marrow, and then develop into several distinct types of T cells once they have migrated to the thymus gland. T cell differentiation continues even after they have left the thymus. T cells include, but are not limited to, helper T cells, cytotoxic T cells, memory T cells, regulatory T cells, and killer T cells. Helper T cells stimulate B cells to make antibodies and help killer cells develop. Based on the T cell receptor chain, T cells can also include T cells that express αβ TCR chains, T cells that express γδ TCR chains, as well as unique TCR co-expressors (i.e., hybrid αβ-γδ T cells) that co-express the αβ and γδ TCR chains.

T cells can also include engineered T cells that can attack specific cancer cells. A patient's T cells can be collected and genetically engineered to produce chimeric antigen receptors (CAR). These engineered T cells are called CAR T cells, which forms the basis of the developing technology called CAR-T therapy. These engineered CAR T cells are grown by the billions in the laboratory and then infused into a patient's body, where the cells are designed to multiply and recognize the cancer cells that express the specific protein. This technology, also called adoptive cell transfer is emerging as a potential next-generation immunotherapy treatment.

T cells, such as the killer T cells can directly kill cells that have already been infected by a foreign invader. T cells can also use cytokines as messenger molecules to send chemical instructions to the rest of the immune system to ramp up its response. Activating T cells against cancer cells is the basis behind checkpoint inhibitors, a relatively new class of immunotherapy drugs that have recently been approved to treat lung cancer, melanoma, and other difficult cancers. Cancer cells often evade patrolling T cells by sending signals that make them seem harmless. Checkpoint inhibitors disrupt those signals and prompt the T cells to attack the cancer cells.

The term “naïve”, as used herein, can refer to B-lymphocytes or T-lymphocytes that have not yet reacted with an epitope of an antigen or that have a cellular phenotype consistent with that of a lymphocyte that has not yet responded to antigen-specific activation after clonal licensing.

The term “Fab”, also referred to as an antigen-binding fragment, refers to the variable portions of an antibody molecule with a paratope that enables the binding of a given epitope of a cognate antigen. The amino acid and nucleotide sequences of the Fab portion of antibody molecules are hypervariable. This is in contrast to the “Fc” or crystallizable fragment, which is relatively constant and encodes the isotype for a given antibody; this region can also confer additional functional capacity through processes such as antibody-dependent complement deposition, cellular cytotoxicity, cellular trogocytosis, and cellular phagocytosis.

The phrase “clonal selection” refers to the selection and activation of specific B lymphocytes and T lymphocytes by the binding of epitopes to B cell receptors or T cell receptors with a corresponding fit and the subsequent elimination (negative selection) or licensing for clonal expansion (positive selection) of a B or T lymphocyte after binding of an antigenic determinant.

The phrase “clonal expansion” refers to the proliferation of B lymphocytes and T lymphocytes activated by clonal selection in order to produce a clonal population of daughter cells with the same antigen specificity and functional capacity. In the case of T lymphocytes this antigen specificity is exact at the nucleotide and protein level and in the case of B lymphocytes this antigen specificity can be exact at the nucleotide and protein level or mutated relative to the parent population by mutations at the nucleotide level (and by extension the protein level). This enables the body to have sufficient numbers of antigen-specific lymphocytes to mount an effective immune response.

The term “cytokines” refers to a wide variety of intercellular regulatory proteins produced by many different cells in the body, which ultimately control every aspect of body defense. Cytokines activate and deactivate phagocytes and immune defense cells, enhance or inhibit the functions of the different immune defense cells, and promote or inhibit a variety of nonspecific body defenses.

The phrase “T helper lymphocytes”, also referred to as helper cells, refer to a type of white blood cell that orchestrate the immune response and enhance the activities of the killer T-cells (those that destroy pathogens) and B cells (antibody and immunoglobulin producers).

The phrase “affinity maturation” refers to the gradual modification of the paratope and entire B cell receptor as a result of somatic hypermutation. B lymphocytes with higher affinity B cell receptors that can 1) bind the epitope more tightly and 2) therefore bind the epitope for a longer period of time are able to proliferate more and survive longer. These B cells can eventually differentiate into plasma cells, which secrete their antibodies and form the basis of serum-mediated immunity.

The phrase “somatic hypermutation” (SHM) refers to a cellular mechanism by which the adaptive immune system adapts to foreign elements confronting it (e.g. viruses, bacteria, biomolecules). A major component of the process of affinity maturation, SHM diversifies B cell receptors used to recognize foreign elements (antigens) and allows the immune system to adapt its response to new threats during the lifetime of an organism. Somatic hypermutation involves a programmed process of mutation predominantly affecting select framework and complementarity-determining regions of immunoglobulin genes. Unlike germline mutation, SHM operates at the level of an organism's individual immune cells. These mutations are not transmitted to the organism's offspring, but are transmitted to daughter cells of individual B cell clones. Mistargeted somatic hypermutation is a likely mechanism in the development of B cell lymphomas and many other cancers. Somatic hypermutation can also lead to the acquisition of non-VDJ template DNA within B cell receptor sequences, such as LAIR1 insertions in malaria-specific neutralizing antibodies.

Somatic hypermutation is a distinct diversification mechanism from isotype switching (also called class switching). Mutations acquired during somatic hypermutation eventually lead to isotype switching, in which a B cell's antibody can be coupled to different functions by switching to a different Fc/constant region sequence. Isotype switching is an irreversible process, in that once a B cell has switched from a given constant region (e.g. IGHM) to a new constant region (e.g. IGHA1) it can no longer use the IgM constant region as the DNA encoding the IgM Fe is excised and removed during isotype switching.

The term “contig”, originating from the term “contiguous”, refers to a set of overlapping DNA segments that together represent a consensus region of DNA. In bottom-up sequencing projects, a contig refers to overlapping sequence data (reads); in top-down sequencing projects, contig refers to the overlapping clones that form a physical map of the genome that is used to guide sequencing and assembly. Contigs can thus refer both to overlapping DNA sequences and to overlapping physical segments (fragments) contained in clones depending on the context. Note that clone, in reference to overlapping clones, refers to individual bacteria or constructs (e.g. phagemids, cosmids, etc.) containing distinct insertions of genomes that were utilized in early efforts to map genomes.

The phrase “heavy chain” refers to the large polypeptide subunit of an antibody (immunoglobulin). The first recombination event to occur is between one D and one J gene segment of the heavy chain locus. Any DNA between these two gene segments is deleted. This D-J recombination is followed by the joining of one V gene segment, from a region upstream of the newly formed DJ complex, forming a rearranged VDJ gene segment. All other gene segments between V and D segments are now deleted from the cell's genome. Primary transcript (unspliced RNA) is generated containing the VDJ region of the heavy chain and both the constant mu and delta chains (Cμ and Cδ) (i.e., the primary transcript contains the segments: V-D-J-Cμ-Cδ). The primary RNA is processed to add a polyadenylated (poly-A) tail after the Cu chain and to remove sequence between the VDJ segment and this constant gene segment. Translation of this mRNA leads to the production of the IgM heavy chain protein and the IgD heavy chain protein (its splice variant). Expression of the immunoglobulin heavy chain with one or more surrogate light chains constitutes the pre-B cell receptor that allows a B cell to undergo selection and maturation.

The phrase “light chain” refers to the small polypeptide subunit of an antibody (immunoglobulin). The kappa (κ) and lambda (λ) chains of the immunoglobulin light chain loci rearrange in a very similar way, except that the light chains lack a D segment. In other words, the first step of recombination for the light chains involves the joining of the V and J chains to give a VJ complex before the addition of the constant chain gene during primary transcription. Translation of the spliced mRNA for either the kappa or lambda chains results in formation of the Ig κ or Ig λ light chain protein. Assembly of the Ig μ heavy chain and one of the light chains results in the formation of membrane bound form of the immunoglobulin IgM that is expressed on the surface of the immature B cell. B cells may express up to two heavy chains and/or two light chains in respectively rare and uncommon instances through a phenomenon known as allelic inclusion. This phenomenon can only be directly observed using single-cell technologies, though it can be inferred with a degree of uncertainty using a combination of bulk sequencing technologies and probabilistic inference via an extension of the birthday paradox.

The phrase “complementarity-determining regions” (CDRs) refers to part of the variable chains in immunoglobulins (antibodies) and T cell receptors, generated by B cells and T cells respectively, where these molecules are particularly hypervariable. The antigen-binding site of most antibodies and T cell receptors is typically distributed across these CDRs, collectively forming a paratope. However, there are many documented examples of paratopes that enable antigen recognition that fall outside of the CDRs. As the most variable parts of the molecules, CDRs are crucial to the diversity of antigen specificities and immune cell receptor sequences generated by lymphocytes.

V(D)J recombination is a genetic recombination mechanism that occurs in developing lymphocytes during the early stages of T and B cell maturation. Through somatic recombination, this mechanism produces a highly diverse repertoire of antibodies/immunoglobulins and T cell receptors (TCRs) found in B cells and T cells, respectively. This process is a defining feature of the adaptive immune system and these receptors are defining features of adaptive immune cells.

V(D)J recombination occurs in the primary immune organs (bone marrow for B cells and thymus for T cells) and in a generally random fashion. The process leads to the rearranging of variable (V), joining (J), and in some cases, diversity (D) gene segments. As discussed above, the heavy chain possesses numerous V, D, and J gene segments, while the light chain possesses only V and J gene segments. The process ultimately results in novel amino acid sequences in the antigen-binding regions of immunoglobulins and TCRs that allow for the recognition of antigens from nearly all pathogens including, for example, bacteria, viruses, and parasites. Furthermore, the recognition can also be allergic in nature or may recognize host tissues and lead to autoimmunity.

Human antibody molecules, including B cell receptors (BCRs), include both heavy and light chains, each of which contains both constant (C) and variable (V) regions, and are genetically encoded on three loci. The first is the immunoglobulin heavy locus on chromosome 14, containing the gene segments for the immunoglobulin heavy chain. The second is the immunoglobulin kappa (κ) locus on chromosome 2, containing the gene segments for part of the immunoglobulin light chain. The third is the immunoglobulin lambda (λ) locus on chromosome 22, containing the gene segments for the remainder of the immunoglobulin light chain.

Each heavy or light chain contains multiple copies of different types of gene segments for the variable regions of the antibody proteins. For example, the human immunoglobulin heavy chain region contains two C gene segments (Cμ and Cδ), 44 V gene segments, 27 D gene segments and 6 J gene segments. The number of given segments present in any individual can vary, as these gene segments are carried in haplotypes; for this reason, inference of both the alleles present within an individuals and the germline sequence of those alleles is an important step in correctly identifying B cell clonotypes. The light chains possess two C gene segments (Cλ and Cκ) and numerous V and J gene segments, but do not have D gene segments. DNA rearrangement causes one copy of each type of gene segment to mate with any given lymphocyte, generating a substantial antibody repertoire. Approximately 10¹⁴ combinations are possible, with 1.5×10² to 3×10³ potentially removed via self-reactivity.

Accordingly, each naïve B cell makes an antibody with a unique Fab site through a series of gene recombinations, and later mutations, with the specific molecules of the given antibody attaching to the B cell's surface as a B cell receptor (BCR). These BCRs are then available to react with epitopes of an antigen.

When the immune system encounters an antigen, epitopes of that antigen will be presented to many B lymphocytes. B lymphocytes may first rearrange a heavy chain that enables pre-B cell receptor ligand binding. B lymphocytes that bind multivalent self-targets after rearrangement of the light chain too strongly are eliminated and die or undergo a secondary recombination event, while B cells that do not bind self-targets too strongly are licensed to exit the bone marrow. The latter becomes available to respond to non-self antigens and to undergo clonal expansion. This process is known as clonal selection.

Cytokines produced by activated CD4 T helper lymphocytes enable those activated B lymphocytes (B cells) to rapidly proliferate to produce large clones of thousands of identical B cells. More specifically, when under threat (i.e., via bacteria, virus, etc.), the body releases white blood cells by the immune system. CD4 T lymphocytes help the response to a threat by triggering the maturation of other types of white blood cell. They produce special proteins, called cytokines, have plural functions, including the ability to summon all of the other immune cells to the area, and also the ability to cause nearby cells to differentiate (become specialized) into mature B cells and T cells.

Accordingly, while only a few B cells in the body may have an antibody molecule that can bind a particular epitope, eventually many thousands of cells are produced with the right specificity, allowing the body's immune system to act en masse. This is referred to as clonal expansion. Natural phenomena such as IgA deficiency and murine transgenic models have shown that there are multiple paths by which a B cell receptor can acquire novel antigen specificity even from a very limited repertoire through the processes of somatic hypermutation and affinity maturation.

As the B cells proliferate, they undergo affinity maturation as a result of somatic hypermutation. This allows the B cells to “fine-tune” the paratopes of the antibody to more effectively fit with the recognized epitopes. B cells with high affinity B cell receptors on their surface bind epitopes more tightly and for a longer period of time, which enables these cells to selectively proliferate. Over the course of this proliferation and expansion, these variant B cells differentiate into plasma cells that synthesize and secrete vast quantities of antibodies with Fab sites that fit the target epitopes very precisely.

The phrase “immune cell” refers to a cell that is part of the immune system and that helps the body fight infections and other diseases. Immune cells include innate immune cells (such as basophils, dendritic cells, neutrophils, etc.) that are the first line of the body's defense and are deployed to help attack the invading foreign cells (e.g., cancer cells) and pathogens. The innate immune cells can quickly respond to foreign cells and pathogens to fight infection, battle a virus, or defend the body against bacteria. Immune cells can also include adaptive immune cells (such as lymphocytes including B cells and T cells). The adaptive immune cells can come into action when an invading foreign cells or pathogens slip through the first line of body's defense mechanism. The adaptive immune cells can take longer to develop, because their behaviors evolve from learned experiences, but they can tend to live longer than innate immune cells. Adaptive immune cells remember foreign invaders after their first encounter and fight them off the next time they enter the body. Both types of immune cells employ important natural defenses in helping the body fight foreign cells and pathogens for fighting infections and other diseases.

Accordingly, the immune cells of the disclosure can include, but are not limited to, neutrophils, eosinophils, basophils, mast cells, monocytes, macrophages, dendritic cells, natural killer cells, and lymphocytes (such as B cells and T cells). The immune cells of the disclosure can further include dual expresser cells or DE (such as unique dual-receptor-expressing lymphocytes that co-express functional B cell receptor (BCR) and T cell receptor (TCR)), cells with adaptive immune receptors that may diversify or may not diversify (including immune cells expressing a chimeric antigen receptor with a fixed nucleotide sequence or with the capacity to mutate), and TCR co-expressors (i.e., hybrid αβ-γδ T cells) that co-express both αβ and γδ TCR chains.

The phrase “immune cell receptor”, “immune receptor”, or “immunologic receptor” refers to a receptor or immune cell receptor sequence, usually on a cell membrane, which can recognize components of pathogenic microorganisms (e.g., components of bacterial cell wall, bacterial flagella or viral nucleic acids) and foreign cells (e.g., cancer cells), which are foreign and not found naturally on the host cells, or binds to a target molecule (for example, a cytokine), and causes a response in the immune system. The immune cell receptors of the immune system can include, but are not limited to, pattern recognition receptors (PRRs), Toll-like receptors (TLRs), killer activated and killer inhibitor receptors (KARs and KIRs), complement receptors, Fc receptors, B cell receptors, and T cell receptors.

The phrase “immune cell receptor sequences” of an immune cell receptor include both heavy and light chains, each of which contains both constant (C) and variable (V) regions. For example, B cell receptors (BCRs) or B cell receptor sequences (including human antibody molecules) comprise of immunoglobulin heavy and light chains, each of which contains both constant (C) and variable (V) regions. Each heavy or light chain not only contains multiple copies of different types of gene segments for the variable regions of the antibody proteins, but also contains constant regions. For example, the BCR or human immunoglobulin heavy chain contains two (2) constant (Constant mu (Cμ) and delta (Cδ)) gene segments and forty four (44) Variable (V) gene segments, plus twenty seven (27) Diversity (D) gene segments, and six (6) Joining (J) gene segments. The BCR light chains also possess two (2) constant gene segments ((Constant lambda (Cλ) and kappa (Cκ)) and numerous V and J gene segments, but do not have any D gene segments. DNA rearrangement (i.e., recombination events) in developing B cells can cause one copy of each type of gene segment to go in any given lymphocyte, generating an enormous antibody repertoire. Accordingly, the primary transcript (unspliced RNA) of a BCR heavy chain can be generated containing the VDJ region of the heavy chain and both the constant mu and delta chains (Cμ and Cδ), i.e., the heavy chain primary transcript can contains the segments: V-D-J-Cμ-Cδ). In case of the B cell receptor and human immunoglobulin light chain, the first step of recombination for the light chains involves the joining of the V and J chains to give a VJ complex before the addition of the constant chain gene during primary transcription. Translation of the spliced mRNA for either the constant κ (Cκ) or λ (Cλ) chains results in formation of the Ig κ or Igλ light chain protein.

In general, most T cell receptors (TCR) are composed of an alpha (a) chain and a beta (β) chain, each of which contains both constant (C) and variable (V) regions. Thus, the most common type of a T cell receptor is called an alpha-beta TCR because it is composed of two different chains, one α-chain and one beta β-chain. A less common type of TCR is the gamma-delta TCR, which contains a different set of chains, one gamma (γ) chain and one delta (δ) chain. The T cell receptor genes are similar to immunoglobulin genes for the BCR and undergo similar DNA rearrangement (i.e., recombination events) in developing T cells as for the B cells. For example, the alpha-beta TCR genes also contain multiple V, D, and J gene segments in their beta chains and V and J gene segments in their alpha chains, which are rearranged during the development of the T cells to provide a cell with a unique T cell antigen receptor. Thus, the β-chain of the TCR can contain Vβ-Dβ-Jβ gene segments and constant domain (CB) genes resulting in a Vβ-Dβ-Jβ-Cβ sequence of the TCR β-chain. The rearrangement of the alpha (α) chain of the TCR follows β chain rearrangement, and can include Vα-Jα gene segments and constant domain (Cα) genes resulting in a Vα-Jα-Cα sequence of the TCR α-chain. Similar to the alpha-beta TCRs, the TCR-γ chain is produced by V-J recombinations and can contain Vγ-Jγ gene segments and constant domain (Cγ) genes resulting in a Vγ-Jγ-Cγ sequence of the TCR γ-chain, while the TCR-δ chain is produced using V-D-J recombinations, and can contain Vδ-Dδ-Jδ gene segments and constant domain (Cδ) genes resulting in a Vδ-Dδ-Jδ-Cδ sequence of the TCR δ-chain.

The phrase “immune cell receptor constant region sequence” or “immune receptor constant region sequence” refers to the constant region or constant region sequence of an immune cell receptor. For example, the immune cell receptor constant region sequence or immune receptor constant region sequence can include, but is not limited to, the constant mu (Cμ) and delta (Cδ) region genes and sequences of a BCR and immunoglobulin heavy chain, the constant lambda (Cλ) and kappa (Cκ) region genes and sequences of a BCR and immunoglobulin light chain, the alpha constant (Cα) region genes and sequences of a TCR α-chain sequence, the beta constant (CB) region genes and sequences of a TCR β-chain sequence, the gamma constant (Cγ) region genes and sequences of a TCR γ-chain sequence, and the delta constant (Cδ) region genes and sequences of a TCR δ-chain sequence.

With this understanding of the immune cell's purpose in fighting off attacking foreign antigens, the pharmaceutical industry has strongly focused on designing vaccines with the ability to expand antibody lineages directed towards specific B cells with shared antigen specificity. The pharmaceutical industry has also directed its efforts to identify TCRs, antibodies, and antibody lineages against targets for the purpose of developing large molecule therapeutics for a broad array of disease states including autoimmune disease (anti-inflammatory targets), cancer (checkpoint inhibitors and other targets), and other conditions such as osteoporosis. Similarly, knowing the fine specificities of different antibody lineages elicited by a vaccine is essential to understanding serum neutralization profiles and global epitope maps of an entire virus. This same concept applies to understanding how a patient's adaptive immune system can render drugs such as adalimumab ineffective through the emergence of anti-drug antibodies and distinct anti-drug antibody lineage. Accordingly, it is advantageous to be able to accurately identify cell members of a clonotype, which potentially share common or similar BCRs (or TCRs) or antigen specificity.

To understand what constitutes members of a B cell clonotype, one can start with the original progenitor cell for a given lineage of B cells, this progenitor cell commonly referred to as the parent clone, which is a single cell to which all daughter cells will be genetically related, though their B cell receptors and exact antigen specificity may differ and diverge over time. Collectively, this parent clone and all its daughter cells constitute a clonotype.

As stated above, accurate identification of the members of a clonotype is critical not just from a biological perspective, but also from the biomedical perspective, as correct identification of all of the members of a given clonotype can be useful in the design of vaccines (e.g., which antibody lineages can be expanded by a vaccine or are expanded successfully or unsuccessfully by a vaccine), in the monitoring of B cell-mediated immune disease (e.g., myasthenia gravis, lupus, B cell lymphoma), and in other settings (what antibodies are found in the tumor microenvironment or other immune niches during clinical disease). Known approaches that attempt to group immune cell receptor sequences into groups with shared antigen specificity or members of the same clonotype include, but are not limited to: immcantation, Clonify, GLIPH, TCRdist, VDJTools, MiXCR, AbSolve, and the algorithms described in PMID: 23536288, PMID: 23898164, PMID: 25345460, etc. While some of these algorithms can successfully identify groups of T cells with shared antigen specificity using single-cell data (TCRdist, GLIPH), and the other algorithms use solely bulk receptor sequencing data (i.e., without access to heavy and light chain sequences), none of these algorithms attempt to approximate the true clonotypes for B cells while also attempting to mitigate for sources of noise in the data nor while using the additional specificity found in the antibody light chain (same can apply to T cells as well). Antibody discovery efforts have shown that false-positive antibody candidates are more frequently found in randomly paired antibody libraries than in natively paired antibody libraries, demonstrating the importance of correct clonotype identification from both biological and pharmaceutical perspectives. Further, none of these approaches provide easy visualization and data interaction routines to display a large amount of information about the single cells within a clonotype in a compact and readily interpretable display.

Therefore, in accordance with various embodiments, various systems and methods are provided that display large amounts of information related to clonotype and subclonotype groupings for B cells or T cells in a dynamic and interactive manner.

### Clonotype Data Visualization

When exploring thousands of clonotypes and exact subclonotypes, the cells and VDJ receptor sequences that will interest a user will depend on the user's experiment and scientific question. The user needs to use their knowledge of the experiment and biological system to identify specific clonotypes and exact subclonotypes, which the user will prioritize for further investigation. For this, the user needs to be able to process the multi-dimensional information that each clonotype is associated with, such as VDJ gene, CDR sequence, antigen specificity score, clonotype size, and gene expression-based clustering. The provided systems and methods enable a user to explore, visualize, and filter a massive amount of cellular data, thereby eliminating the step of processing thousands of multi-dimensional data points, which would be infeasible for humans to do by hand in a way that the information gleaned from the data still has utility by the time the processing by hand is completed. For instance, the provided systems and methods eliminate the need to cross reference several files, write complicated scripts to overlay data, and manually filter for VDJ receptors of interest. As such, the provided systems and methods convert an infeasible task into a fast, easy-to-use workflow.

The provided methods and systems additionally enable a user to export data the user finds particularly useful based on filtering criteria or based on user indications that highlight (e.g., favorite, star, etc.) certain data. In one example, a user may export a file including barcodes associated with clonotypes that pass filtering criteria. The user may import this file including barcodes into a another suitable computing system to explore gene expression differences in the different clonotypes that the user is interested in learning more about, which increases data analysis efficiency by providing one output for the user's desired analyses. Otherwise, the user would have to export individual barcodes, or have to connect information from several output files to narrow down on a barcode list.

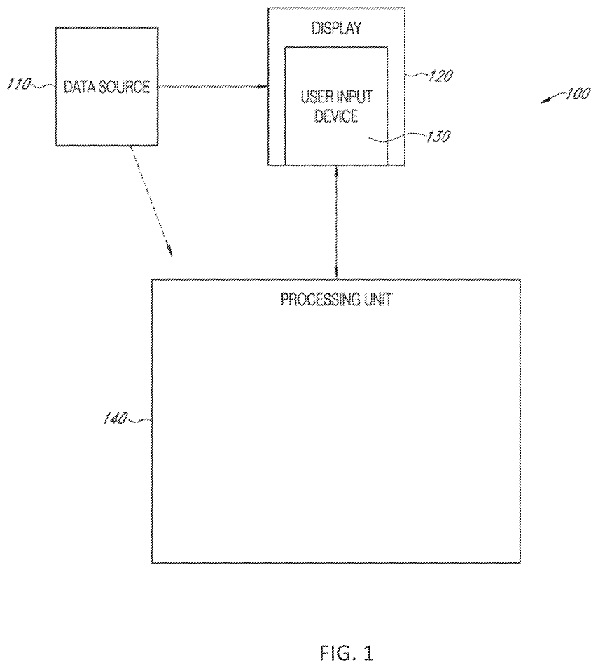

In accordance with various embodiments, FIG. 1 illustrates an interactive visualization system 100. System 100 can comprise a data source 110, a display 120, a user input device 130, and a processor 140. While user input device 130 is shown as part of display 120, it should be understood that these components also can be independent.

Note that all previous discussion of additional features, particularly with regard to the preceding described methods and graphical user interfaces, in accordance with various embodiments, are applicable to the features of the various system embodiments described and contemplated herein.

In accordance with various embodiments, the data source 110 can be configured to obtain a data set comprising B cell receptor and/or T cell receptor data associated with a plurality of cells, e.g., a plurality of immune cells.

In some embodiments, the data set may include sequence data associated with an immune cell of the plurality of immune cells. In some embodiments, the sequence data associated with the immune cell is generated by at least partitioning a reaction mixture, or a portion thereof, into a plurality of partitions, wherein the reaction mixture comprises (i) a plurality of immune cells, (ii) a target antigen, and optionally (iii) a control antigen, wherein the target antigen is operatively coupled to a first reporter oligonucleotide comprising a first reporter barcode sequence, wherein the control antigen is operatively coupled to a second reporter oligonucleotide comprising a second reporter barcode sequence, and wherein the partitioning provides a partition comprising (i) the immune cell, and (ii) a plurality of nucleic acid barcode molecules comprising a partition-specific barcode sequence; using a first analyte comprising a nucleic acid sequence encoding at least a portion of the antigen binding molecule expressed by the immune cell, and a first nucleic acid barcode molecule of the plurality of nucleic acid barcode molecules, to generate a first barcoded polynucleotide comprising (i) a sequence of the first analyte or reverse complement thereof and (ii) the partition-specific barcode sequence or a reverse complement thereof; and determining a sequence of the first barcoded polynucleotide or derivative thereof.

In some embodiments, the provided partition may further include the target antigen. Exemplary target antigens are described herein. The sequence data associated with the immune cell may further include target antigen data. The target antigen data may be generated by using the first reporter oligonucleotide and a second nucleic acid barcode molecule of the plurality of nucleic acid barcode molecules to generate a second barcoded polynucleotide comprising (i) the first reporter barcode sequence or reverse complement thereof and (ii) the partition-specific barcode sequence or a reverse complement thereof; and determining a sequence of the second barcoded polynucleotide or derivative thereof.

In some embodiments, the provided partition may further include the control antigen. Exemplary control antigens are described herein. The sequence data associated with the immune cell may further include control antigen data. The control antigen data may be generated by using the second reporter oligonucleotide and a third nucleic acid barcode molecule of the plurality of nucleic acid barcode molecules to generate a third barcoded polynucleotide comprising (i) the second reporter barcode sequence or reverse complement thereof and (ii) the partition-specific barcode sequence or a reverse complement thereof; and determining a sequence of the third barcoded polynucleotide or derivative thereof.

In some embodiments, the sequence dataset associated with the immune cell may further include data generated by using a second analyte comprising a nucleic acid sequence encoding at least a different portion of the antigen binding molecule expressed by the immune cell and a fourth nucleic acid barcode molecule of the plurality of nucleic acid barcode molecules to generate a fourth barcoded polynucleotide comprising (i) a sequence of the second analyte or reverse complement thereof and (ii) the partition-specific barcode sequence or a reverse complement thereof; and determining a sequence of the fourth barcoded polynucleotide or derivative thereof.

In some embodiments, the first analyte may include one or more of a variable (V) gene segment sequence, a joining (J) gene segment sequence, a diversity (D) gene segment sequence, or a constant (C) gene segment sequence of the antigen binding molecule.

In some embodiments, the second analyte may include one or more of a variable (V) gene segment sequence, a joining (J) gene segment sequence, a diversity (D) gene segment sequence, or a constant (C) gene segment sequence of the antigen binding molecule.

In some embodiments, the first analyte may encode at least a portion of a B cell receptor (BCR) heavy chain. The second analyte may encode at least a portion of a B cell receptor (BCR) light chain of the antigen binding molecule.

In some embodiments, the first analyte may encode at least a portion of a T cell receptor (TCR) alpha chain. The second analyte may encode at least a portion of a T cell receptor (TCR) beta chain of the antigen binding molecule.

In some embodiments, any one or more of the first, second, third, and fourth barcoded polynucleotides may include a unique molecular identifier (UMI) sequence or a reverse complement thereof.

In some embodiments, the sequence data associated with the immune cell includes a UMI Count/antigen, e.g., a target antigen UMI count. The target antigen UMI count may be determined based at least on a quantity of unique molecular identifier (UMI) sequences or reverse complements of unique molecular identifier (UMI) sequences associated with (i) the partition-specific barcode sequence or a reverse complement thereof, and (ii) the first reporter barcode sequence or a reverse complement thereof.

In some embodiments, the sequence data associated with the immune cell includes a control antigen UMI count. The control antigen UMI count may be determined based at least on a quantity of unique molecular identifier (UMI) sequences or reverse complements of unique molecular identifier (UMI) sequences associated with (i) the partition-specific barcode sequence or a reverse complement thereof, and (ii) and the second reporter barcode sequence or a reverse complement thereof.

In some embodiments, the sequence data associated with the immune cell includes an antigen specificity determination. The antigen specificity determination may be based on the target antigen UMI count and the control antigen UMI count.

A target antigen can be, e.g., a user-selected antigen for which binding by an antigen binding molecule, e.g., an antibody, BCR, or antigen binding fragment thereof, is desired. In some embodiments, the target antigen can include a target antigenic peptide, bound to an MHC molecule, to which binding by a TCR or antigen binding fragment thereof is desired. The target antigen can be associated with an infectious agent such as a viral, bacterial, parasitic, protozoal or prion agent. The target antigen may be an antigen associated a viral agent. In these instances, the viral agent may be an influenza virus, a coronavirus, a retrovirus, a rhinovirus, or a sarcoma virus. In other instances, the viral agent may be severe acute respiratory syndrome coronavirus 1 (SARS-CoV-1), a SARS-CoV-2, a Middle East respiratory syndrome coronavirus (MERS-CoV), or human immunodeficiency virus (HIV), influenza, respiratory syncytial virus, or Ebola virus. The target antigen may be an antigen associated with a tumor or a cancer. Antigens associated with a tumor or cancer, include any of epidermal growth factor receptor (EGFR), CD38, platelet-derived growth factor receptor (PDGFR) alpha, insulin growth factor receptor (IGFR), CD20, CD19, CD47, ERBB2IP, TP53, KRAS, MAGEA1, LC3A2, KIAA0368, CADPS2, CTSB or human epidermal growth factor receptor 2 (HER2). The target antigen may be an checkpoint molecule associated with tumors or cancers (e.g., CD38, PD-1, CTLA-4, TIGIT, LAG-3, VISTA, TIM-3), or it may be a cytokine, a GPCR, a cell-based co-stimulatory molecule, a cell-based co-inhibitory molecule, an ion channel, or a growth factor. The target antigen may be associated with a degenerative condition or disease.

In some embodiments, a control antigen is a non-target antigen (e.g., negative control antigen). Non-target antigens (e.g., negative control antigens) may be any antigen to which the antibodies or antigen-binding fragments thereof, would not be expected to bind. In some embodiments, the non-target antigen has been selected such that it is not expected to bind the antibody or antigen-binding fragment thereof. By way of example, the non-target antigen may be any antigen for which a subject (e.g., a human subject) would not be expected to develop an antibody response to or to have antibodies with a specificity for. Such a non-target antigen may be an antigen endogenous to and abundantly expressed in a subject, e.g., a human subject, e.g., human serum albumin (HSA).

In some embodiments wherein the target antigen is a target MHC molecule complex comprising a target antigenic peptide bound to an MHC molecule, the control antigen may be a control MHC molecule complex comprising a control peptide bound to an MHC molecule. The control peptide may be a scrambled peptide, serum albumin peptide, a heteroclitic peptide, or peptide to which immune cells of the sample are naïve. The scrambled peptide may have the same amino acid residue composition as a target antigenic peptide (bound to the first MHC molecule of the target MHC molecule complex), wherein the amino acid residues are presented in a different, e.g., scrambled, order relative to that of the target antigenic peptide. The serum albumin peptide may be a human or mouse serum albumin peptide. The control peptide may be any peptide, e.g., not only a serum albumin peptide, to which the ABMs of the plurality of immune cells would not be expected to bind, e.g., cardiolipin, keyhole limpet hemocyanin, flagellin or insulin. In instances in which the control peptide is a peptide to which ABMs of the plurality of immune cells would not be expected to bind, the control peptide may be a peptide of an abundantly expressed self-antigen of a subject from which the plurality of immune cells had been obtained. In other instances in which the control peptide is a peptide to which ABMs of the plurality of immune cells would not be expected to bind, the control peptide may be a peptide or peptide fragment of an antigen to which the plurality of immune cells are naïve. For example, the control peptide may be a peptide or peptide fragment of an antigen of a virus, e.g. HIV (e.g., TPGPGVRYPL), if the subject from which the plurality of immune cells have been obtained, has not been exposed to the virus, e.g., HIV. For other example, the control peptide may be a heteroclitic peptide. Heteroclitic peptides may include peptides having valine, or leucine or other suitable residues at positions that anchor the peptide to the second MHC molecule, e.g., position 2 and/or a C-terminal residue, but alanine residues at the remaining amino acid positions (e.g., ALAAAAAAV, ATAAAAAAK, AYAAAAAAL, APAAAAAAV or RYAAAAALL). Additional examples of negative control peptides include ASYAAAAV and vaccinia virus peptide TSYKFESV.

Further detail regarding the UMI Count/antigen and the antigen specificity determination is provided below in the Antigen Specificity and UMI count/antigen section.

In general, the data set is contained in a multi-section data file. In at least some examples, the multi-section data file is created by reading the data sets contained in a plurality of discrete output files and writing the discrete data sets to a database (e.g., SQLite) that is then converted (e.g., serialized) into the multi-section data file in one of its sections. During the conversion, one or more aggregate metrics may be calculated and inserted into the database. In another section of the multi-section data file, the raw sequences and alignment information (CIGAR strings) for clonotype chains, exact clonotype chains, and the donor and universal references are written as a concatenated string. Writing this information in the concatenated string optimizes storage space and disregards query speed since the sequence and alignment data are potentially very large, and at least in some aspects, there are not any filters that operate directly on the sequence and alignment data. When the sequence and alignment data is needed, pointers stored in the database may be used to perform random access into the section containing the sequence data.

As such, generating the multi-section data file consolidates multiple discrete data sets in multiple discrete files, that may be generated at different sources, into a single file. The data set in the multi-section data file is therefore faster to query when stored in one place. For instance, the database provides performance, indexing, and an API that is better suited for the filters and other features of the provided systems and methods. Additionally, the discrete files prior to conversion into the multi-section data file are not suited for efficient querying as these discrete files do not contain indexes that would help avoid iteration over large amounts of data to perform filtering. Another advantage of the multi-section data file is that condensing the multiple discrete data sets into a single file makes the consolidated data set more resistant to tampering and easier to share. For example, when considering a multi-file directory where several files represent the data set, a user could intentionally or accidentally move, rename or share a subset of the files, thereby leading to a state where system would not be able to find part of the data or otherwise open the data set.

As stated above, the data set may include UMI counts and/or an antigen specificity determination. The format of the multi-section data file supports queries that operate on a multi-section data file including UMI counts and/or an antigen specificity determination and on a multi-section data file that does not include either. For instance, the UMI count and/or antigen specificity data may be written into separate tables from other data in the database. Storing the antigen specificity data next to the other data makes all of the data efficient to query. Additionally, the provided systems and methods are able to differentiate between a multi-section data file generated with UMI count and/or antigen specificity data, and a multi-section data file generated without either, and modify filtering of the data set and display of the visualization in response. For instance, the systems can detect whether the separate table for the UMI count and/or antigen specificity data exists and perform operations appropriately.

Additionally, as described herein, the multi-section data file may be used to render both a table of information and a visualization. For each of the filters described herein, the shape of the data after filtering for the table of information is different from the shape of the data after filtering for the visualization. The difference in data shapes between the table and the visualization enables the table and the visualization to be rendered to the screen efficiently. In an example, the provided system runs separate database queries against the multi-section data file and converts the returned data into the appropriate shapes while the views of the table and the visualization are kept in sync with the filtering.

As described herein, the multi-section data file may be used to render a visualization of sequences of each of two paired chains for a cell receptor. While data for each of the two single chains composing the paired chains may have existed, utilizing the existing independent queries for the single chains to build the paired chain sequence view would have had less than desired performance and possibly have led to incorrect output. Instead, the provided systems and methods include database queries that return the paired chains efficiently in a single request.

### Clonotype Distribution View

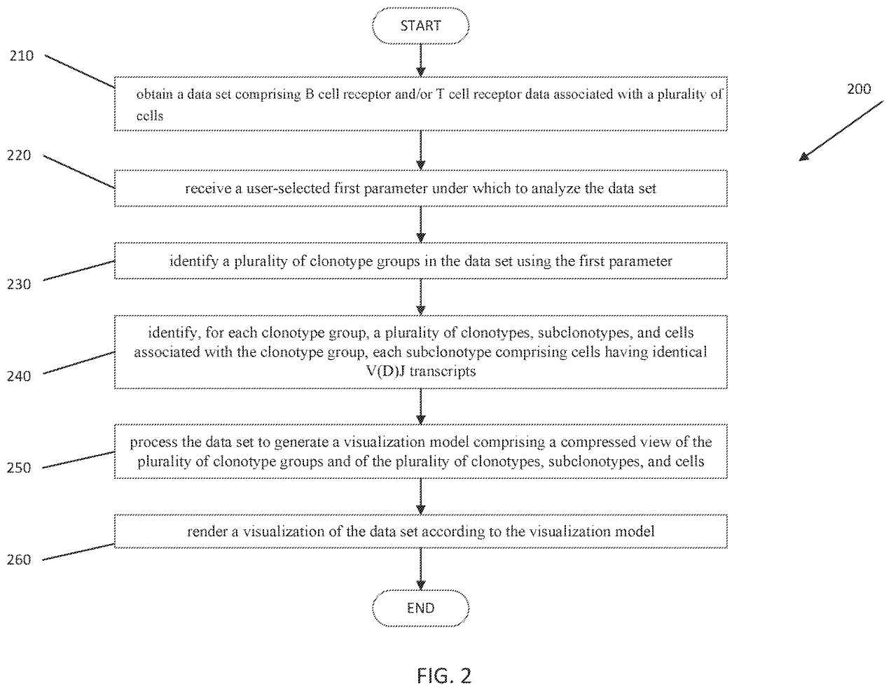

In accordance with various embodiments, the processor 140 can render a visualization of the data set in the form of a plot representing a plurality of clonotype groups and plurality of clonotypes and, optionally, subclonotypes. To render the visualization, in some embodiments, the user input device 130 can be configured to receive a user-selected first parameter under which to analyze the data set. In accordance with various embodiments, the processor 140 can be configured to implement a method. The method can comprise: (a) identifying a plurality of clonotype groups in the data set using the first parameter; (b) for a clonotype group, identifying a plurality of clonotypes and, optionally, subclonotypes associated with the clonotype group, each subclonotype comprising a subset of the cells having identical V(D)J transcripts, and (c) processing the data set to generate a visualization model comprising a compressed view of the plurality of clonotype groups and of the plurality of clonotypes and, optionally, subclonotypes. The method can be similar to method 200 described herein with respect to FIG. 2.

In accordance with various embodiments, the display 120 can be configured to render a visualization of the data set according to the visualization model. In these various embodiments, the visualization model may be a plot of shapes representative of the clonotype groups in the data set.

In accordance with various embodiments, the first parameter can comprise one or more members selected from the group consisting of: isotype, mutation rate, mutation location, presence of specified amino acids, absence of specified amino acids, quantity of specified amino acids, location of specified amino acids, presence of specified nucleic acid motifs, absence of specified nucleic acid motifs, quantity of specified nucleic acid motifs, location of specified nucleic acid motifs, gene expression, surface protein count, surface antigen count, intracellular protein count, intracellular antigen count, reads for each cell, unique molecular identifiers for each cell, quality control information, user-specified metadata about a sequence from a cell barcode, user-specified metadata about a sequence from a clonotype group, antigen specificity information, donor information, and sample information.

In accordance with various embodiments, the user input device can be configured to receive a user-selected second parameter under which to analyze the data set. The processor can be configured to perform (b), at least in part, by identifying the plurality of clonotypes and, optionally, subclonotypes based on the second parameter. The second parameter can comprise one or more members selected from the group consisting of: isotype, mutation rate, mutation location, presence of specified amino acids, absence of specified amino acids, quantity of specified amino acids, location of specified amino acids, presence of specified nucleic acid motifs, absence of specified nucleic acid motifs, quantity of specified nucleic acid motifs, location of specified nucleic acid motifs, gene expression, surface protein count, surface antigen count, intracellular protein count, intracellular antigen count, reads for each cell, unique molecular identifiers for each cell, quality control information, user-specified metadata about a sequence from a cell barcode, user-specified metadata about a sequence from a subclonotype, antigen specificity information, donor information, and sample information.

In accordance with various embodiments, the processor can be configured to perform (c), at least in part, by generating a plurality of shapes. Each shape can be associated with a clonotype group, or individual clonotype. A largest shape can be placed near a center of the visualization model. A next largest shape can be placed radiating out from the center of the visualization model. This can be repeated until all shapes have been placed. The shapes can be placed at a location that minimizes empty space within the visualization model. For example, each shape can be randomly placed at a plurality of locations and the amount of empty space associated with that location can be measured. The location that is associated with the minimum amount of empty space can be chosen as the location at which the shape is placed. Each shape can be placed at least about 1, 2, 3, 4, 5, 6, 7, 8, 9, 10, 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, 1,000,000, or more locations. Each shape can be placed at most about 1,000,000, 900,000, 800,000, 700,000, 600,000, 500,000, 400,000, 300,000, 200,000, 100,000, 90,000, 80,000, 70,000, 60,000, 50,000, 40,000, 30,000, 20,000, 10,000, 9,000, 8,000, 7,000, 6,000, 5,000, 4,000, 3,000, 2,000, 1,000, 900, 800, 700, 600, 500, 400, 300, 200, 100, 90, 80, 70, 60, 50, 40, 30, 20, 10, 9, 8, 7, 6, 5, 4, 3, 2, or 1 locations. Each shape can be placed at a number of locations that is within a range defined by any two of the preceding values. The plurality of shapes can be placed at a location determined at least in part by Lloyd's algorithm, Voronoi iteration, or Voronoi relaxation. A geometric form of each shape can be generated by minimizing empty space within the visualization model.

In accordance with various embodiments, the method can further comprise coloring each shape based on one or more members selected from the group consisting of: isotype, mutation rate, mutation location, presence of specified amino acids, absence of specified amino acids, quantity of specified amino acids, location of specified amino acids, presence of specified nucleic acid motifs, absence of specified nucleic acid motifs, quantity of specified nucleic acid motifs, location of specified nucleic acid motifs, gene expression, surface protein count, surface antigen count, intracellular protein count, intracellular antigen count, reads for each cell, unique molecular identifiers for each cell, quality control information, user-specified metadata about a sequence from a cell barcode, user-specified metadata about a sequence from a clonotype group, antigen specificity information, donor information, and sample information.

In accordance with various embodiments, the processor can be configured to perform (c), at least in part, by placing each cell, subclonotype, and clonotype associated with a specific clonotype group in the shape associated with the clonotype group. For example, a largest clonotype can be placed near a center of the shape. A next largest clonotype can be placed radiating out from the center of the shape. This may be repeated until all clonotypes have been placed. The clonotypes can be placed at a location that minimizes empty space within the shape. The clonotypes can be placed at a location determined at least in part by Lloyd's algorithm, Voronoi iteration, or Voronoi relaxation.

It should be understood that one or more dots that make up the shapes, discussed herein, can represent a cell. These cells within the shape can belong to a given clonotype. This shape can be grouped together with other clonotypes (sharing a common shape) to represent a clonotype group, where clonotypes can be grouped according to a user-defined criteria (for example, particular isotype, or other characteristic).

In accordance with various embodiments, the method can further comprise coloring each subclonotype based on one or more members selected from the group consisting of: isotype, mutation rate, mutation location, presence of specified amino acids, absence of specified amino acids, quantity of specified amino acids, location of specified amino acids, presence of specified nucleic acid motifs, absence of specified nucleic acid motifs, quantity of specified nucleic acid motifs, location of specified nucleic acid motifs, gene expression, surface protein count, surface antigen count, intracellular protein count, intracellular antigen count, reads for each cell, unique molecular identifiers for each cell, quality control information, user-specified metadata about a sequence from a cell barcode, user-specified metadata about a sequence from a subclonotype, antigen specificity information, donor information, and sample information.

In accordance with various embodiments, the user input device 130 can be configured to receive a user command to display information associated with the one or more cells. The method can further comprise displaying the information associated with the one or more cells. The information can comprise one or more members selected from the group consisting of: gene expression counts, antibody protein counts, surface protein counts, donor identity, sample origin information, cell origin information, cell barcode information, mutation percentage, previously identified sequence metadata, functional assay performance metadata, number of targetable unique molecular identifiers for cloning, single cell summary statistics, isotype, mutation rate, mutation location, presence of specified amino acids, absence of specified amino acids, quantity of specified amino acids, location of specified amino acids, presence of specified nucleic acid motifs, absence of specified nucleic acid motifs, quantity of specified nucleic acid motifs, location of specified nucleic acid motifs, gene expression, surface protein count, surface antigen count, intracellular protein count, intracellular antigen count, reads for each cell, unique molecular identifiers for each cell, quality control information, user-specified metadata about a sequence from a cell barcode, user-specified metadata about a sequence from a clonotype group, antigen specificity information, donor information, and sample information. The single cell summary statistics can comprise means, medians, or percentiles of unique molecular identifiers for a given feature or a given chain of the clonotype, on a per-cell or aggregated basis.

In accordance with various embodiments, the user input device 130 can be configured to receive a user command to dynamically update the visualization model. The method can further comprise dynamically updating the visualization model. The user command can comprise a command to zoom in on a portion of the visualization, zoom out from portion of the visualization, or pan from a first portion of the visualization to the second portion of the visualization. The method can further comprise zooming in on the portion, zooming out from the portion, or panning from the first portion to the second portion. The user command can comprise a command to highlight or grey out a portion of the visualization. The method can further comprise highlighting or greying out the portion.

In accordance with various embodiments, processor 140 of system 100 of FIG. 1 can be communicatively connected to data source 110 (see dotted line in FIG. 1), display 120, and/or user input device 130. In various embodiments, processor 140 can include various engines configured to carry out the functionality of processor 140. It should be appreciated that each component (e.g., engine, module, unit, etc.) depicted as part of system 100 (and described herein) can be implemented as hardware, firmware, software, or any combination thereof.

In various embodiments, processor 140 can be implemented as an integrated instrument system assembly with any of data source 110, display 120, and user input device 130. That is, any combination of processor 140, data source 110, display 120, and user input device 130 can be housed in the same housing assembly and communicate via conventional device/component connection means (e.g. serial bus, optical cabling, electrical cabling, etc.).

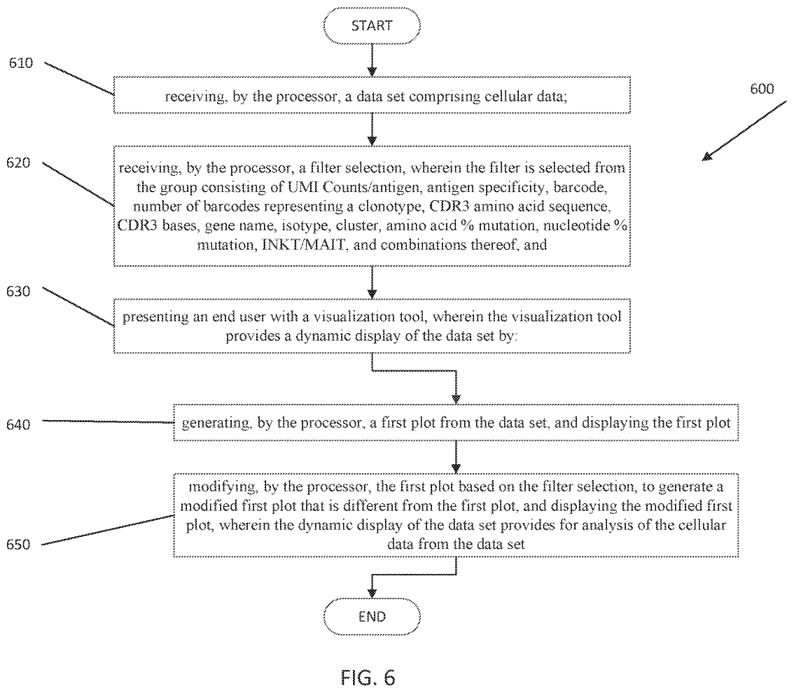

In various embodiments, processor 140 can be implemented as a standalone computing device (as shown in FIG. 6) that can be communicatively connected to the data source 110 (and likewise display 120 and user input device 130) via an optical, serial port, network or modern connection. For example, the processor 140 can be connected via a LAN or WAN connection that allows for the transmission of data to and from the data source 110, and likewise display 120 and user input device 130.

In various embodiments, the functions of processor 140 can be implemented on a distributed network of shared computer processing resources (such as a cloud computing network) that is communicatively connected to the data source 110 via a WAN (or equivalent) connection. For example, the functionalities of processor 140 can be divided up to be implemented in one or more computing nodes on a cloud processing service such as AMAZON WEB SERVICES™.

Within the processor 140, any internal engines can be implemented as separate engines or a single multi-functional engine. As such, FIG. 1 simply provides one example implementation of a system in accordance with various embodiments, and should be not be read to limit the interchangeability, interoperability and/or functionality of all the components therein.

In accordance with various embodiments, FIG. 2 illustrates a method 200. Method 200 can comprise a first operation 210, a second operation 220, a third operation 230, a fourth operation 240, a fifth operation 250, and a sixth operation 260. In some embodiments, method 200 comprises first operation 210, fifth operation 250, and sixth operation 260.

At 210, a data set comprising B cell receptor and/or T cell receptor data associated with a plurality of cells is obtained. In accordance with various embodiments, the B cell receptor and/or T cell receptor data associated with a plurality of cells can include clonotype and optionally subclonotype information. In accordance with various embodiments, the clonotype and subclonotype information can be determined as described in WO2021173502, which is hereby incorporated by reference.

In accordance with various embodiments, at 220, a user-selected first parameter under which to analyze the data set is received.

In accordance with various embodiments, at 230, a plurality of clonotype groups in the data set is identified using the first parameter.

In accordance with various embodiments, at 240, for each clonotype group, a plurality of clonotypes, subclonotypes, and cells associated with the clonotype group are identified, each subclonotype comprising cells having identical V(D)J transcripts.

In accordance with various embodiments, at 250, the data set is processed to generate a visualization model comprising a compressed view of the plurality of clonotype groups and of the plurality of clonotypes, subclonotypes, and cells.

In accordance with various embodiments, at 260, a visualization of the data set is rendered according to the visualization model.

In accordance with various embodiments, the first parameter can comprise one or more members selected from the group consisting of: isotype, mutation rate, mutation location, presence of specified amino acids, absence of specified amino acids, quantity of specified amino acids, location of specified amino acids, presence of specified nucleic acid motifs, absence of specified nucleic acid motifs, quantity of specified nucleic acid motifs, location of specified nucleic acid motifs, gene expression, surface protein count, surface antigen count, intracellular protein count, intracellular antigen count, reads for each cell, unique molecular identifiers for each cell, quality control information, user-specified metadata about a sequence from a cell barcode, user-specified metadata about a sequence from a clonotype group, antigen specificity information, donor information, and sample information.

In accordance with various embodiments, the method 200 can further comprise receiving a user-selected second parameter under which to analyze the data set. Operation 240 can comprise identifying the plurality of subclonotypes based on the second parameter. The second parameter can comprise one or more members selected from the group consisting of: isotype, mutation rate, mutation location, presence of specified amino acids, absence of specified amino acids, quantity of specified amino acids, location of specified amino acids, presence of specified nucleic acid motifs, absence of specified nucleic acid motifs, quantity of specified nucleic acid motifs, location of specified nucleic acid motifs, gene expression, surface protein count, surface antigen count, intracellular protein count, intracellular antigen count, reads for each cell, unique molecular identifiers for each cell, quality control information, user-specified metadata about a sequence from a cell barcode, user-specified metadata about a sequence from a subclonotype, antigen specificity information, donor information, and sample information.

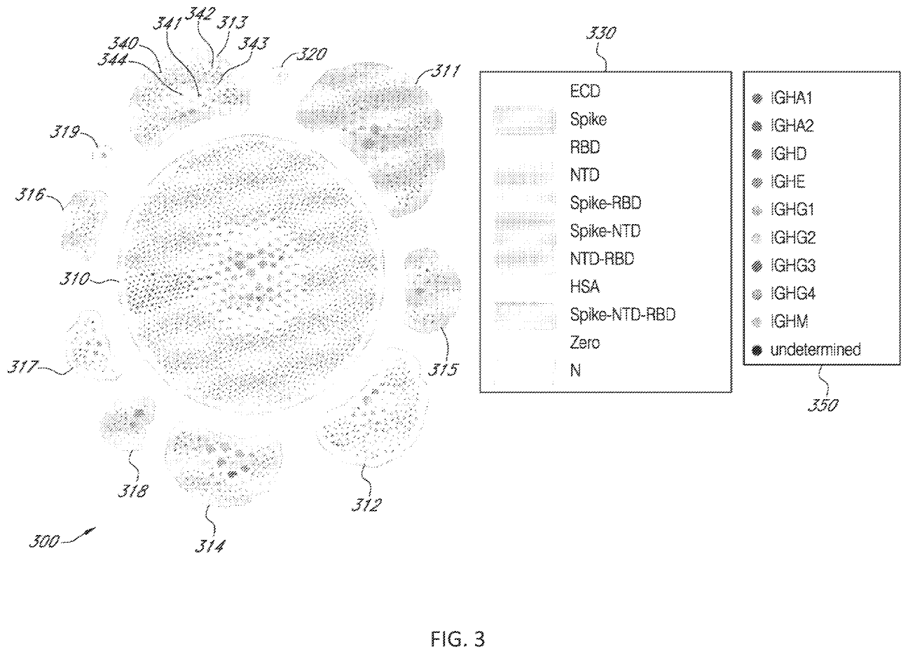

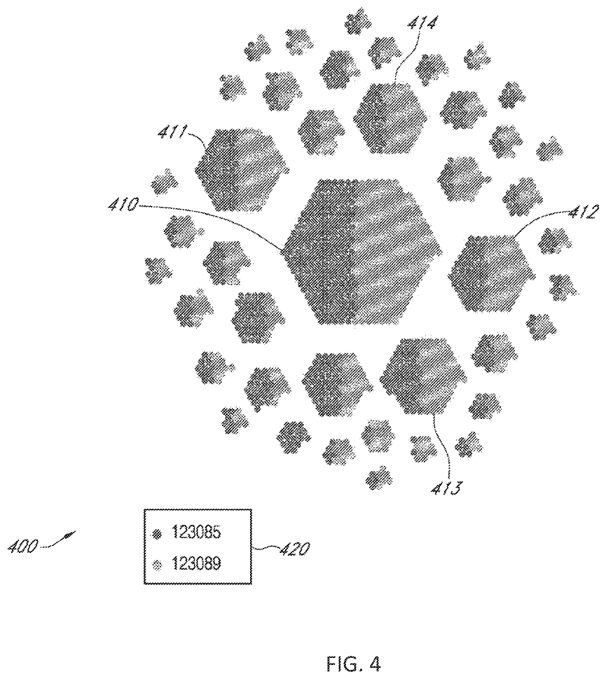

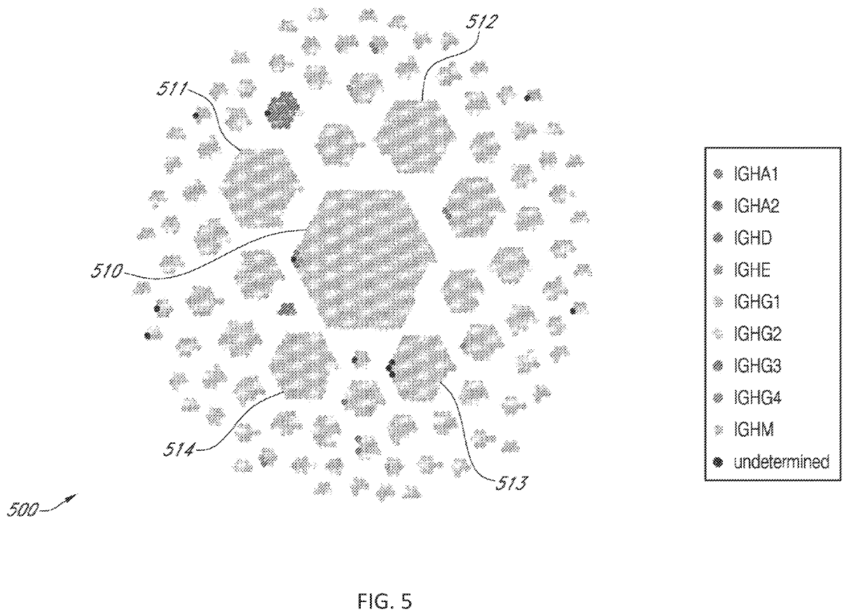

In accordance with various embodiments, operation 250 can comprise generating a plurality of shapes, each shape associated with a clonotype group (see FIG. 3) (see FIGS. 4 and 5 for examples of clonotype and subclonotype visualization within a clonotype group). Operation 250 can comprise: (i) placing a largest shape near a center of the visualization model; (ii) placing a next largest shape radiating out from the center of the visualization model; and (iii) repeating (ii) until all shapes have been placed. The shapes can be placed at a location that minimizes empty space within the visualization model. For example, each shape can be randomly placed at a plurality of locations and the amount of empty space associated with that location can be measured. The location that is associated with the minimum amount of empty space can be chosen as the location at which the shape is placed. Each shape can be placed at least about 1, 2, 3, 4, 5, 6, 7, 8, 9, 10, 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, 1,000,000, or more locations. Each shape can be placed at most about 1,000,000, 900,000, 800,000, 700,000, 600,000, 500,000, 400,000, 300,000, 200,000, 100,000, 90,000, 80,000, 70,000, 60,000, 50,000, 40,000, 30,000, 20,000, 10,000, 9,000, 8,000, 7,000, 6,000, 5,000, 4,000, 3,000, 2,000, 1,000, 900, 800, 700, 600, 500, 400, 300, 200, 100, 90, 80, 70, 60, 50, 40, 30, 20, 10, 9, 8, 7, 6, 5, 4, 3, 2, or 1 locations. Each shape can be placed at a number of locations that is within a range defined by any two of the preceding values. The plurality of shapes can be placed at a location determined at least in part by Lloyd's algorithm, Voronoi iteration, or Voronoi relaxation. A geometric form of each shape can be generated by minimizing empty space within the visualization model.

In accordance with various embodiments, the method 200 can further comprise coloring each shape based on one or more members selected from the group consisting of: isotype, mutation rate, mutation location, presence of specified amino acids, absence of specified amino acids, quantity of specified amino acids, location of specified amino acids, presence of specified nucleic acid motifs, absence of specified nucleic acid motifs, quantity of specified nucleic acid motifs, location of specified nucleic acid motifs, gene expression, surface protein count, surface antigen count, intracellular protein count, intracellular antigen count, reads for each cell, unique molecular identifiers for each cell, quality control information, user-specified metadata about a sequence from a cell barcode, user-specified metadata about a sequence from a clonotype group, antigen specificity information, donor information, and sample information.

In accordance with various embodiments, operation 250 can comprise placing each clonotype associated with a specific clonotype group in the shape associated with the clonotype group. Operation 250 can comprise: for each shape associated with a specific clonotype group: (iv) placing a largest clonotype near a center of the shape; (v) placing a next largest clonotype radiating out from the center of the shape; and (vi) repeating (v) until all clonotype have been placed. The shapes can be placed at a location that minimizes empty space within the shape. The shapes can be placed at a location determined at least in part by Lloyd's algorithm, Voronoi iteration, or Voronoi relaxation.

In accordance with various embodiments, the method 200 can further comprise coloring each clonotype (or subclonotype(s) within a clonotype) based on one or more members selected from the group consisting of: isotype, mutation rate, mutation location, presence of specified amino acids, absence of specified amino acids, quantity of specified amino acids, location of specified amino acids, presence of specified nucleic acid motifs, absence of specified nucleic acid motifs, quantity of specified nucleic acid motifs, location of specified nucleic acid motifs, gene expression, surface protein count, surface antigen count, intracellular protein count, intracellular antigen count, reads for each cell, unique molecular identifiers for each cell, quality control information, user-specified metadata about a sequence from a cell barcode, user-specified metadata about a sequence from a clonotype (or subclonotype), antigen specificity information, donor information, and sample information.

In accordance with various embodiments, the method 200 can further comprise receiving a user command to display information associated with one or more cells. The method 200 can further comprise displaying the information associated with the one or more cells. The information can comprise one or more members selected from the group consisting of: gene expression counts, antibody protein counts, surface protein counts, donor identity, sample origin information, cell origin information, cell barcode information, mutation percentage, previously identified sequence metadata, functional assay performance metadata, number of targetable unique molecular identifiers for cloning, and single cell summary statistics. The single cell summary statistics can comprise means, medians, or percentiles of unique molecular identifiers for a given feature or a given chain of the clonotype, on a per-cell or aggregated basis.

In accordance with various embodiments, the method 200 can further comprise receiving a user command to dynamically update the visualization model and dynamically updating the visualization model. The user command can comprise a command to zoom in on a portion of the visualization, zoom out from portion of the visualization, or pan from a first portion of the visualization to the second portion of the visualization. The method 200 can further comprise zooming in on the portion, zooming out from the portion, or panning from the first portion to the second portion. The user command can comprise a command to highlight or grey out a portion of the visualization. The method 200 can further comprise highlighting or greying out the portion.

Referring to FIG. 3, a first example visualization 300 is provided, in accordance with various embodiments. It should be noted that many details about the display features, fields, parameters, customizations, etc. are discussed below as opposed to this discussion of the visualizations of FIGS. 3-5. It should be understood, however, that while many of these details are discussed below rather than here, the display features, fields, parameters, customizations, etc., and the associated descriptions are relevant to all embodiments herein and can be implemented in any combination as per user need.

Returning to the discussion of FIG. 3, the visualization 300 can display a plurality of clonotype groups. For example, as shown in FIG. 3, the visualization 300 can display first, second, third, fourth, fifth, sixth, seventh, eighth, ninth, tenth, and eleventh clonotype groups 310, 311, 312, 313, 314, 315, 316, 317, 318, 319, and 320, respectively. As shown in FIG. 3, the clonotype groups are numbered from largest to smallest, with clonotype group 310 the largest, clonotype group 311 the next largest, and so on. Although depicted as display eleven clonotype groups in FIG. 3, the visualization 300 can display any number of clonotype groups. For example, the visualization 300 can display at least about 1, 2, 3, 4, 5, 6, 7, 8, 9, 10, 11, 12, 13, 14, 15, 16, 17, 18, 19, 20, 25, 30, 35, 40, 45, 50, or more clonotype groups. The visualization 300 can display at most about 50, 45, 40, 35, 30, 25, 20, 19, 18, 17, 16, 15, 14, 13, 12, 11, 10, 9, 8, 7, 6, 5, 4, 3, 2, or 1 clonotype groups. The visualization 300 can display a number of clonotype groups that is within a range defined by any two of the preceding values.

In accordance with various embodiments, the clonotype groups can be determined based upon the first parameter described herein. As shown in FIG. 3, clonotype group 310 is identified according to ability to bind the SARS-CoV-2 ECD protein, clonotype group 311 is identified according to ability to bind the SARS-CoV-2 Spike protein, clonotype group 312 is identified according to ability to bind the SARS-CoV-2 RBD protein, clonotype group 313 is identified according to ability to bind the SARS-CoV-2 NTD protein, clonotype group 314 is identified according to ability to bind the SARS-CoV-2 Spike protein and the SARS-CoV-2 RBD protein, clonotype group 315 is identified according to ability to bind the SARS-CoV-2 Spike protein and the SARS-CoV-2 NTD protein, clonotype group 316 is identified according to ability to bind the SARS-CoV-2 NTD protein and the SARS-CoV-2 RBD protein, clonotype group 317 is identified according to ability to bind the SARS-CoV-2 HSA protein, clonotype group 318 is identified according to ability to bind the SARS-CoV-2 Spike protein, the SARS-CoV-2 NTD protein, and the SARS-CoV-2 RBD protein, clonotype group 319 is identified according to the lack of ability to bind any of the previous proteins.

In accordance with various embodiments, the clonotype groups can be colored as described herein with respect to FIGS. 1 and 2. The visualization 300 can display a clonotype group legend 330 showing a correspondence between the color and the first parameter.

In accordance with various embodiments, a clonotype group can comprise a plurality of clonotypes and, optionally, subclonotypes within a clonotype of the clonotype group. For example, as shown in FIG. 3, clonotype group 313 can comprise first, second, third, fourth, and fifth clonotypes 340, 341, 342, 343, 344 respectively (as well as other clonotypes not specifically labeled in FIG. 3). Although depicted as comprising five clonotype in FIG. 3, each clonotype group can comprise any number of clonotypes. For example, each clonotype group can comprise at least about 1, 2, 3, 4, 5, 6, 7, 8, 9, 10, 11, 12, 13, 14, 15, 16, 17, 18, 19, 20, 25, 30, 35, 40, 45, 50, or more clonotypes. Each clonotype group can comprise at most about 50, 45, 40, 35, 30, 25, 20, 19, 18, 17, 16, 15, 14, 13, 12, 11, 10, 9, 8, 7, 6, 5, 4, 3, 2, or 1 clonotypes. Each clonotype group can comprise a number of clonotypes that is within a range defined by any two of the preceding values.

In accordance with various embodiments, the clonotypes can be colored as described herein with respect to FIGS. 1 and 2. The visualization 300 can display a clonotype legend 350 showing a correspondence between the color and the second parameter.

In accordance with various embodiments, the visualization 300 can include a command line (not shown in FIG. 3) that can be used for accepting a user input, in accordance with various embodiments. That user input can be, for example, a file path to a dataset, and additional optional parameters for customizing the output in visualization 300. Specifying data sets can be done various ways including, for example, on the command line (as illustrated) for via a supplementary metadata file. The command line can include BCR, TCR, and CDR3 parameters. Based on this example command line entry, the output visualization would exhibit all clonotypes in which at least one chain has the given CDR3 sequence. The output can be in a compressed view (e.g., streamlined visualization of query results to include essential information for specific analytical purposes).

Referring to FIG. 4, a second example visualization 400 is provided, in accordance with various embodiments. As shown in FIG. 4, the visualization 400 can display first, second, third, fourth, and fifth clonotypes 410, 411, 412, 413 and 414, respectively (as well as other clonotypes not labeled in FIG. 4). As depicted in FIG. 4, a clonotype may comprise cells that are visually differentiated (e.g., differentiated by color) according to a user defined parameter, e.g., sample of origin. The visualization can display a parameter legend 420 of the visual differentiation by user defined criteria.

Referring to FIG. 5, a third example visualization 500 is provided, in accordance with various embodiments. As shown in FIG. 5, the visualization 500 can display first, second, third, fourth, and fifth clonotypes 510, 511, 512, 513 and 514, respectively (as well as other clonotype groups not labeled in FIG. 5). As depicted in FIG. 5, a clonotype may comprise cells that are visually differentiated (e.g., differentiated by color) according to a user defined parameter, e.g., Ig isotype. The visualization 500 can display a parameter legend 520 of the visual differentiation by user defined criteria.

For more detail regarding customization of visualizations, in accordance with various embodiments, refer to the Additional Features of Clonotype Data Visualization section below for detailed discussion. It should be noted that the various parameters, variables, fields, values, filters, etc. discussed in detail herein are independent and interchangeable in any contemplated fashion or combination. Moreover, the various parameters, variables, fields, values, filters, etc. discussed in detail herein are applicable to any and all the various embodiments discussed or contemplated herein.

### Sequence View

A clonotype can be defined by not just the individual CDR3s, but the pairs of CDR3s among the cell receptors. For example, in the case of T-cells a clonotype can be defined by the CDR3 from the alpha chain and the CDR3 from the beta chain. In the case of B-cells, a clonotype can be defined by the CDR3 from the heavy chain and the CDR3 from the light chain. It is valuable to researchers (e.g., immunology researchers) to be able to view visualizations of both paired chains side by side, and in some instances on the screen at the same time, rather than having to click between the two chains. For instance, the specificity to an antigen is a result of the antigen binding regions of the two chains combined together. Individual chains cannot bind to an antigen on their own. As such, when researchers are thinking about the next therapeutic target, they need to consider the property of both chains. Accordingly, in addition to the clonotype distribution view, the data set may also be rendered as a visualization showing sequences of cell receptors included in a clonotype group. For example, a visualization of sequences of each of two paired chains for a cell receptor may be rendered. In various embodiments, a user may navigate to the sequence view from the clonotype distribution view by clicking on a clonotype group listed in the table of clonotype groups displayed with the clonotype distribution plot.

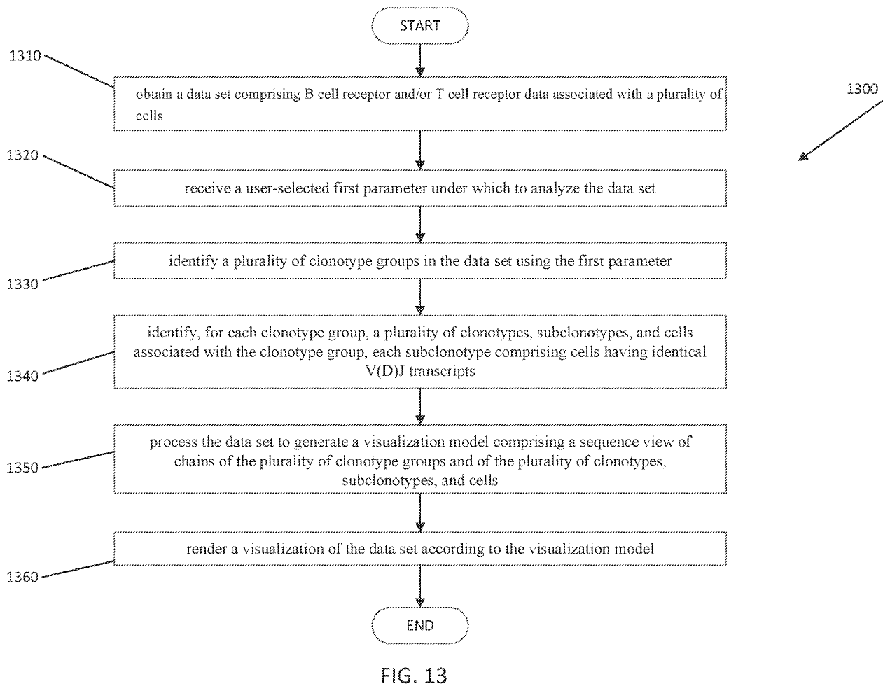

In accordance with various embodiments, the processor 140 can be configured to implement a method. The method can comprise: (a) identifying a plurality of clonotype groups in the data set using the first parameter; (b) for a clonotype group, identifying a plurality of clonotypes and, optionally, subclonotypes associated with the clonotype group, each subclonotype comprising a subset of the cells having identical V(D)J transcripts, and (c) processing the data set to generate a visualization model comprising a sequence view of chains of the plurality of clonotype groups and of the plurality of clonotypes and, optionally, subclonotypes. The method can be similar to method 1300 described herein with respect to FIG. 13.

In accordance with various embodiments, the display 120 can be configured to render a visualization of the data set according to the visualization model. In these various embodiments, the visualization model may be a visualization of one or more sequences of the clonotype groups in the data set.

In accordance with various embodiments, the first parameter can comprise one or more members selected from the group consisting of: isotype, mutation rate, mutation location, presence of specified amino acids, absence of specified amino acids, quantity of specified amino acids, location of specified amino acids, presence of specified nucleic acid motifs, absence of specified nucleic acid motifs, quantity of specified nucleic acid motifs, location of specified nucleic acid motifs, gene expression, surface protein count, surface antigen count, intracellular protein count, intracellular antigen count, reads for each cell, unique molecular identifiers for each cell, quality control information, user-specified metadata about a sequence from a cell barcode, user-specified metadata about a sequence from a clonotype group, antigen specificity information, donor information, and sample information.

In accordance with various embodiments, the user input device can be configured to receive a user-selected second parameter under which to analyze the data set. The processor can be configured to perform (b), at least in part, by identifying the plurality of clonotypes and, optionally, subclonotypes based on the second parameter. The second parameter can comprise one or more members selected from the group consisting of: isotype, mutation rate, mutation location, presence of specified amino acids, absence of specified amino acids, quantity of specified amino acids, location of specified amino acids, presence of specified nucleic acid motifs, absence of specified nucleic acid motifs, quantity of specified nucleic acid motifs, location of specified nucleic acid motifs, gene expression, surface protein count, surface antigen count, intracellular protein count, intracellular antigen count, reads for each cell, unique molecular identifiers for each cell, quality control information, user-specified metadata about a sequence from a cell barcode, user-specified metadata about a sequence from a subclonotype, antigen specificity information, donor information, and sample information.

In accordance with various embodiments, the processor can be configured to perform (c), at least in part, by generating sets of indicia corresponding to chains of a cell receptor.

In accordance with various embodiments, FIG. 13 illustrates a method 1300. Method 1300 can comprise a first operation 1310, a second operation 1320, a third operation 1330, a fourth operation 1340, a fifth operation 1350, and a sixth operation 1360. In some embodiments, method 1300 comprises first operation 1310, fifth operation 1350, and sixth operation 1360.

At 1310, a data set comprising B cell receptor and/or T cell receptor data associated with a plurality of cells is obtained. In accordance with various embodiments, the B cell receptor and/or T cell receptor data associated with a plurality of cells can include clonotype and optionally subclonotype information. In accordance with various embodiments, the clonotype and subclonotype information can be determined as described in WO2021173502, which is hereby incorporated by reference.

In accordance with various embodiments, at 1320, a user-selected first parameter under which to analyze the data set is received.

In accordance with various embodiments, at 1330, a plurality of clonotype groups in the data set is identified using the first parameter.

In accordance with various embodiments, at 1340, for each clonotype group, a plurality of clonotypes, subclonotypes, and cells associated with the clonotype group are identified, each subclonotype comprising cells having identical V(D)J transcripts.

In accordance with various embodiments, at 1350, the data set is processed to generate a visualization model comprising a sequence view of chains of the plurality of clonotype groups and of the plurality of clonotypes, subclonotypes, and cells.

In accordance with various embodiments, at 1360, a visualization of the data set is rendered according to the visualization model.

In accordance with various embodiments, the first parameter can comprise one or more members selected from the group consisting of: isotype, mutation rate, mutation location, presence of specified amino acids, absence of specified amino acids, quantity of specified amino acids, location of specified amino acids, presence of specified nucleic acid motifs, absence of specified nucleic acid motifs, quantity of specified nucleic acid motifs, location of specified nucleic acid motifs, gene expression, surface protein count, surface antigen count, intracellular protein count, intracellular antigen count, reads for each cell, unique molecular identifiers for each cell, quality control information, user-specified metadata about a sequence from a cell barcode, user-specified metadata about a sequence from a clonotype group, antigen specificity information, donor information, and sample information.

In accordance with various embodiments, the method 1300 can further comprise receiving a user-selected second parameter under which to analyze the data set. Operation 1340 can comprise identifying the plurality of subclonotypes based on the second parameter. The second parameter can comprise one or more members selected from the group consisting of: isotype, mutation rate, mutation location, presence of specified amino acids, absence of specified amino acids, quantity of specified amino acids, location of specified amino acids, presence of specified nucleic acid motifs, absence of specified nucleic acid motifs, quantity of specified nucleic acid motifs, location of specified nucleic acid motifs, gene expression, surface protein count, surface antigen count, intracellular protein count, intracellular antigen count, reads for each cell, unique molecular identifiers for each cell, quality control information, user-specified metadata about a sequence from a cell barcode, user-specified metadata about a sequence from a subclonotype, antigen specificity information, donor information, and sample information.

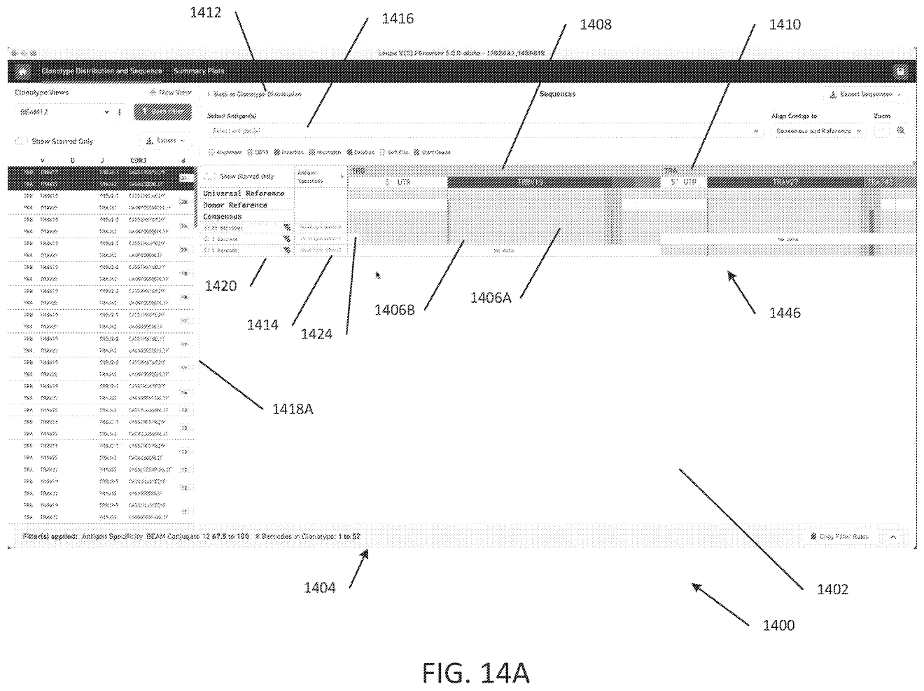

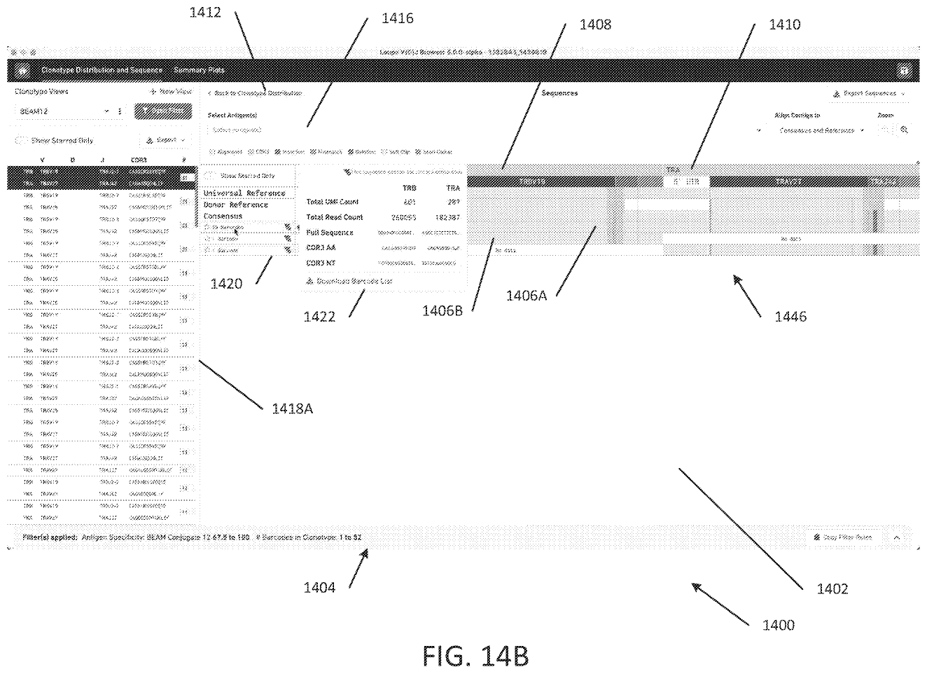

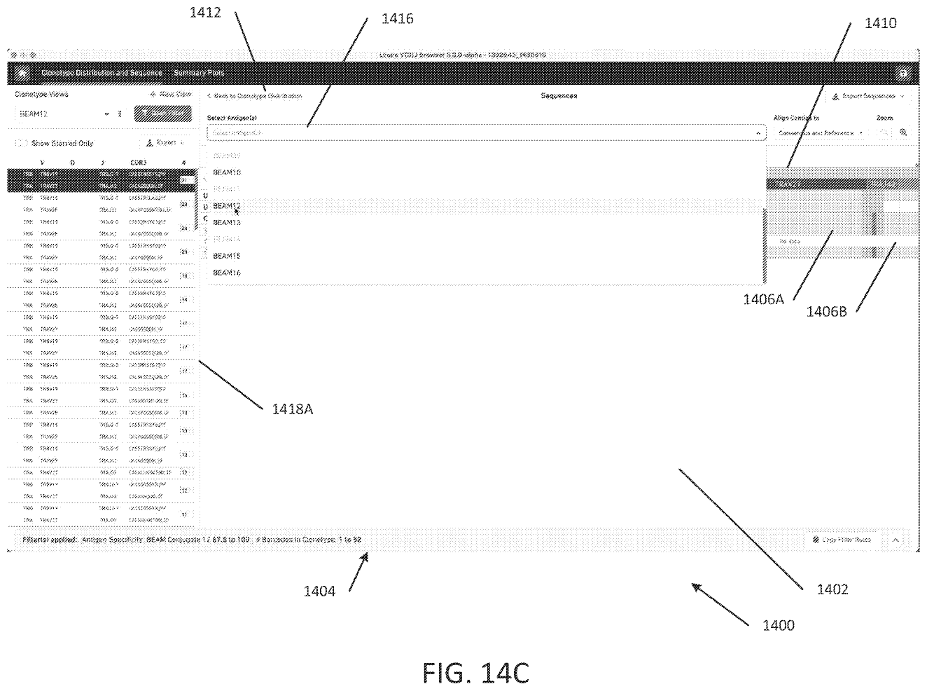

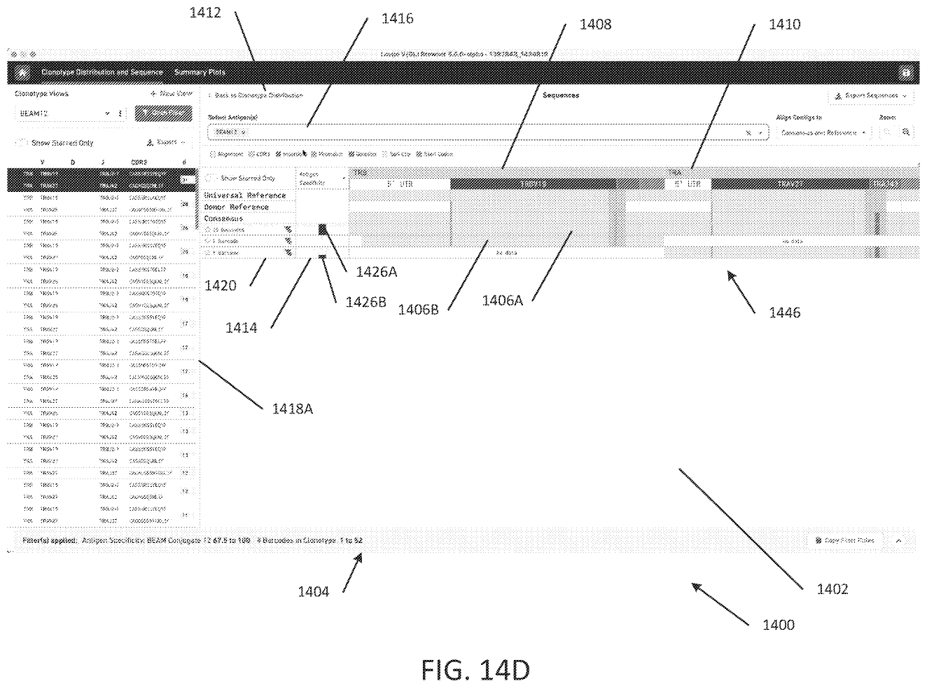

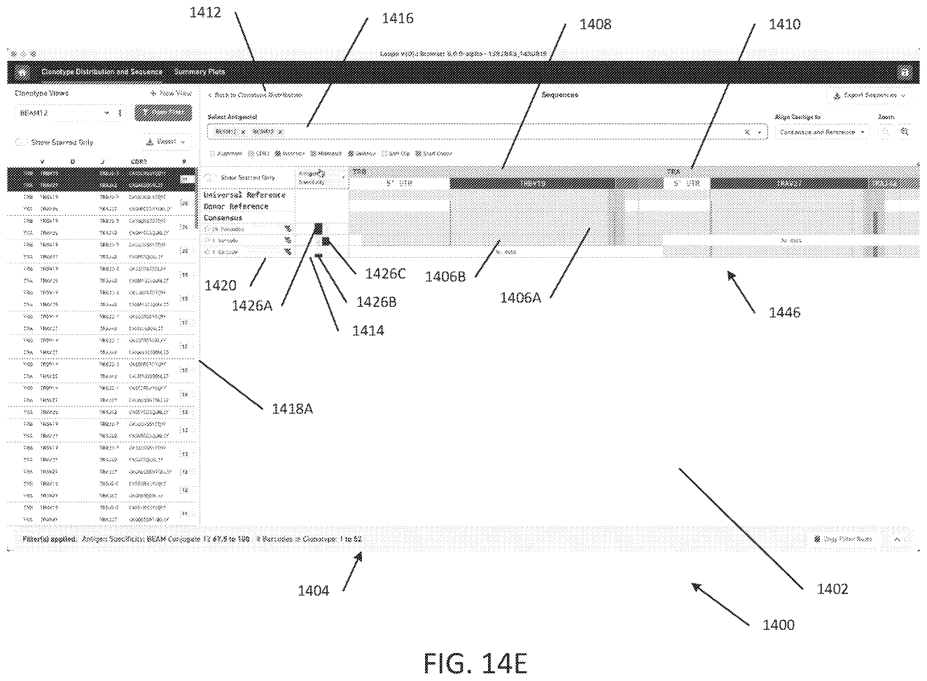

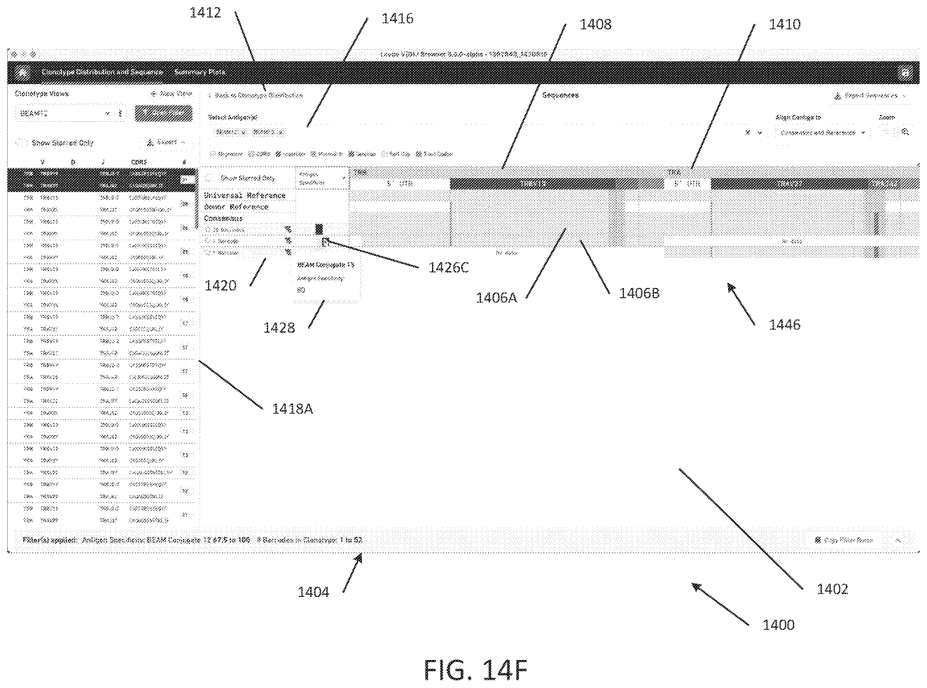

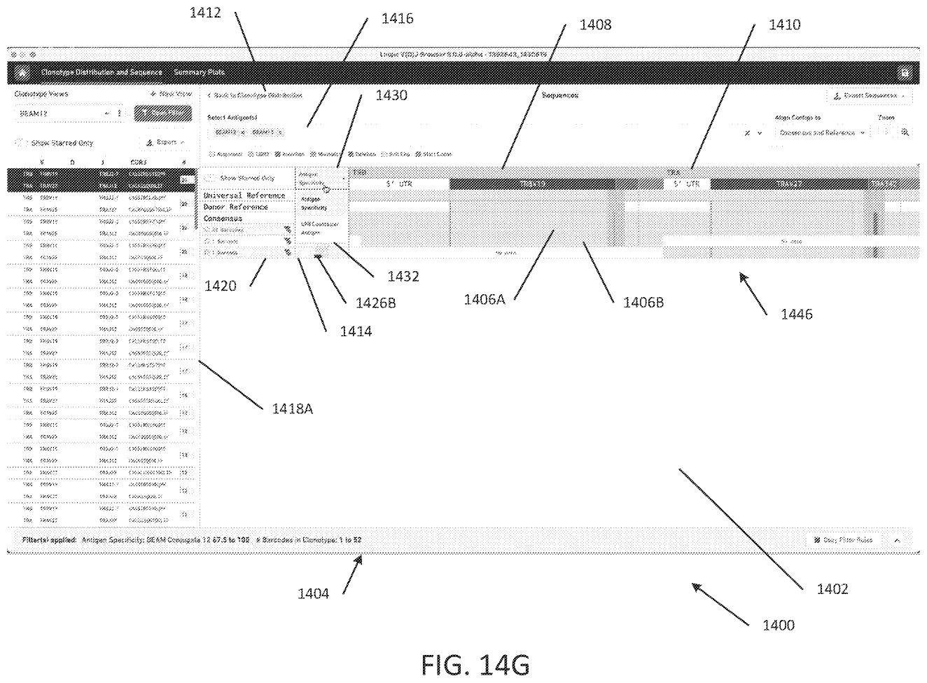

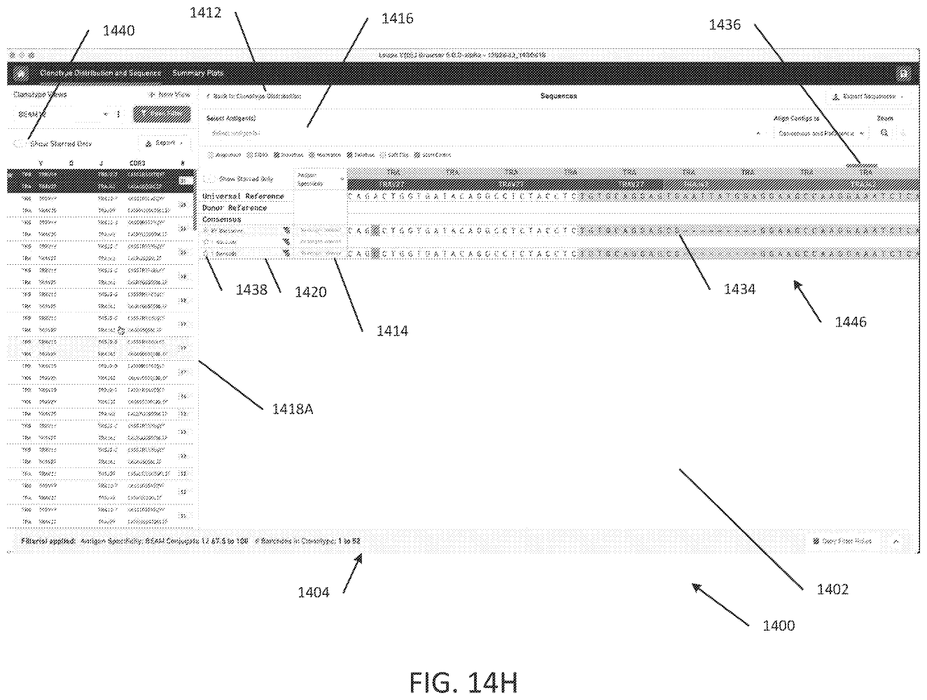

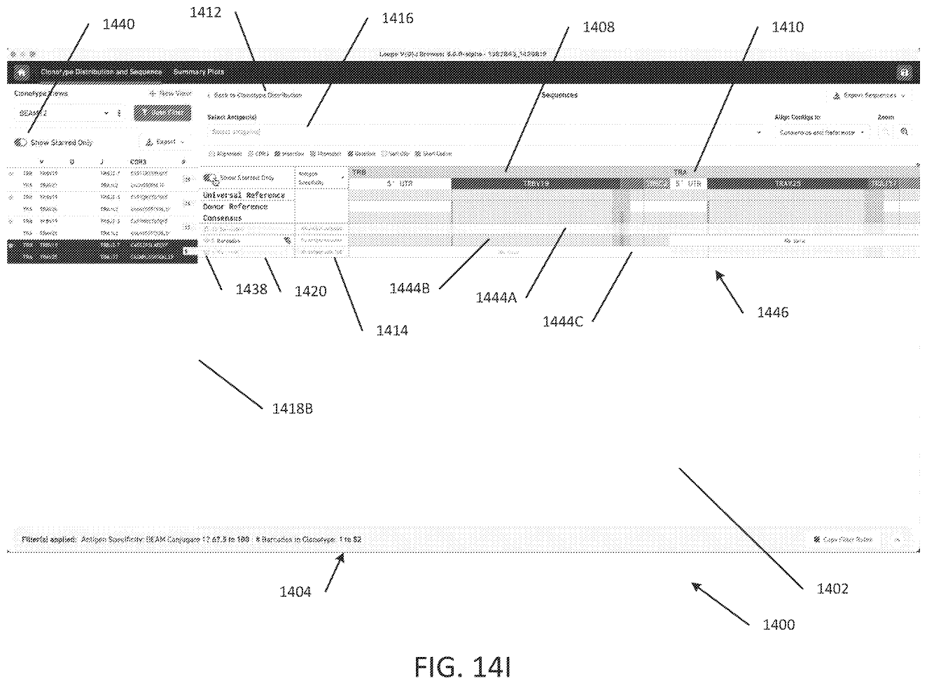

The following FIGS. 14A to 14I will illustrate various example output displays (see 1402 of FIG. 14A) of a visualization tool (see 1400 of FIG. 14A) for set of cellular data, illustrating the various features of the tool along with the real time, dynamic modifications to the outputs per user interactions (e.g., filtering). Though many of the features of previously discussed output displays will be included in these figures, discussion will largely be limited to those features changing from figure to figure.

FIG. 14A illustrates an example output display 1402 of a visualization tool 1400 for cellular data. Display 1402 includes a filter panel 1404. In the depicted views of example display 1402 filter panel 1404 is collapsed, though the filter panel 1404 may be expanded for use. Further detail regarding filtering is provided in the Dynamic Filtering section below. Display 1402 further includes a sequence table 1446. The sequence table 1446 includes one or more visual sequences corresponding to paired chains of respective cell receptors. For instance, in the depicted example, the sequence table 1446 includes a first visual sequence 1406A corresponding to a first chain 1408 (i.e. a label indicating the first chain) of a cell receptor and a second chain 1410 (i.e. a label indicating the second chain) of the cell receptor. For example, if the cell receptor is a B cell receptor, the first chain 1408 is a heavy chain and the second chain 1410 is a light chain, or vice versa. In another example, if the cell receptor is a T cell receptor, the first chain 1408 is an alpha chain and the second chain 1410 is a beta chain, or vice versa. The depicted example also shows a second visual sequence 1406B corresponding to a second cell receptor, and a third visual sequence without a reference numeral corresponding to a third cell receptor. Each visual sequence is in its own row of the sequence table 1446. Each of the rows are parallel to one another in the depicted example.

The sequence table 1446 further includes a barcode listing 1420 that lists in each respective cell receptor row the quantity of barcodes that support the paired chains of each respective cell receptor. In the depicted example, the cell receptor associated with the first visual sequence 1406A has twenty-nine barcodes, and thus twenty-nine contigs, in a subclonotype that support the paired first and second chains corresponding to the first visual sequence 1406A. The cell receptor associated with the second visual sequence 1406B has one barcode, and thus one contig, in a subclonotype that supports the paired first and second chains corresponding to the second visual sequence 1406B.

The sequence table 1446 may also include one or more visual sequences corresponding to a reference sequence and/or consensus sequence. In the depicted example, the sequence table 1446 includes a visual sequence in a row corresponding to a “Universal Reference” which is what all the rows of visual sequences below the “Universal Reference” are aligned to. The “Universal Reference” sequence is the published curated sequence of the genes identified in the selected paired chain (e.g., the first chain 1408 and second chain 1410). Additionally, the depicted example of the sequence table 1446 includes a visual sequence in a row corresponding to a “Donor Reference” which is the inferred germline sequence of the particular V, D, or J gene. The Donor Reference is included because the individual whose data is being visualized may have germline mutations in the individual's immune genes that would make the sequence different from the Universal Reference sequence. The depicted example of the sequence table 1446 further includes a visual sequence in a row corresponding to a chain consensus sequence (e.g., “Consensus”) from all the contigs that support the paired chains (e.g., the first chain 1408 and second chain 1410) of the selected clonotype. The chain consensus sequence always has an associated Universal Reference sequence, but may not always have an associated Donor Reference sequence.

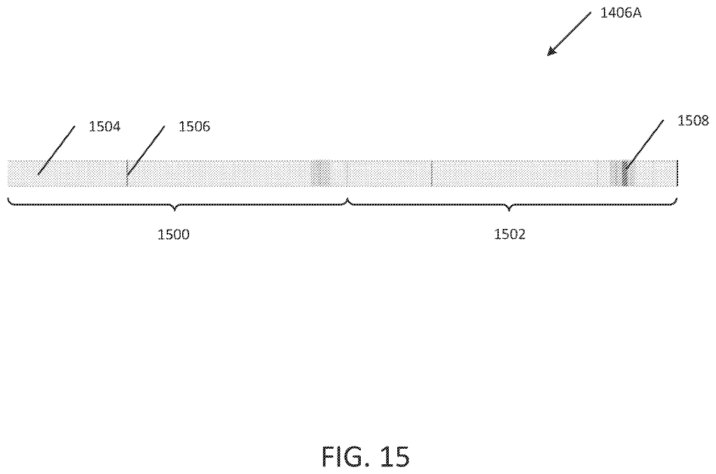

FIG. 15 illustrates the first visual sequence 1406A in more detail. The first visual sequence 1406A includes a first set of indicia 1500 that corresponds to first chain 1408 and a second set of indicia 1502 that corresponds to second chain 1410. Sets of indicia 1500 and 1502 visually represent the VDJ regions of the paired cell receptor chains (e.g., first chain 1408 and second chain 1410). For example, each of the one or more indicia in sets of indicia 1500 and 1502 may be a bar included in the row of the first visual sequence 1406A. Each of the one or more indicia are representative of information regarding the first visual sequence 1406A. For example, an indicia may be representative of one of: an area of the respective contig supporting a chain that covers the chain consensus sequence, complementarity determining region 3 (CDR3), an insertion incurred in the respective contig with respect to the chain consensus sequence, a mismatch between the respective contig and the chain consensus sequence, a deletion incurred in the respective contig with respect to the consensus sequence, soft-clipped sequence reads, a start codon of the respective contig, a stop codon of the respective contig, and a coding region of the respective contig. All of the information needed for generating the one or more indicia is included in the data set.

Each indicia representative of a certain type of information is distinguished from other indicia representative of a different type of information. For example, indicia representing different information may be different colors, shading, patterns, etc. In the depicted embodiment, display 1402 shows the possibility for indicia representative of an area of the respective contig supporting a chain that covers the chain consensus sequence (“Alignment”), complementarity determining region 3 (“CDR3”), an insertion, a mismatch, a deletion, soft-clipped sequence reads, and a start codon, though a visual sequence need not include all of these possible indicia. For example, set of indicia 1500 includes indicia for Alignment (e.g., indicia 1504), CDR3, an insertion, and a start Codon (e.g., indicia 1506) whereas set of indicia 1502 includes indicia for Alignment, CDR3, a mismatch, a deletion (e.g., indicia 1508), and a start codon. In an example, Alignment indicia may be light gray, CDR3 indicia may be dark gray, an insertion indicia may be blue, a mismatch indicia may be orange, a deletion indicia may be purple, Soft Clip indicia may be yellow, and a start codon indicia may be green.

In some instances, not all the contigs have contig consensus sequences that support the entire chain consensus sequence. For instance, returning to FIG. 14A, the second visual sequence 1406B does not have a sequence for region 1424 and so this area lacks any indicia. For example, region 1424 is whited out. Alternatively, region 1424 may include an indicia representative of the lack of a sequence for the region, such as a white bar. While this is an example of 5′ absence, the contigs may also have 3′ absence in their contig consensus sequences, which, in such instances, are treated the same as 5′ absence. As such, there can be regions indicative of a lack of a sequence to the left or the right in the contig consensus sequence of each contig. In some instances, the visual sequence of the chain consensus sequence may include indicia or lack of indicia indicative of a lack of a sequence, which means that that there were no reads assembled for that area in any of the contig consensus sequences that are aligned to form the chain consensus sequence.

Additionally, in some instances, the data set might not include information for parts of a cell receptor. In those instances, a visual sequence may display an indication of “No data” such as that seen in the depicted embodiment.

In at least some embodiments, the sequence table 1446 may include information on antigen specificity and/or UMI counts associated with the cell receptors depicted by the visual sequences in the sequence table 1446. For instance, the illustrated embodiment shows the sequence table 1446 having an antigen specificity/UMI count listing 1414. A user has not selected an antigen with the pull-down list 1416 (shown in a collapsed stated) in the depicted display 1402 of FIG. 14A, however, so the antigen specificity/UMI count listing 1414 does not include any information.

In various embodiments, display 1402 may include a table 1418A of clonotype information. Each row in table 1418A refers to the VDJ region of a cell receptor chain. The paired chains are grouped together in table 1418A such that the paired chains are selectable as a pair. A user may select any of the paired chains listed in table 1418A (e.g. by using a mouse to click on the rows representing the paired chains). When a new paired chains is selected, the sequence table 1446 is replaced with a new sequence table for the newly selected paired chains.

Display 1402 may include a button 1412, in various embodiments, that enables a user to return to the clonotype distribution plot. When a user selects button 1412 (e.g. by using a mouse to click on button 1412), table 1418A remains the same and the sequence table 1446 is replaced by a clonotype distribution plot.

FIG. 14B illustrates display 1402 when a user hovers over a label (e.g., “29 Barcodes”) in barcode listing 1420 in order to obtain a table 1422 of additional information on a subclonotype. For instance, in the depicted example, the additional information includes a total UMI count, a total read count, a full sequence, CDR3 AA, and CDR3 NT for each of first chain 1408 and second chain 1410. Table 1422 in this embodiment also includes a button for a user to select in order to download a list of barcodes for the subclonotype, which the user can import into another application (e.g., to explore gene expression differences in the cells expressing receptors of interest compared to the rest of the cells).

An advantage of the provided systems is that they enable viewing information regarding an antigen in conjunction with the sequence table 1446. FIG. 14C illustrates display 1402 when a user selects an antigen from the pull-down list 1416. As shown, the pull-down list 1416 is expanded and the user is selecting the antigen “BEAM12”.

FIG. 14D illustrates display 1402 showing representations of antigen specificity scores for the cell receptors in the sequence table 1446 after the antigen “BEAM12” is selected. For example, a histogram 1426A is displayed in the antigen specificity/UMI count listing 1414 for the cell receptor having twenty-nine barcodes. The histogram 1426A is a bar that fills the row of the cell receptor proportionate to the antigen specificity score it is representing. For instance, in the depicted example, the histogram 1426A fills the row entirely and therefore is proportionate to a maximum antigen specificity score (e.g., 100). Conversely, the histogram 1426B shown for the cell receptor having one barcode at the bottom of the sequence table 1446 (“middle cell receptor”) only fills a portion of the row. Therefore, the histogram 1426B represents an antigen specificity score less than the maximum (e.g., 20). The cell receptor having one barcode that does not have a histogram has a minimum antigen specificity score (e.g., 0).

In at least some embodiments, a user may select more than one antigen. FIG. 14E illustrates display 1402 after an additional antigen “BEAM13” is selected. As shown, display 1402 includes an additional column in the antigen specificity/UMI count listing 1414 for the representations of antigen specificity scores for the cell receptors with respect to “BEAM13”. In this example, only the middle cell receptor having a histogram 1426C has an antigen specificity score greater than the minimum with respect to “BEAM13”.

An advantage of some embodiments is that the provided systems enable a user to overlay antigen specific information on display 1402 which can be helpful for users to put the paired chain sequences in context of the binding properties of antigens. FIG. 14F illustrates display 1402 showing an overlay 1428 generated and displayed for the middle cell receptor. In at least some examples, overlay 1428 is displayed in response to a user interaction with display 1402, such as a user hovering a cursor over a histogram in antigen specificity/UMI count listing 1414. In the depicted example, a cursor is hovered over histogram 1426C so that overlay 1428 is displayed. Overlay 1428 may include suitable information regarding antigen specificity for a selected antigen. For example, in the depicted embodiment, overlay 1428 shows an antigen name (“BEAM Conjugate 13”) and an antigen specificity score of 80.

In addition to antigen specificity scores, in various embodiments, the provided systems also enable a user to overlay UMI count information. FIG. 14G illustrates display 1402 showing a user having pressed a button 1430 to expand a drop-down list 1432. The drop-down list 1432 indicates that a user may select between antigen specificity and UMI counts per antigen. If the user selects UMI counts per antigen, the histograms in antigen specificity/UMI count listing 1414 will update proportionately to the UMI counts for each cell receptor and antigen, and overlay 1428 will update to display UMI counts instead of antigen specificity scores. Further information regarding how the antigen specificity score and the UMI counts are determined is provided below in the Antigen Specificity and UMI Count/Antigen section.

In at least some embodiments, the provided systems enable a user to view the full sequences of the cell receptors as opposed to only the visual sequences discussed above. For example, FIG. 14H illustrates display 1402 showing a portion of a full sequence 1434 associated with the cell receptor having twenty-nine barcodes as well as a full sequence for the bottom cell receptor having one barcode. To get to the full sequence view, in one example, a user may click on the sequence table 1446. Because full sequence 1434 is too long to fit on display 1402 at once, the user may use a scroll bar 1436 to scroll through various portions of full sequence 1434 that interest the user.

Also shown in the various depictions of display 1402 are selectable indicators 1438. Each of the cell receptors in the sequence table 1446 includes a selectable indicator 1438. A user may select a selectable indicator 1438 for a cell receptor that the user finds particularly interesting or relevant so that the user can quickly reference that cell receptor in the future. For instance, a user may review the sequence table 1446 and antigen information and find a particular cell receptor to be potentially useful for a future experiment, and then review the full sequences and find another cell receptor to be potentially useful. Enabling researchers (e.g., immunology researchers) to view such a vast amount of information in a single place in order to identify relevant cell receptors and clonotypes can be very helpful in advancing immunology research and lead to discoveries in the field. When the user selects a selectable indicator 1438, the selectable indicator 1438 may change color, shape, shading, etc. to distinguish a selected selectable indicator 1438 and an unselected selectable indicator 1438. For example, the selectable indicator 1438 for the cell receptor having twenty-nine barcodes is selected and a filled in star whereas the selectable indicators 1438 for the two other cell receptors are not selected and are merely star outlines. While the selectable indicators 1438 are depicted as stars in the illustrated embodiment, they may have any suitable shape in other examples.

Display 1402 may also provide an option for the user to view only the clonotypes including cell receptors that the user found particularly interesting or relevant. For example, display 1402 may include a toggle button 1440 that a user may activate to modify the table 1418A so that only clonotypes are listed that include a cell receptor with a selected selectable indicator 1438. Additionally, it can be noted that, in the display 1402 of FIG. 14H, the user has removed all of the selected antigens so that antigen specificity/UMI count listing 1414 is empty again.

FIG. 14I illustrates display 1402 after the user has activated the toggle button 1440. As shown, only clonotypes having a star next to them are displayed in a modified table 1418B. In this way, a user can view all of the clonotypes and cell receptors that the user finds most relevant to the user's research rather than having to click back through the display 1402 many times to find ones the user wants to reference again, which can help to increase research efficiency. The provided systems additionally enable a user to export data associated solely with the starred clonotypes and cell receptors. For instance, the provided systems generate an export file including such information in response to a user selecting an export button with the toggle button 1440 activated.

In accordance with various embodiments, the sequence table 1446 may be modified in response to the user activating the toggle button 1440. For instance, after toggle button 1440 is activated, visual sequences corresponding to cell receptors that have unselected selectable indicators 1438 may be modified. For example, visual sequences corresponding to cell receptors that have unselected selectable indicators 1438 may be removed or grayed out. It can be noted that, in the display 1402 of FIG. 14I, the user has clicked a new clonotype in the modified table 1418B such that a sequence table 1446 is displayed for that clonotype. In the depicted example, the visual sequence 1444B is starred and remains unmodified. Conversely, the visual sequences 1444A and 1444C are unselected and are grayed out. Graying out unselected visual sequences rather than removing them entirely enables users to still see the unselected visual sequences and potentially gather information from them.

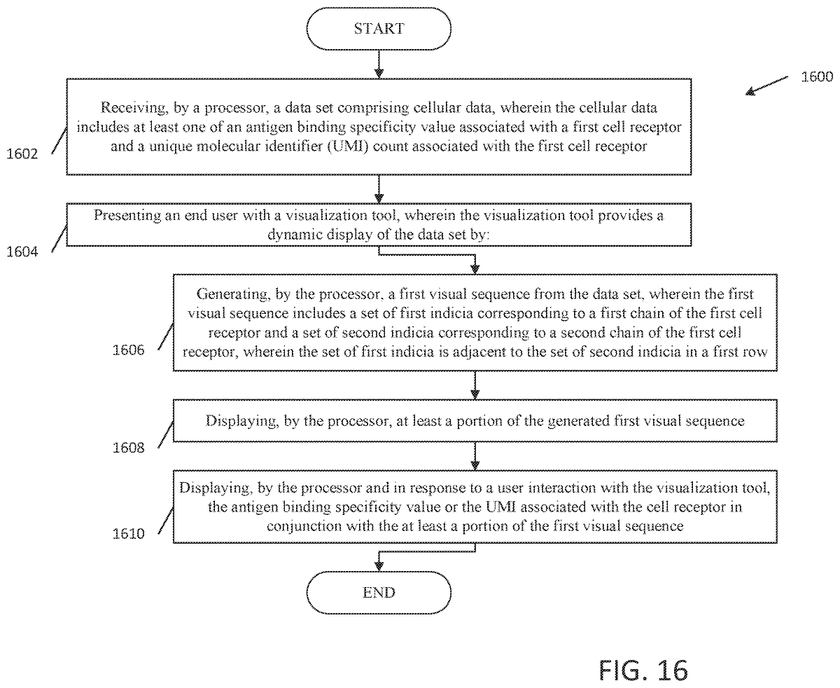

In accordance with various embodiments, a computer-implemented method for a visualization tool for cellular data is provided. The method can comprise a series of steps or operations illustrated in FIG. 16 and may be performed by processing logic that may comprise hardware (circuitry, dedicated logic, etc.), software, or a combination of both. For example, the method can comprise receiving, by a processor, a data set comprising cellular data, wherein the cellular data includes at least one of an antigen binding specificity value associated with a first cell receptor and a unique molecular identifier (UMI) count associated with the first cell receptor (see, e.g., operation 1602). The method can further comprise presenting an end user with a visualization tool (see, e.g., operation 1604). The visualization tool can provide a dynamic display of the data set by generating, by the processor, a first visual sequence from the data set (see, e.g., operation 1604), displaying, by the processor, at least a portion of the generated first visual sequence (see, e.g., operation 1608), and displaying, by the processor and in response to a user interaction with the visualization tool, the antigen binding specificity value or the UMI associated with the cell receptor in conjunction with the at least a portion of the first visual sequence (see, e.g., operation 1610). The first visual sequence includes a set of first indicia corresponding to a first chain of the first cell receptor and a set of second indicia corresponding to a second chain of the first cell receptor, wherein the set of first indicia is adjacent to the set of second indicia in a first row. The dynamic display of the data set can provide for analysis of the cellular data from the data set. Further detailed discussion regarding the various features of the dynamic display will be provided below.

Further, in accordance with various embodiments for a computer implemented method of a visualization tool for cellular data, the method can further comprise generating, by the processor, a second visual sequence from the data set, wherein the second visual sequence includes a set of third indicia corresponding to a first chain of a second cell receptor and a set of fourth indicia corresponding to a second chain of the second cell receptor, wherein the set of third indicia is adjacent to the set of fourth indicia in a second row; and displaying, by the processor, at least a portion of the generated second visual sequence. The first and second rows are parallel.

In addition, in accordance with various embodiments for a computer implemented method of a visualization tool for cellular data, the data set is an output of a process that consolidates a plurality of discrete data sets such that the data set is in a format that enables the processor to display the antigen binding specificity value or the UMI associated with the cell receptor in conjunction with one of the first and second portions of the first visual sequence.

Further, in accordance with various embodiments for a computer implemented method of a visualization tool for cellular data, the data set is contained in a multi-section data file executed by the processor, wherein a first section of the data file includes a concatenated string of raw sequences and alignment information for clonotype chains, exact clonotype chains, and donor and universal references.

Additionally, in accordance with various embodiments for a computer implemented method of a visualization tool for cellular data, the method can further comprise generating, by the processor, a plurality of visual sequences including the first visual sequence and displaying, by the processor, at least a portion of each visual sequence in its own discrete row; generating, by the processor, a table of information from the data set and displaying, by the processor, the table of information, wherein the table of information includes genetic information for at least one of identified clonotypes and cell receptors belonging to a clonotype that are presented in the plurality of visual sequences; receiving a first user selection of the first visual sequence; receiving a second user selection of a third visual sequence of the plurality of visual sequences; and modifying, by the processor, the table of information to generate a modified table of information. The information on the plurality of visual sequences is removed from the modified table of information except for the information on the selected first and third visual sequences.

As well, in accordance with various embodiments, the method can further comprise generating, by the processor and in response to a second user interaction with the visualization tool, a file including the information on the selected first and third visual sequences.

Further, in accordance with various embodiments for a computer implemented method of a visualization tool for cellular data, the antigen binding specificity value is determined based on a cumulative distribution function of the beta distribution associated with the cell receptor binding to a target antigen and the cell receptor binding to a control antigen.

Moreover, in accordance with various embodiments of a computer implemented method for a visualization tool for cellular data, the UMI count includes a quantity of UMIs associated with a target antigen bound to the cell receptor.

Further, in accordance with various embodiments of a computer implemented method for a visualization tool for cellular data, the cellular data includes T-Cell data, and the first chain is an alpha chain and the second chain is a beta chain.

Further, in accordance with various embodiments of a computer implemented method for a visualization tool for cellular data, the cellular data includes B-Cell data, and wherein the first chain is a heavy chain and the second chain is a light chain.

Further, in accordance with various embodiments of a computer implemented method for a visualization tool for cellular data, the method can further comprise receiving, by the processor, a filter selection, wherein the filter selection is selected from the group consisting of: UMI Counts/antigen, antigen specificity, barcode, number of barcodes representing a clonotype, CDR3 amino acid sequence, CDR3 bases, gene name, isotype, cluster, amino acid % mutation, nucleotide % mutation, iNKT/MAIT, and combinations thereof, modifying, by the processor, the first visual sequence based on the filter selection, to generate a modified first visual sequence that is different from the first visual sequence, and displaying the modified first visual sequence.

Further, in accordance with various embodiments of a computer implemented method for a visualization tool for cellular data, the method can further comprise generating, by the processor, a first table of information from the data set and displaying, by the processor, the first table; and modifying, by the processor, the first table based on the filter selection, to generate a modified first table of information that is different from the first table, and displaying, by the processor, the modified first table, wherein at least one of the first table and modified first table comprises genetic information for identified clonotypes or cell receptors belonging to a clonotype presented in the at least one of the first visual sequence and the modified first visual sequence.

Further, in accordance with various embodiments of a computer implemented method for a visualization tool for cellular data, the genetic information is selected from the group consisting or V-gene, D-gene, J-gene, CDR sequence, and combinations thereof.

Further, in accordance with various embodiments of a computer implemented method for a visualization tool for cellular data, the filter is selected from multiple properties of VDJ sequences for heavy and light chains.

Further, in accordance with various embodiments of a computer implemented method for a visualization tool for cellular data, the at least a portion of the first visual sequence includes the set of first indicia corresponding to the first chain of the first cell receptor and the set of second indicia corresponding to the second chain of the first cell receptor.

Further, in accordance with various embodiments of a computer implemented method for a visualization tool for cellular data, displaying the at least a portion of the first visual sequence includes: displaying, by the processor, a first portion of the generated first visual sequence, and displaying, by the processor, a second portion of the generated first visual sequence in response to a first user interaction with the visualization tool.

Further, in accordance with various embodiments for a computer implemented method of a visualization tool for cellular data, the method can further comprise receiving a plurality of discrete data sets from one or more data sources, and generating a multi-section data file that combines the plurality of discrete data sets. In such embodiments, the multi-section data file is the data set received at operation 1602. The plurality of discrete data sets may be received by, and the multi-section data file may be generated by, a processor (e.g., a first processor) different than the processor (e.g., a second processor) that performs operations 1602-1610.

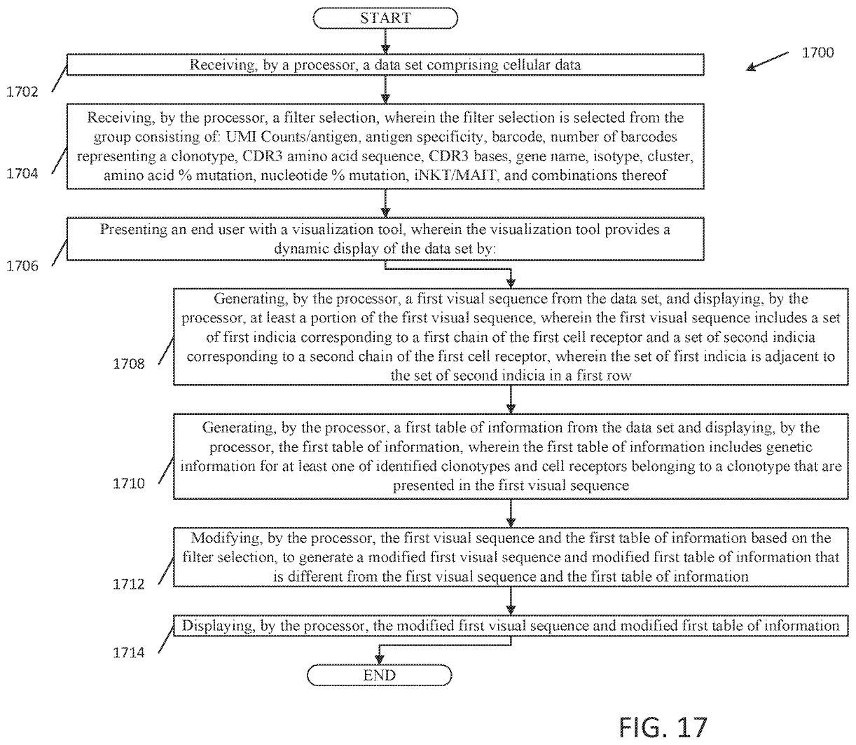

In accordance with various embodiments, a computer-implemented method for a visualization tool for cellular data is provided. The method can comprise a series of steps or operations illustrated in FIG. 17 and may be performed by processing logic that may comprise hardware (circuitry, dedicated logic, etc.), software, or a combination of both. For example, the method can comprise receiving, by a processor, a data set comprising cellular data, (see, e.g., operation 1702). The method can further comprise receiving, by the processor, a filter selection, wherein the filter selection is selected from the group consisting of: UMI Counts/antigen, antigen specificity, barcode, number of barcodes representing a clonotype, CDR3 amino acid sequence, CDR3 bases, gene name, isotype, cluster, amino acid % mutation, nucleotide % mutation, iNKT/MAIT, and combinations thereof (see, e.g., operation 1704). The method can further comprise presenting an end user with a visualization tool (see, e.g., operation 1706). The visualization tool can provide a dynamic display of the data set by generating, by the processor, a first visual sequence from the data set and, displaying, by the processor, at least a portion of the generated first visual sequence (see, e.g., operation 1708), generating, by the processor, a first table of information from the data set and displaying, by the processor, the first table of information, (see, e.g., operation 1710), modifying, by the processor, the first visual sequence and the first table of information based on the filter selection, to generate a modified first visual sequence and modified first table of information that is different from the first visual sequence and the first table of information (see, e.g., operation 1712), and displaying, by the processor, the modified first visual sequence and modified first table of information (see, e.g., operation 1714). The first visual sequence includes a set of first indicia corresponding to a first chain of the first cell receptor and a set of second indicia corresponding to a second chain of the first cell receptor, wherein the set of first indicia is adjacent to the set of second indicia in a first row. The first table of information includes genetic information for at least one of identified clonotypes and cell receptors belonging to a clonotype that are presented in the first visual sequence. The dynamic display of the data set can provide for analysis of the cellular data from the data set. Further detailed discussion regarding the various features of the dynamic display will be provided below.

Further, in accordance with various embodiments for a computer implemented method of a visualization tool for cellular data, the method can further comprise generating, by the processor, a second visual sequence from the data set, wherein the second visual sequence includes a set of third indicia corresponding to a first chain of a second cell receptor and a set of fourth indicia corresponding to a second chain of the second cell receptor, wherein the set of third indicia is adjacent to the set of fourth indicia in a second row; and displaying, by the processor, at least a portion of the generated second visual sequence. The first and second rows are parallel.

In addition, in accordance with various embodiments for a computer implemented method of a visualization tool for cellular data, the data set is an output of a process that consolidates a plurality of discrete data sets such that the data set is in a format that enables the processor to display the antigen binding specificity value or the UMI associated with the cell receptor in conjunction with one of the first and second portions of the first visual sequence.

Further, in accordance with various embodiments for a computer implemented method of a visualization tool for cellular data, the data set is contained in a multi-section data file executed by the processor, wherein a first section of the data file includes a concatenated string of raw sequences and alignment information for clonotype chains, exact clonotype chains, and donor and universal references.

Additionally, in accordance with various embodiments for a computer implemented method of a visualization tool for cellular data, the method can further comprise generating, by the processor, a plurality of visual sequences including the first visual sequence and displaying, by the processor, at least a portion of each visual sequence in its own discrete row; generating, by the processor, a table of information from the data set and displaying, by the processor, the table of information, wherein the table of information includes genetic information for at least one of identified clonotypes and cell receptors belonging to a clonotype that are presented in the plurality of visual sequences; receiving a first user selection of the first visual sequence; receiving a second user selection of a third visual sequence of the plurality of visual sequences; and modifying, by the processor, the table of information to generate a modified table of information. The information on the plurality of visual sequences is removed from the modified table of information except for the information on the selected first and third visual sequences.

As well, in accordance with various embodiments, the method can further comprise generating, by the processor and in response to a second user interaction with the visualization tool, a file including the information on the selected first and third visual sequences.

Further, in accordance with various embodiments for a computer implemented method of a visualization tool for cellular data, the cellular data includes at least one of an antigen binding specificity value associated with a first cell receptor and a unique molecular identifier (UMI) count associated with the first cell receptor, and the method can further comprise displaying, by the processor and in response to a user interaction with the visualization tool, the antigen binding specificity value or the UMI associated with the cell receptor in conjunction with the at least a portion of the first visual sequence.

Further, in accordance with various embodiments for a computer implemented method of a visualization tool for cellular data, the antigen binding specificity value is determined based on a cumulative distribution function of the beta distribution associated with the cell receptor binding to a target antigen and the cell receptor binding to a control antigen.

Moreover, in accordance with various embodiments of a computer implemented method for a visualization tool for cellular data, the UMI count includes a quantity of UMIs associated with a target antigen bound to the cell receptor.

Further, in accordance with various embodiments of a computer implemented method for a visualization tool for cellular data, the cellular data includes T-Cell data, and the first chain is an alpha chain and the second chain is a beta chain.

Further, in accordance with various embodiments of a computer implemented method for a visualization tool for cellular data, the cellular data includes B-Cell data, and wherein the first chain is a heavy chain and the second chain is a light chain.

Further, in accordance with various embodiments of a computer implemented method for a visualization tool for cellular data, the genetic information is selected from the group consisting or V-gene, D-gene, J-gene, CDR sequence, and combinations thereof.

Further, in accordance with various embodiments of a computer implemented method for a visualization tool for cellular data, the filter is selected from multiple properties of VDJ sequences for heavy and light chains.

Further, in accordance with various embodiments of a computer implemented method for a visualization tool for cellular data, the at least a portion of the first visual sequence includes the set of first indicia corresponding to the first chain of the first cell receptor and the set of second indicia corresponding to the second chain of the first cell receptor.

Further, in accordance with various embodiments of a computer implemented method for a visualization tool for cellular data, displaying the at least a portion of the first visual sequence includes: displaying, by the processor, a first portion of the generated first visual sequence, and displaying, by the processor, a second portion of the generated first visual sequence in response to a first user interaction with the visualization tool.

Further, in accordance with various embodiments for a computer implemented method of a visualization tool for cellular data, the method can further comprise receiving a plurality of discrete data sets from one or more data sources, and generating a multi-section data file that combines the plurality of discrete data sets. In such embodiments, the multi-section data file is the data set received at operation 1602.

In accordance with various embodiments, a system for a visualization tool for cellular data is disclosed. The system includes a memory and a processor in communication with the memory. The processor is configured to perform the operations comprising receiving a data set comprising cellular data, wherein the cellular data includes at least one of an antigen binding specificity value associated with a first cell receptor and a unique molecular identifier (UMI) count associated with the first cell receptor. The operations can further comprise presenting an end user with a visualization tool. The visualization tool can provide a dynamic display of the data set via additional operations that the processor is configured to perform comprising generating a first visual sequence from the data set, displaying, at least a portion of the generated first visual sequence, and displaying, in response to a user interaction with the visualization tool, the antigen binding specificity value or the UMI associated with the cell receptor in conjunction with the at least a portion of the first visual sequence. The first visual sequence includes a set of first indicia corresponding to a first chain of the first cell receptor and a set of second indicia corresponding to a second chain of the first cell receptor, wherein the set of first indicia is adjacent to the set of second indicia in a first row. The dynamic display of the data set can provide for analysis of the cellular data from the data set.

In accordance with various embodiments, a system for a visualization tool for cellular data is disclosed. The system includes a memory and a processor in communication with the memory. The processor is configured to perform the operations comprising receiving, a data set comprising cellular data. The operations can further comprise receiving, a filter selection, wherein the filter selection is selected from the group consisting of: UMI Counts/antigen, antigen specificity, barcode, number of barcodes representing a clonotype, CDR3 amino acid sequence, CDR3 bases, gene name, isotype, cluster, amino acid % mutation, nucleotide % mutation, iNKT/MAIT, and combinations thereof. The operations can further comprise presenting an end user with a visualization tool. The visualization tool can provide a dynamic display of the data set via additional operations that the processor is configured to perform comprising generating, a first visual sequence from the data set and, displaying, at least a portion of the generated first visual sequence, generating, a first table of information from the data set and displaying, the first table of information, modifying, the first visual sequence and the first table of information based on the filter selection, to generate a modified first visual sequence and modified first table of information that is different from the first visual sequence and the first table of information, and displaying, the modified first visual sequence and modified first table of information. The first visual sequence includes a set of first indicia corresponding to a first chain of the first cell receptor and a set of second indicia corresponding to a second chain of the first cell receptor, wherein the set of first indicia is adjacent to the set of second indicia in a first row. The first table of information includes genetic information for at least one of identified clonotypes and cell receptors belonging to a clonotype that are presented in the first visual sequence. The dynamic display of the data set can provide for analysis of the cellular data from the data set.

In accordance with various embodiments, a system for a visualization tool for cellular data is disclosed. The system includes a first memory and a first processor in communication with the first memory, and a second memory and a second processor in communication with the second memory. The first processor is configured to perform first operations comprising receiving a plurality of discrete data sets from one or more data sources. The first operations further comprise generating a multi-section data file that combines the plurality of discrete data sets, wherein the multi-section data file includes cellular data including at least one of an antigen binding specificity value associated with a first cell receptor and a unique molecular identifier (UMI) associated with the first cell receptor. The second processor is configured to perform second operations comprising receiving the multi-section data file, and presenting an end user with a visualization tool. The visualization tool can provide a dynamic display of the multi-section data file via additional second operations that the second processor is configured to perform comprising generating a first visual sequence from the multi-section data file and, displaying at least a portion of the generated first visual sequence, generating a first table of information from the multi-section data file and displaying the first table of information, modifying the first visual sequence and the first table of information based on the filter selection, to generate a modified first visual sequence and modified first table of information that is different from the first visual sequence and the first table of information, and displaying the modified first visual sequence and modified first table of information. The first visual sequence includes a set of first indicia corresponding to a first chain of the first cell receptor and a set of second indicia corresponding to a second chain of the first cell receptor, wherein the set of first indicia is adjacent to the set of second indicia in a first row. The first table of information includes genetic information for at least one of identified clonotypes and cell receptors belonging to a clonotype that are presented in the first visual sequence. The dynamic display of the multi-section data file can provide for analysis of the cellular data from the multi-section data file.

In accordance with various embodiments, a system for a visualization tool for cellular data is disclosed. The system includes a first memory and a first processor in communication with the first memory, and a second memory and a second processor in communication with the second memory. The first processor is configured to perform first operations comprising receiving a plurality of discrete data sets from one or more data sources, and generating a multi-section data file that combines the plurality of discrete data sets, wherein the multi-section data file includes cellular data including at least one of an antigen binding specificity value associated with a first cell receptor and a unique molecular identifier (UMI) associated with the first cell receptor. The second processor is configured to perform second operations comprising receiving the multi-section data file, and presenting an end user with a visualization tool. The visualization tool can provide a dynamic display of the multi-section data file via additional second operations that the second processor is configured to perform comprising generating a first visual sequence from the multi-section data file and, displaying at least a portion of the generated first visual sequence, generating a first table of information from the multi-section data file and displaying the first table of information, modifying the first visual sequence and the first table of information based on the filter selection, to generate a modified first visual sequence and modified first table of information that is different from the first visual sequence and the first table of information, and displaying the modified first visual sequence and modified first table of information. The first visual sequence includes a set of first indicia corresponding to a first chain of the first cell receptor and a set of second indicia corresponding to a second chain of the first cell receptor, wherein the set of first indicia is adjacent to the set of second indicia in a first row. The first table of information includes genetic information for at least one of identified clonotypes and cell receptors belonging to a clonotype that are presented in the first visual sequence. The dynamic display of the multi-section data file can provide for analysis of the cellular data from the multi-section data file.

In accordance with various embodiments, a non-transitory, computer-readable medium storing instructions is provided. The instructions, when executed by a processor, cause the processor to perform operations comprising receiving a data set comprising cellular data, wherein the cellular data includes at least one of an antigen binding specificity value associated with a first cell receptor and a unique molecular identifier (UMI) count associated with the first cell receptor. The operations can further comprise presenting an end user with a visualization tool. The visualization tool can provide a dynamic display of the data set via additional operations that the processor is configured to perform comprising generating a first visual sequence from the data set, displaying, at least a portion of the generated first visual sequence, and displaying, in response to a user interaction with the visualization tool, the antigen binding specificity value or the UMI associated with the cell receptor in conjunction with the at least a portion of the first visual sequence. The first visual sequence includes a set of first indicia corresponding to a first chain of the first cell receptor and a set of second indicia corresponding to a second chain of the first cell receptor, wherein the set of first indicia is adjacent to the set of second indicia in a first row. The dynamic display of the data set can provide for analysis of the cellular data from the data set.

In accordance with various embodiments, a non-transitory, computer-readable medium storing instructions is provided. The instructions, when executed by a processor, cause the processor to perform operations comprising receiving, a data set comprising cellular data. The operations can further comprise receiving, a filter selection, wherein the filter selection is selected from the group consisting of: UMI Counts/antigen, antigen specificity, barcode, number of barcodes representing a clonotype, CDR3 amino acid sequence, CDR3 bases, gene name, isotype, cluster, amino acid % mutation, nucleotide % mutation, iNKT/MAIT, and combinations thereof. The operations can further comprise presenting an end user with a visualization tool. The visualization tool can provide a dynamic display of the data set via additional operations that the processor is configured to perform comprising generating, a first visual sequence from the data set and, displaying, at least a portion of the generated first visual sequence, generating, a first table of information from the data set and displaying, the first table of information, modifying, the first visual sequence and the first table of information based on the filter selection, to generate a modified first visual sequence and modified first table of information that is different from the first visual sequence and the first table of information, and displaying, the modified first visual sequence and modified first table of information. The first visual sequence includes a set of first indicia corresponding to a first chain of the first cell receptor and a set of second indicia corresponding to a second chain of the first cell receptor, wherein the set of first indicia is adjacent to the set of second indicia in a first row. The first table of information includes genetic information for at least one of identified clonotypes and cell receptors belonging to a clonotype that are presented in the first visual sequence. The dynamic display of the data set can provide for analysis of the cellular data from the data set.

For more detail regarding customization of visualizations, in accordance with various embodiments, refer to the Additional Features of Clonotype Data Visualization section below for detailed discussion. It should be noted that the various parameters, variables, fields, values, filters, etc. discussed in detail herein are independent and interchangeable in any contemplated fashion or combination. Moreover, the various parameters, variables, fields, values, filters, etc. discussed in detail herein are applicable to any and all the various embodiments discussed or contemplated herein.

### Antigen Specificity and UMI Count/Antigen

As described above, in some embodiments, the sequence data associated with the immune cell includes a UMI Count/antigen and/or an antigen specificity determination. The antigen specificity of an antigen binding molecule (ABM), such as the antigen receptor of an immune cell (e.g., a B lymphocyte, a T lymphocyte, and/or the like), may be largely determined by the complementarity determining regions (CDRs) of the receptor expressed by the immune cell. Identifying antibody binding molecules that binds to a target antigen with high affinity and that neutralizes the antigen may be crucial for disease prevention and treatment. Nevertheless, identifying antigen binding molecules with sufficient binding specificity towards a target antigen may be time consuming and resource intensive endeavor, particularly when antigen binding specificity is being assessed at a single cell resolution.

As such, various aspects of the present disclosure include techniques for assessing antigen binding specificity to support high throughput discovery of immune cells capable of binding to and neutralizing various target antigens. For example, in some example embodiments, the antigen binding specificity of an antigen binding molecule, such as an antigen receptor expressed by an immune cell, may be assessed by determining a specificity metric for the antigen binding molecule. The specificity metric of the antigen binding molecule may correspond to a likelihood of the antigen binding molecule binding to the target antigen at above a certain threshold. For instance, in some cases, the specificity metric of the antigen binding molecule may be determined based on a first measurement of the target antigen bound to the antigen binding molecule and a second measurement of a control antigen bound to the antigen binding molecule. Whether the antigen binding molecule exhibit sufficient specificity towards the target antigen may be determined based on the specificity metric of the antigen binding molecule.

As described in more detail below, one aspect of the disclosure relates to new methods and systems for assessing the antigen specificity of an antigen binding molecule (ABM) expressed by one or more cells. For example, in some example embodiments, assessing the antigen specificity of the antigen binding molecule may include determining whether the antigen binding molecule is capable of binding to a target antigen with sufficient specificity. Examples of the antigen binding molecule include immune cell receptors such as antibodies (Abs), antigen-binding fragments of antibodies, B cell receptors (BCR), antigen-binding fragments of B cell receptors, T cell receptors (TCR), and antigen-binding fragments of T cell receptors. The target antigen may be any target antigen of interest. Examples of target antigens may include a spike(S) protein of a coronavirus (CoV-S), e.g., a severe acute respiratory syndrome coronavirus 1 (SARS-CoV-1), a SARS-CoV-2, a Middle East respiratory syndrome coronavirus (MERS-CoV), and/or the like. Other examples of target antigens include an immune checkpoint molecule (e.g., CD38, PD-1, CTLA-4, TIGIT, LAG-3, VISTA, TIM-3), an influenza hemagglutinin, a human immunodeficiency virus (HIV) envelope protein, a cytokine, a viral glycoprotein, and/or the like.

In some example embodiments, the antigen specificity of an antigen binding molecule may be determined based on a specificity metric that corresponds to a likelihood of the antigen binding molecule binding to the target antigen at above a certain threshold. For example, as noted, the specificity metric of the antigen binding molecule may be determined based on a first measurement of the target antigen bound to the antigen binding molecule and a second measurement of a control antigen bound to the antigen binding molecule. A higher specificity metric, such as a specificity metric that exceeds a threshold value may indicate that the antigen binding molecule is capable of binding to the target antigen with a high specificity.

Referring again to FIG. 1, in some example embodiments, the data source 110 can be configured to obtain a data set that includes, for instance, data associated with reporter oligonucleotides that are operatively coupled (e.g., directly or indirectly conjugated) to antigens. As noted, a reporter oligonucleotide operatively coupled to an antigen may include a sequence, such as a reporter barcode sequence, that enables an identification of the antigen. Conjugating antigens with oligonucleotides that include unique reporter barcode sequences may be useful for differentiation between different antigens, for example, during a multiplexed antigen assay. To further facilitate the processing and identification of the reporter barcode sequence (e.g., through nucleic acid sequencing), the reporter oligonucleotide may also include additional sequences including, for example, an adapter sequence, a primer sequence, a primer binding sequence, and/or a unique molecular identifier (UMI).

In some example embodiments, the data set received from the data source 110 may include a first measurement of a target antigen bound to an antigen binding molecule expressed by one or more cells. The first measurement may correspond to a quantity of unique molecular identifiers (UMIs) associated with the target antigen bound to the antigen binding molecule. In some cases, the first measurement may be a sum of the quantities of unique molecular identifiers (UMIs) associated with multiple related target antigens. For example, the first measurement may correspond to a sum of a first quantity of unique molecular identifiers (UMIs) associated with a first target antigen bound to the antigen binding molecule and a second quantity of unique molecular identifiers (UMIs) associated with a second target antigen bound to the antigen binding molecule.

In some example embodiments, the data set received from the data source 110 may include a second measurement of a control antigen bound to the antigen binding molecule expressed by one or more cells. The second measurement may correspond to a quantity of unique molecular identifiers (UMIs) associated with the control antigen bound to the antigen binding molecule. In some cases where there are multiple related control antigens, the second measurement may be a sum of the quantities of unique molecular identifiers (UMIs) associated with at least one (e.g., each) control antigen. For instance, the second measurement may correspond to a sum of a first quantity of unique molecular identifiers (UMIs) associated with a first control antigen bound to the antigen binding molecule and a second quantity of unique molecular identifiers (UMIs) associated with a second control antigen bound to the antigen binding molecule.

The processing unit 140, or a separate processing unit in communication with the processing unit 140, may determine, based at least on the data set received form the data source 110, a specificity metric sigₚ for the antigen binding molecule (ABM). For example, the processing unit 140 may determine, based least on the first measurement of the target antigen bound to the antigen binding molecule and the second measurement of the control antigen bound to the antigen binding molecule, the specificity metric sigₚ for the antigen binding molecule. In some cases, the specificity metric sign for the antigen binding molecule may be determined based on a cumulative distribution function (CDF) of the beta distribution associated with the antigen binding molecule binding to the target antigen and the antigen binding molecule binding to the control antigen. This computation is shown as Equation (1) below.

$${sig}_{p} = 1 - beta\_cdf ( SNR , S + {prior}_{1} , N + {prior}_{2} ) \qquad ( 1 )$$

wherein S denotes the first measurement of the target antigen bound to the antigen binding molecule, prior₁ denotes a first prior probability distribution of the target antigen bound to the antigen binding molecule, N denotes the second measurement of a control antigen bound to the antigen binding molecule, and prior₂ denotes a second prior probability distribution of the control antigen bound to the antigen binding molecule.

The specificity metric sigₚ for the antigen binding molecule may correspond to a likelihood of the antigen binding molecule binding to the target antigen at above a certain signal-to-noise (SNR) threshold. It should be appreciated that the signal-to-noise threshold may be set to different values such as, for example, to 0.90, 0.91, 0.92, 0.93, 0.94, 0.95, 0.96, 0.97, 0.98, 0.99, and/or the like. For example, the value of the specificity metric sigₚ may correspond to the probability that the true value of

$$\frac{S}{( S + N )}$$

is at least a certain threshold percentage (e.g., 90%, 91%, 92%, 93%, 94%, 95%, 96%, 97%, 98%, or 99%) corresponding to the signal-to-noise threshold. The SNR threshold may be between about 0.8 and about 1.0, between about 0.85 and about 1.0, or between about 0.9 and about 1.0. In particular embodiments, the SNR threshold is between about 0.9 and about 1.0. Accordingly, the specificity metric sigₚ for the antigen binding molecule may indicate, based on the observations that a first quantity of target antigens bound to the antigen binding molecule and a second quantity of control antigens bound to the antigen binding molecule, the likelihood that the antigen binding molecule will bind to the target antigen above a threshold percentage of times. As noted, where the specificity metric sigₚ for the antigen binding molecule is high, such as when the specificity metric sigₚ for the antigen binding molecule exceeds a threshold value, the processing unit 140 may identify the antigen binding molecule as exhibiting sufficient specificity towards the target antigen. As will be described in more detail, the specificity metric sigₚ for the antigen binding molecule may vary as a result of adjusting the signal-to-noise threshold.

Referring again to Equation (1), the processing unit 140 may determine the specificity metric sigₚ for the antigen binding molecule based on the first prior probability distribution prior₁ of the target antigen bound to the antigen binding molecule and the second prior probability distribution prior₂ of the control antigen bound to the antigen binding molecule. The first prior probability distribution prior₁ and/or the second prior probability distribution prior₂ may be determined based on a summary quantity of unique molecular molecule identifiers, such as a median or mean quantity of unique molecular identifiers, present in an empty partition (e.g., a droplet in an emulsion or a well) without any cells. For example, in some example embodiments, the first prior probability distribution prior₁ and/or the second prior probability distribution prior₂, may be determined by sorting cells expressing antigen binding molecules bound to the target antigen (e.g., antigen positive cells) and cells expressing antigen binding molecules not bound to the target antigen (e.g., antigen negative cells) to generate two corresponding count distributions. The empty droplets in the antigen positive population may be used to parameterize an algorithm that enables a differentiation between authentic binders, which are antigen binding molecules that are genuinely capable of binding to the target antigen, and non-authentic binders sampled from the same distribution as the empty droplets.

Accordingly, in some cases, the first prior probability distribution prior₁ and/or the second prior probability distribution prior₂ may be determined based on at least one of a summary value (e.g., median, mean, and/or the like) of a determined measurement of the target antigen, a determined measurement of the control antigen, and a detected gene expression level in an empty partition (e.g., a droplet in an emulsion or a well) without any cells. It should be appreciated that the first prior probability distribution prior₁ and/or the second prior probability distribution prior₂ may be a normal distribution, a uniform distribution, and/or the like. Moreover, the first prior probability distribution prior₁ and/or the second prior probability distribution prior₂ may be expressed in a variety of ways including, for example, as counts (e.g., +1 or +2), ratios (10:1, 25:1, 10:2, or 25:2), and/or the like.

In some cases, the processing unit 140 may determine multiple specificity metrics, at least one (e.g., each) of which corresponding to one or more different cells of a clonotype and/or subclonotype. For example, the processing unit 140 may determine a first specificity metric for a first antibody binding molecule (ABM) expressed by a first cell of the clonotype and/or subclonotype. Furthermore, the processing unit 140 may determine a second specificity metric for a second antibody binding molecule (ABM) expressed by a second cell of the same clonotype and/or subclonotype. The antigen specificity of the clonotype and/or subclonotype may be determined based on the first specificity metric of the first antibody binding molecule expressed by the first cell and the second specificity metric of the second antibody binding molecule expressed by the second cell. For instance, the processing unit 140 may determine a summary value (e.g., a mean, a geometric mean, a median, median-of-medians, and/or the like) corresponding to the first specificity metric and the second specificity metric. Moreover, the processing unit 140 may determine, based at least on the summary value, whether the clonotype and/or the subclonotype exhibits sufficient specificity towards the target antigen.

As noted, the specificity metric sigₚ for the antigen binding molecule may vary as a result of adjusting the signal-to-noise threshold. Accordingly, in some cases, the processing unit 140 may determine multiple specificity metrics for the antigen binding molecule, at least one (e.g., each) of which being associated with a different signal-to-noise threshold. Moreover, the processing unit 140 may select, based at least on a magnitude of the differences between specificity metrics associated with different signal-to-noise thresholds, one or more of the specificity metrics for determining the antigen specificity of the antigen binding molecule. To further illustrate, Table 1 below depicts examples of the specificity metric sigₚ computed for different signal-to-noise threshold values.

TABLE 1

| | | Target | Control | Sigₚ | Sigₚ | Sigₚ |
|---|---|---|---|---|---|---|
| | Subclono- | spike | albumin | at | at | at |
| Clonotype | type | UMIs | UMIs | 0.90 | 0.95 | 0.99 |
| 1 | 1 | 184 | 0 | 100.0% | 99.8% | 54.8% |
| 2 | 1 | 122 | 3 | 99.7% | 77.2% | 1.0% |
| 2 | 2 | 61 | 6 | 40.1% | 2.3% | 0.0% |
| 3 | 3 | 234 | 20 | 80.7% | 78.4% | 11.2% |

Table 1 above shows the specificity metric sigₚ of exemplary clonotypes and subclonotypes from an experiment capturing spike-specific antibodies from a convalescent human survivor of COVID-19. For each antibody, a specificity metric sigₚ is computed for different signal-to-noise thresholds including 0.90 (90%), 0.95 (95%), and 0.99 (99%). As shown in Table 1, the specificity metric sigₚ of an antibody may decrease when the signal-to-noise threshold increases, for example, from 0.90 (90%) to 0.99 (99%). This pattern is consistent with a higher signal-to-noise threshold requiring the antibody to bind to the target antigen a higher percentage of times. For example, Table 1 shows that the specificity metric for the antibody of clonotype no. 1 and subclonotype no. 1 is 99.7%, 77.2%, and 1.0% for a signal-to-noise threshold of 0.90 (90%), 0.95 (95%), and 0.99 (99%), respectively.

In some cases, the processing unit 140 may select one or more of the specificity metrics sigₚ as representative of an actual likelihood of the antigen binding molecule binding to the target antigen based at least on the difference between the specificity metric sigₚ associated with different signal-to-noise threshold. For example, where the processing unit 140 detects a below threshold difference between a first specificity metric associated with a first signal-to-noise threshold and a second specificity metric associated with a second signal-to-noise threshold while the difference between the second specificity metric and a third specificity metric associated with a third signal-to-noise threshold is at or above the threshold, the processing unit 140 may select the first specificity metric instead of the second specificity metric or the third specificity metric as representative of an actual likelihood of the antigen binding molecule binding to the target antigen. Referring again to Table 1, the processing unit 140 may select to assess the spike protein binding specificity of the antibody of clonotype no. 1 and subclonotype no. 1 based on the specificity metric associated with a signal-to-noise threshold of 0.90 (90%) and/or the specificity metric associated with a signal-to-noise threshold of 0.95 (95%) and not the specificity metric associated with a signal-to-noise threshold of 0.99 (99%) at least because of the precipitous change in the specificity metric when the signal-to-noise threshold is increased from 0.95 (95%) to 0.99 (99%).

In some example embodiments, in addition to evaluating the antigen specificity of one or more antigen binding molecules, the processing unit 140 may further support the identification of antigen binding molecules that exhibit properties such as the presence of certain gene segments. For example, upon identifying one or more antigen binding molecules as exhibiting sufficient specificity towards a target antigen, the processing unit 140 may apply one or more filters to further select one or more antigen binding molecules that exhibit certain properties, which may include predetermined criteria that are user defined (e.g., determined based on inputs received from the client device 1506). In some cases, for instance, antigen binding molecules with sufficient binding specificity towards the target antigen may be further selected based on the presence of a certain gene sequence. Alternatively and/or additionally, antigen binding molecules with sufficient binding specificity towards the target antigen may be further selected based on the presence of a certain gene sequence within one or more specific segments of the antigen binding molecule such as the heavy chain of a B cell receptor (BCR), the light chain of a B cell receptor, the alpha chain of a T cell receptor (TCR), the beta chain of a T cell receptor, a complementarity determining region (CDR) of an immune cell receptor, a variable (V) gene segment sequence of an immune cell receptor, a joining (J) gene segment sequence of an immune cell receptor, a diversity (D) sequence of an immune cell receptor, a constant (C) sequence of an immune cell receptor, and/or the like.

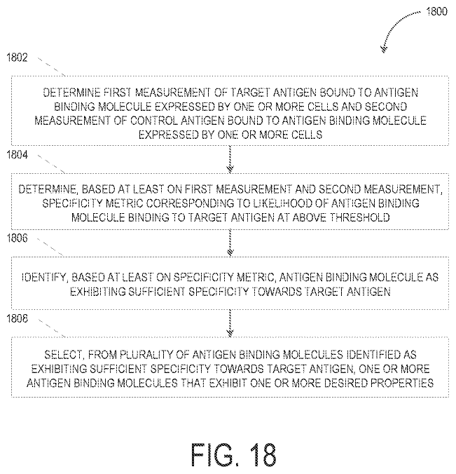

FIG. 18 depicts a flowchart illustrating an example of a process 1800 for antigen specificity analysis, in accordance with some example embodiments. The process 1800 may be performed by the processing unit 140 to assess the antigen specificity of one or more antigen binding molecules (ABMs) and to identify those exhibiting sufficient specificity towards a target antigen.

At 1802, the processing unit 140 may determine a first measurement of a target antigen bound to an antigen binding molecule expressed by one or more cells and a second measurement of a control antigen bound to the antigen binding molecule expressed by one or more cells. In some example embodiments, the processing unit 140 may determine, based on sequence dataset, e.g., a sequence dataset received from the data source 110, a first measurement of a target antigen bound to an antigen binding molecule expressed by one or more cells and a second measurement of a control antigen bound to the antigen binding molecule. The sequence dataset received from the data source 110 may be associated with a reporter oligonucleotide, which may be operatively coupled (e.g., directly or indirectly conjugated) to an antigen of a plurality of antigens to enable an identification of individual antigens and/or a differentiation between different antigens in the case of a multiplexed antigen assay. The first measurement of the target antigen bound to the antigen binding molecule may correspond to a quantity of unique molecular identifiers (UMIs) associated with the target antigen bound to the antigen binding molecule or, in the case of multiple related target antigens, a sum of the quantities of unique molecular identifiers (UMIs) associated with at least one (e.g., each) of the related individual target antigens. The second measurement of the control antigen bound to the antigen binding molecule may correspond to a quantity of unique molecular identifiers (UMIs) associated with the control antigen bound to the antigen binding molecule. Alternatively, in cases where multiple related control antigens are present, the second measurement may be a sum of the quantities of unique molecular identifiers (UMIs) associated with at least one (e.g., each) control antigen. For example, the second measurement may be a sum of a first quantity of unique molecular identifiers (UMIs) associated with a first control antigen bound to the antigen binding molecule and a second quantity of unique molecular identifiers (UMIs) associated with a second control antigen bound to the antigen binding molecule.

At 1804, the processing unit 140 may determine, based at least on the first measurement and the second measurement, a specificity metric corresponding to a likelihood that the antigen binding molecule binds to the target antigen at above a threshold. In some example embodiments, the processing unit 140 may determine, based at least on the data received form the data source 110, a specificity metric sigₚ for the antigen binding molecule (ABM). The specificity metric sigₚ for the antigen binding molecule may be determined based on a cumulative distribution function (CDF) of the beta distribution associated with the antigen binding molecule binding to the target antigen and the antigen binding molecule binding to the control antigen. Accordingly, in accordance with Equation (1), the specificity metric sigₚ of the antigen binding molecule may be determined based on the first measurement S of the target antigen bound to the antigen binding molecule, the first prior probability distribution prior₁ of the target antigen bound to the antigen binding molecule, the second measurement N of the control antigen bound to the antigen binding molecule, and the second prior probability distribution prior₂ of the control antigen bound to the antigen binding molecule. The specificity metric sigₚ for the antigen binding molecule may correspond to a likelihood of the antigen binding molecule binding to the target antigen at above a certain signal-to-noise (SNR) threshold. Adjusting the signal-to-noise (SNR) threshold may cause variations in the corresponding specificity metric sigₚ.

At 1806 the processing unit 140 may identify, based at least on the specificity metric, the antigen binding molecule as exhibiting sufficient specificity towards the target antigen. In some example embodiments, whether the antigen binding molecule exhibits sufficient specificity towards the target antigen may be determined based on the specificity metric sigₚ of the antigen binding molecule. For example, a higher specificity metric sigₚ may indicate a higher binding specificity toward the target antigen. Accordingly, when the specificity metric sigₚ of the antigen binding molecule is high, such as above a threshold value, the processing unit 140 may identify the antigen binding molecule exhibits sufficient specificity towards the target antigen.

At 1808, the processing unit 140 may select, from a plurality of antigen binding molecules identified as exhibiting sufficient specificity towards the target antigen, one or more antigen binding molecules exhibiting one or more desired properties. In some example embodiments, the processing unit 140 may analyze the antigen specificity of multiple antigen binding molecules. In some cases, upon identifying one or more antigen binding molecules as exhibiting sufficient specificity towards a target antigen, the processing unit 140 may apply one or more filters to further select one or more antigen binding molecules that exhibit certain properties such as the presence of a certain gene sequence.

As noted, the processing unit 140 may receive, from the data source 110, the sequence dataset for analyzing the antigen specificity of an antigen binding molecule (ABM) expressed by one or more cells (e.g., immune cells). In some example embodiments, the sequence dataset may be associated with a cell expressing the antigen binding molecule. The sequence dataset may be associated with the cell using a partition-specific barcode sequence. Accordingly, the processing unit 140 may determine, based at least on the sequence dataset, a first measurement of a target antigen bound to the antigen binding molecule expressed by the cell and a second measurement of a control antigen bound to the antigen binding molecule expressed by the cell.

In some example embodiments, the sequence dataset received from the data source 110 may be generated by partitioning at least a portion of a reaction mixture containing one or more cells, the target antigen, and the control antigen. The partitioning of the reaction mixture may generate multiple partitions, including the partition occupied by the aforementioned cell and associated with the partition-specific barcode sequence. In some cases, prior to the partitioning, the reaction mixture may be formed by contacting the one or more cells with the antigens. In some embodiments, the one or more cells are contacted with the antigens and a plurality of additional labelling agents. In some embodiments, the additional labeling agents are configured to bind or otherwise couple to one or more cell-surface features of an immune cell. In some example embodiments, such additional labeling agents can be used to characterize cells and/or cell features. In some example embodiments, reporter oligonucleotides of the additional labeling agents have different adapter sequences, e.g., different primer sequences or primer binding sequences, e.g., different sequencing primer sequences or sequencing primer binding sequences than reporter oligonucleotides than reporter oligonucleotides attached to target and/or non-target antigens.

In some example embodiments, the target antigen in the reaction mixture may be operatively coupled (e.g., directly or indirectly conjugated) to a first reporter oligonucleotide of a first reporter barcode sequence while the control antigen may be operatively coupled (e.g., directly or indirectly conjugated) to a second reporter oligonucleotide of a second reporter barcode sequence. The first reporter oligonucleotide, the second reporter oligonucleotide, or both the first reporter oligonucleotide and the second reporter oligonucleotide may include one or more functional sequences selected from an adapter sequence, a primer sequence, a primer binding sequence, and a unique molecular identifier (UMI). In some cases, the target antigen and/or the control antigen may be further operatively coupled (e.g., directly or indirectly conjugated) to a detectable moiety such as a mass tag, a magnetic particle, a fluorophore, and/or the like. Accordingly, in some cases, prior to the partitioning of the reaction mixture, the one or more cells may be sorted according to a flow cytometry profile based on the detectable moiety.

In some embodiments, the partitioning comprises partitioning the reaction mixture, or portion thereof, and nucleic acid barcode molecules into the plurality of partitions. In some embodiments, the partitioning provides a partition comprising the partition comprising the aforementioned cell and a plurality of nucleic acid barcode molecules comprising the partition-specific barcode sequence. In some embodiments, a nucleic acid barcode molecule comprising the partition-specific barcode sequence further comprises one or more functional sequences. The one or more functional sequences may include one or more of: an adapter sequence, a primer sequence, a primer binding sequence, and a unique molecular identifier (UMI) sequence.

A first barcoded polynucleotide may be generated using a first analyte including a nucleic acid sequence that encodes at least a portion of the antigen binding molecule expressed by the cell and a first nucleic acid barcode molecule of the plurality of nucleic acid barcode molecules comprising the partition-specific barcode sequence. The first analyte may include one or more of a variable (V) gene segment sequence, a joining (J) gene segment sequence, a diversity (D) gene segment sequence, or a constant (C) gene segment sequence of the antigen binding molecule. The first analyte may encode at least a portion of a B cell receptor (BCR) heavy chain and wherein the second analyte encodes at least a portion of a B cell receptor (BCR) light chain of the antigen binding molecule. Alternatively, the first analyte may encode at least a portion of a T cell receptor (TCR) alpha chain and wherein the second analyte encodes at least a portion of a T cell receptor (TCR) beta chain of the antigen binding molecule. The resulting first barcoded polynucleotide may include (i) a sequence of the first analyte or reverse complement thereof and (ii) the partition-specific barcode sequence or a reverse complement thereof. The sequence of the first barcoded polynucleotide (or a derivative thereof) may be determined and may form a part of the sequence dataset received from the data source 110.

In some example embodiments, the partition containing the cell expressing the antigen binding molecule (ABM) may also include the target antigen bound to the antigen binding molecule (ABM) expressed by the cell. In some cases, the partition may contain the cell expressing the target antigen but not an immune receptor (e.g., a B cell receptor (BCR) or a T cell receptor (TCR)) or a portion of an immune receptor. The sequence dataset received from the data source 110 may be further generated by using the first reporter oligonucleotide associated with the target antigen and a second nucleic acid barcode molecule of the nucleic acid barcode molecules comprising the partition-specific barcode sequence of the partition to generate a second barcoded polynucleotide. This second barcoded polynucleotide may include (i) the first reporter barcode sequence (including the first reporter oligonucleotide of the target antigen) or reverse complement thereof and (ii) the partition-specific barcode sequence or a reverse complement thereof. A sequence of the second barcoded polynucleotide (or derivative thereof) may be determined and may form a part of the sequence dataset received from the data source 110.

In some example embodiments, the partition containing the cell expressing the antigen binding molecule (ABM) may also include the control antigen bound to the antigen binding molecule (ABM) expressed by the cell. In some cases, the partition may contain the cell expressing the control antigen but not an immune receptor (e.g., a B cell receptor (BCR) or a T cell receptor (TCR)) or a portion of an immune receptor. Accordingly, the sequence dataset received from the data source 110 may be further generated using the second reporter oligonucleotide associated with the control antigen and a third nucleic acid barcode molecule of the nucleic acid barcode molecules comprising the partition-specific barcode sequence of the partition to generate a third barcoded polynucleotide. The third barcoded polynucleotide may include (i) the second reporter barcode sequence (including the second reporter oligonucleotide of the control antigen) or reverse complement thereof and (ii) the partition-specific barcode sequence or a reverse complement thereof. A sequence of the third barcoded polynucleotide (or derivative thereof) may be determined and may form a part of the sequence dataset received from the data source 110.

In some cases, the sequence dataset received from the data source 110 may be further generated using a second analyte that includes a nucleic acid sequence encoding at least a different portion of the antigen binding molecule expressed by the first cell and a fourth nucleic acid barcode molecule of the nucleic acid barcode molecules comprising the partition-specific barcode sequence of the partition to generate a fourth barcoded polynucleotide. The second analyte may include one or more of a variable (V) gene segment sequence, a joining (J) gene segment sequence, a diversity (D) gene segment sequence, or a constant (C) gene segment sequence of the antigen binding molecule. The second analyte may encode at least a portion of a B cell receptor (BCR) heavy chain and wherein the second analyte encodes at least a portion of a B cell receptor (BCR) light chain of the antigen binding molecule. Alternatively, the second analyte may encode at least a portion of a T cell receptor (TCR) alpha chain and wherein the second analyte encodes at least a portion of a T cell receptor (TCR) beta chain of the antigen binding molecule. The resulting fourth barcoded polynucleotide may include (i) a sequence of the second analyte or reverse complement thereof and (ii) the partition-specific barcode sequence or a reverse complement thereof. A sequence of the fourth barcoded polynucleotide may be determined and may be included in the sequence dataset received from the data source 110.

In some example embodiments, one or more of the aforementioned first, second, third, and fourth barcoded polynucleotides may include a unique molecular identifier (UMI) sequence or a reverse complement thereof. As such, upon receiving the sequence dataset from the data source 110, the processing unit 140 may determine the first measurement of the target antigen bound to the antigen binding molecule (ABM) expressed by the one or more cells based on a quantity of (i) unique molecular identifier (UMI) sequences or reverse complements of unique molecular identifier (UMI) sequences associated with the partition-specific barcode sequence or a reverse complement of the partition-specific barcode sequence, and (ii) the first reporter barcode sequence or a reverse complement of the first reporter barcode sequence associated with the target antigen. Moreover, the processing unit 140 may determine the second measurement of the control antigen bound to the antigen binding molecule expressed by the one or more cells based on a quantity of (i) unique molecular identifier (UMI) sequences or reverse complements of unique molecular identifier (UMI) sequences associated with the partition-specific barcode sequence or a reverse complement of the partition-specific barcode sequence, and (ii) and the second reporter barcode sequence or a reverse complement of the second reporter barcode sequence.

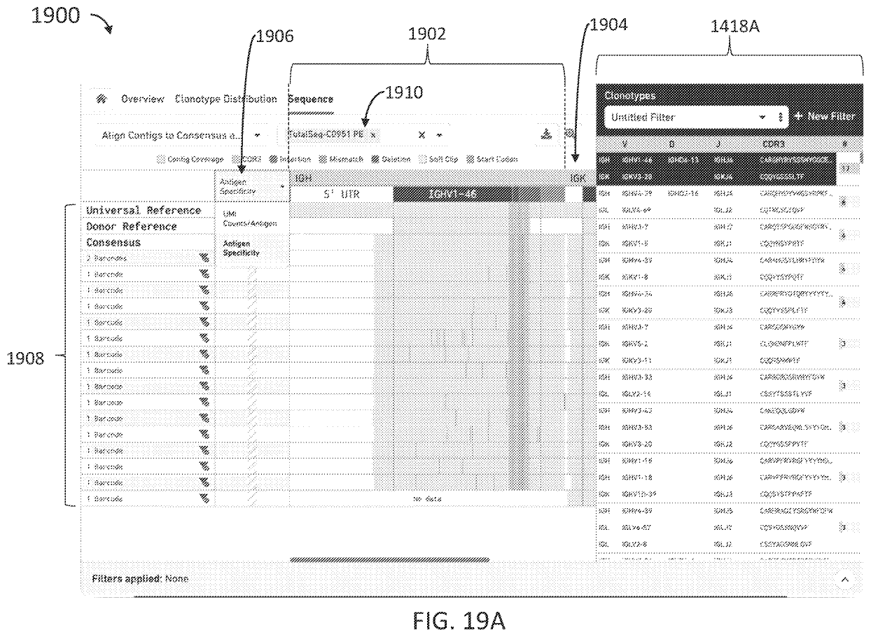

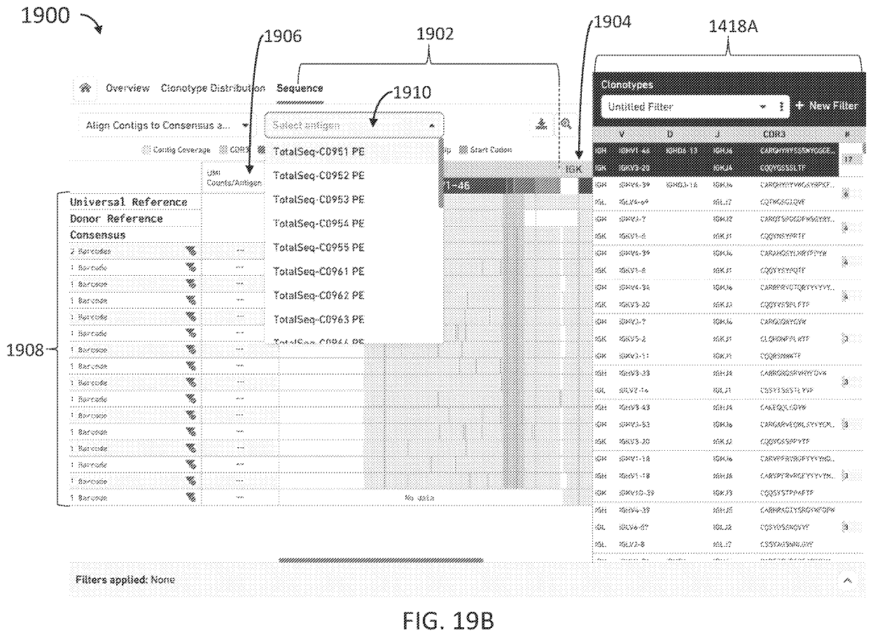

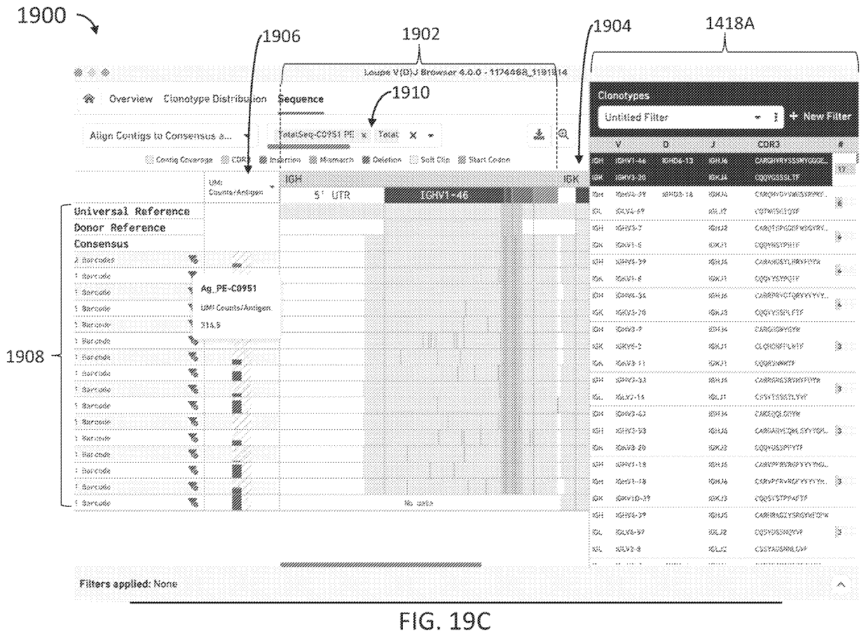

FIGS. 19A-19C illustrate an example output display 1900 of a visualization tool for cellular data. As shown in FIGS. 19A-19C, the display 1900 includes a heavy chain sequence representation 1902 identifying various features of the heavy chain of one or more clonotypes. For example, the features identified include contig coverage, CDR3, insertions, deletions, soft clip, and a start codon. In various embodiments, each feature is assigned a different color for visualization within the heavy chain sequence representation 1902. In various embodiments, the display 1900 includes a light chain sequence representation 1904 positioned adjacent to the heavy chain sequence representation 1902. The combined heavy chain representation and the light chain representation may be called more generally a chain view. The display 1900 further includes a table 1418A of clonotype information, as described in more detail above. In various embodiments, the display 1900 includes a barcode table 1908. The barcode sequence table 1908 shows the barcode sequences associated with the heavy chain representation 1902 and the light chain representation 1904. In various embodiments, barcode sequences are grouped when two or more barcodes are associated with a single heavy chain and light chain in the chain view. In various embodiments, the barcode sequence table 1908 is positioned adjacent to the heavy chain sequence representation 1902. In various embodiments, the display includes a feature selection tool 1906 that allows a user to display antigen specificity or UMI counts/antigen for each grouping of barcodes in the barcode table 1908. In various embodiments, the display 1900 includes an antigen selection tool 1910 for selecting one or more antigens. In various embodiments, a user may select one or more antigens (in some embodiments, up to a predetermined number to prevent crowding of information) for displaying associated UMI counts and/or antigen specificity of the barcodes in the barcode sequence table 1908 related to the selected one or more antigens. As described above, the user may switch between viewing UMI counts and antigen specificity via the feature selection tool 1906.

### Additional Features of Clonotype Data Visualization

In accordance with various embodiments, various features can be provided to supplement the various embodiments provided herein.

As stated above, visualization of identified clonotypes can source from single cell datasets. Mechanisms for calling specific datasets can originate from various sources that include, for example, entering the data source path directly on the command line, or via a supplementary metadata file.

When entering the data source path directly on the command line, a common entry simply points at specific input files. For a more complicated syntax, punctuation can be used such as, for example, commas, colons and semicolons that can act as delimiters. Commas can be used, for example, between datasets from the same sample. Colons can be used, for example, between datasets from the same donor. Semicolons can be used to separate donors. Using this input system, each dataset can be assigned an abbreviated name, which can be everything after the final slash in the directory name. The entire name of a dataset can be used, for example, when there is no slash. Moreover, samples and donors can be assigned numerical identifiers starting at one. Using this system, a base example of input data from two libraries from the same sample can be exemplified (e.g., TCR=p1,p2), an example of the same input data plus another from a different sample from the same donor can be exemplified (e.g., TCR=p1,p2:q), and example of input data of one library from each of two donors can be exemplified (e.g., TCR=“a;b”). Likewise, matching gene expression and/or feature barcode data may also be supplied using an argument “GEX= . . . ”.

To specify a metadata file, as opposed to entering a data source directly on the command line, a user can implement a specific command line argument calling a metadata file (e.g, META=filename). The file can be in a CSV format (comma-separated values) or tab-separated/character-delimited data format. In addition to the metadata file call, other fields can be used to provide further parameters. For example, a field such as “tcr” or “bcr” can be used to provide a path to the dataset, wherein the full file name can be used or an abbreviated name for the data set can be used, generally with a designation that an abbreviated name is being used (e.g., “abbr”). Further, a field such as “gex” can be used to provide a path to the gene expression dataset, which may include of consist of a function-based (FB) dataset. Further fields such as, for example, “sample” or “donor” can be used to provide a name, or abbreviated name of a sample or donor respectively.

When specifying a CDR sequence in the command line, the sequence can be input various ways. For example, one could require an exact sequence (e.g., CDR3=CARPKSDYIIDAFDIW), at least one of multiple sequences (e.g., CDR3=“CARPKSDYIIDAFDIWICQVWDSSSDHPYVF”), or a snippet of a sequence inside the CDR sequence (e.g., “.*DYIID.*”), where quotations are used when non-letter characters are provided (e.g., “.”, “*”, “|”).

In accordance with various embodiments, the output visualization can be customized in a variety of ways to provide the user desired targeted output information and augment the output. Customization can be based on, for example, cell count, unique-molecular-identifier (UMI) count, chain count, CDR (e.g., CRD3) patterns, V(D)J segment specification, subclonotype count, VJ segment specification, cross-data set cell comparisons, universal reference comparisons, deletion antigen specificity, specificity, or other clonotype/subclonotype/barcode-specific information provided as metadata in parallel to the application.

For cell count customization, fields can be used to show clonotypes having at least n cells (e.g., MIN_CELLS=n), show clonotypes having at most n cells (e.g., MAX_CELLS=n), or show clonotypes having exactly n cells (e.g., CELLS=n). For UMI count customization, fields can be used to show clonotypes having ≥ n UMIs on some chain on some cell (e.g., MIN-UMIS=n).

For chain count customization, fields can be used to show clonotypes having at least n chains (e.g., MIN_CHAINS=n), show clonotypes having at most n chains (e.g., MAX_CHAINS=n), show clonotypes having exactly n chains (e.g., CHAINS=n). For CDR patterns, fields can be used to show clonotypes having a CDR3 amino acid sequence that matches a given pattern, from beginning to end (e.g., CDR3=<pattern>).

For V(D)J segment specification, fields can be used to show clonotypes using one of the given VDJ segment names (double quotes can be used if n>1) (e.g., “SEG=s_1| . . . |s_n”), or show clonotypes using one of the given VDJ segment numbers (double quotes only needed if n>1) (e.g., “SEGN=s_1| . . . |s_n”).

For subclonotype count specification, fields can be used to show clonotypes having at least n exact subclonotypes (e.g., MIN_EXACTS=n). For VJ segment specification, fields can be used to show clonotypes using exactly the given V . . . J sequence (string in alphabet ACGT) (e.g., VJ=seq).

For cross-data set cell comparisons, fields can be used to show clonotypes containing cells from at least n datasets (e.g., MIN_DATASETS=n). For universal reference comparisons, fields can be used to show clonotypes having a difference in constant region with the universal reference (e.g., CDIFF). For deletion specificity, fields can be used to show clonotypes exhibiting a deletion (e.g., DEL).

In accordance with various embodiments, the output visualization can be customized with a variety of filtering options to provide the user desired targeted output information and augment the output. These filtering options could include turning on a filter or turning off a filter.

In accordance with various embodiments, the output visualization can be customized with a variety of options to suppress or display additional output. An example of an output option is an export filter. If one specifies that export of the donor-derived reference, FASTA nucleotide sequence of an exact subclonotype, FASTA amino acid sequence of an exact subclonotype, or of a selection of any or a subset of the fields generated by analysis should be performed, then these features can be displayed and simultaneously written to a user-specified file in the appropriate format.

An example of a filtering option is a cross-filter. If one specifies that two or more libraries arose from the same sample (i.e., from the same tube of cells), then the default behavior of the various embodiments herein, can be to “cross filter” so as to remove expanded exact subclonotypes that are present in one library but not another, in a fashion that would be highly improbable, assuming random draws of cells from the tube. Such observed behavior can be understood to arise when a plasma or plasmablast cell breaks up during or after pipetting from the tube, and the resulting fragments seed can yield ‘fake’ cells. This filter, presumably defaulted to being on during sample analysis of subclonotype identification, can also be turned off per user input. It is understood that the reverse is also contemplated.

Another example of a filtering option relates to a filter that, by default in various embodiments, removes exact subclonotypes that by virtue of their relationship to other exact subclonotypes, appear to arise from background mRNA or a phenotypically similar phenomenon. This filter, presumably defaulted to being on during sample analysis of subclonotype identification, can also be turned off per user input. It is understood that the reverse is also contemplated.

Another example of a filtering option relates to a filter that, by default in various embodiments, filters out exact subclonotypes having a base in V(D)J sequence that looks like it might be wrong. A Phred quality score (Q score) is a measure of the quality of the identification of the nucleobases generated by automated DNA sequencing. Various methods, in accordance with various embodiments herein, can find bases which are not Q60 for a barcode, not Q40 for two barcodes, are not supported by other exact subclonotypes, are variant within the clonotype, and which disagree with the donor reference. This filter, presumably defaulted to being on during sample analysis of subclonotype identification, can also be turned off per user input. It is understood that the reverse is also contemplated.

Another example of a filtering option relates to a filter that, by default in various embodiments, filters out chains from clonotypes that are weak and appear to be artifacts, perhaps arising from, for example, a stray mRNA molecule. This filter, presumably defaulted to being on during sample analysis of subclonotype identification, can also be turned off per user input. It is understood that the reverse is also contemplated.

Another example of a filter relates to a filter that, by default in various embodiments, identifies and filters out cells with low credibility, or barcode-associated rearrangements that artificially inflate the size of a given clonotype. This filter operates by using V(D)J sequence data in addition to one or more modes of data for the same cells. This filter is comprised of multiple steps, each of which can be run independently or in combinations with any of the other steps. These steps may include: (1) removal of V(D)J cells and chains that are not present in the second dataset (for example, remove of V(D)J cells if those cells are not also found in the orthogonal gene expression dataset); (2) for a clonotype of n cells, determining for each cell in the clonotype, the n nearest neighbors in an appropriate dimensional reduction or using a sensible distance metric to find these neighbors' gene expression or other dataset; and (3) calculating the credibility of a cell, where credibility is the percent of those nearest neighbors meeting at least one or more of the following criteria: (a) where the nearest neighbors are also V(D)J-called cells, (b) where the nearest neighbors are immune cells, e.g., B or T cells, identified by supervised analysis, (c) where the nearest neighbors are immune cells, e.g., B or T cells identified by supervised analysis, and (d) where the nearest neighbors are a non-B or non-T cell or a cell that should not otherwise express a B or T cell receptor. This filter can also use the nearest neighbor graph from various clustering algorithms (e.g. the Leiden or Louvain algorithms, and other commonly known algorithms) to calculate credibility of cells by: (1) measuring the geodesic distance between a cell and its n nearest neighbors in the graph; and (2) determining which of those nearest neighbors meet the comparison criteria listed above. This filter, presumably defaulted to being on for identifying and filtering out cells with low credibility, or barcode-associated rearrangements that artificially inflate the size of a given clonotype, can also be turned off per user input. It is understood that the reverse is also contemplated.

Another example of a filtering option relates to a filter that, by default in various embodiments, filters out onesie clonotypes (a clonotype or exact subclonotype having exactly one chain) having a single exact subclonotype, and that are light chain or TRA gene, and whose number of cells is less than, for example, 0.1% of the total number of cells. This filter, presumably defaulted to being on during sample analysis of subclonotype identification, can also be turned off per user input. It is understood that the reverse is also contemplated.

Another example of a filtering option relates to a filter that, by default in various embodiments, finds a foursie exact subclonotype that contains a twosie exact subclonotype having at least ten cells, it kills the foursie exact subclonotype, no matter how many cells it has. The foursies that are killed are believed to be rare odd artifacts arising from repeated cell doublets or, for example, GEMs (Gel bead-in-EMulsion) that contain two cells and multiple gel beads. This filter, presumably defaulted to being on during sample analysis of subclonotype identification, can also be turned off per user input. It is understood that the reverse is also contemplated.

Another example of a filtering option relates to a filter that, by default in various embodiments, filters out rare artifacts arising from contamination of oligos on gel beads. This filter, presumably defaulted to being on during sample analysis of subclonotype identification, can also be turned off per user input. It is understood that the reverse is also contemplated.

Another example of a filtering option relates to a filter that, by default in various embodiments, labels an exact subclonotype as improper if it does not have one chain of each type. This filtering option causes all improper exact subclonotypes to be retained, although they may be removed by other filters.

Another example of a filter relates to a filter that, by default in various embodiments, can be used to select exact subclonotypes within a specified range of generation probability, where the generation probability is calculated by calculating the likelihood of a specific rearrangement being generated relative to rearrangements generated in silico. In some embodiments, the generation probability is conditioned on the V gene used in the observed rearrangement. In some embodiments, spurious subclonotypes that may have been identified by de novo assembly or that arose due to chemistry errors can be removed by application of this filter in combination with other filters described. This filter, presumably defaulted to being on during sample analysis of exact subclonotype identification, can also be turned off per user input. It is understood that the reverse is also contemplated

Yet another example of a filtering option relates to a filter that, by default in various embodiments, deletes any exact subclonotype having less than n chains. Such a filter can be used to “purify” a clonotype so as to display only exact subclonotypes having all their chains. Similarly, another example of a filtering option relates to a filter that, by default in various embodiments, deletes any exact subclonotype having less than n cells. Such a filter can be used for a very large and complex expanded clonotype, for which it may be desired to see a simplified view.

In accordance with various embodiments, the output visualization can be customized with a variety of lead variable and per-chain variable options to provide the user desired targeted output information and augment the output. Lead variable options (LVARS) can be formatted to appear once for each clonotype and, as shown in FIG. 2, can be provided along the left, side, with one entry for each subclonotype row. FIG. 2, shows LVARS as “gex-med”, “IGHV2-5_g” and “CD4_a”. LVARS can be specified in the example format LVARS=x1, . . . xn. The variable x can be related to datasets, donors, cells, gene expression UMI count, Hamming distance, gene expression data, and feature barcode data.

Regarding datasets and donors, a lead variable referencing donor or dataset identifiers can be used. Regarding cells, lead variables can be used that (a) provide an n number of cells or (b) provide an n number of cells associated to a given name, which can be, for example, a dataset short name, a sample short name, a donor short name, and so on. Regarding gene expression UMI count, lead variables can be use that request a median gene expression UMI count or a max gene expression UMI count. Regarding Hamming distance, lead variables can be used that request a Hamming distance of a V . . . J DNA sequence to its nearest neighbor and a V . . . J DNA sequence to its farthest neighbor. Another example using Hamming distance involves grouping all exact subclonotypes according to the Hamming distance of their V . . . J sequences. More specifically, those within distance d are defined to be in the same group, and this is extended transitively. A group identifier 1, 2, etc. can be provided, the order of which can be arbitrary. Hamming distance comparisons can be usefully applied in various situations such as, for example, cases where all exact subclonotypes have a complete set of chains. Regarding feature barcode data, lead variables can be used that (a) assume that feature barcode data has been provided, (b) look for a feature line that starts with the given name, and (c) then has a tab—the report out being in the form of mean UMI count value. Regarding gene expression data, lead variables can be used that (a) assume that gene expression data has been provided, and (b) look for a feature line that starts with the given name in the second tab delimited column—the report out being in the form of man UMI count value. In accordance with various embodiments, default LVARS can be, for example, dataset identifiers and n number of cells.

Regarding per-chain variable options (CVARS), these options define per-chain variables, which correspond to columns that appear once for each chain in each clonotype, and have one entry for each exact subclonotype. CVARS can be specified in the example format CVARS=x1, . . . xn. The variable x can be related to varying bases in chain (e.g., bases at positions in chain that vary across the clonotype), UMI counts, read counts (median VDJ read count for each exact subclonotype), constant region name, a measure of CDR3 complexity, CDR3_DNA sequence, various sequence lengths and differences, optional notes (optional note if there is an insertion, omitted if empty), and base differences (number of base differences within V . . . J with exact subclonotype n).

Regarding UMI counts, CVARS can be used that request median VDJ UMI count for each exact subclonotype, max VDJ UMI count for each exact subclonotype, or total VDJ UMI count for each exact subclonotype. Regarding various sequence lengths and differences, CVARS can be used that requests length of observed constant sequence (usually truncated at primer start) or length of observed 5′-UTR sequence. CVARS can be used that requests differences versus a universal reference constant region, which can be shown in the abbreviated form e.g. 22T (ref changed to T at base 22) or 22T+10 (same but contig has 10 additional bases beyond end of ref C region). In accordance with various embodiments, default CVARS can be, for example, median VDJ UMI count for each exact subclonotype, constant region name and optional notes (optional note if there is an insertion, omitted if empty).

In accordance with various embodiments, the output visualization can be customized with a variety of amino acid related variables (AMINO) to provide the user desired targeted output information and augment the output. There is a complex per-chain column that can be to the left of other per-chain columns, and can be specified according to the entry AMINO=x1, . . . , xn, which can result in the display of amino acid columns for the given categories, in one combined ordered group. The categories x can be one or more of CDR3 sequence, positions in chain that vary across the clonotype, positions in chain that differ consistently from the donor reference, positions in chain where the donor reference differs from the universal reference, and positions in chain where the donor reference differs non-synonymously from the universal reference.

In accordance with various embodiments, the output visualization can be customized with a variety of display options for controlling clonotype display, which can provide the user desired targeted output information and augment the output. One option is a per barcode expansion, where each exact clonotype line is expanded, showing one line per barcode, for each such line, displaying the barcode name, the number of UMIs assigned, and the gene expression UMI count, if applicable, under gex_med (see above). Another option is a barcode list, whereby a list of all barcodes of the cells in each clonotype is printed in a single line near the top of the printout for a given clonotype. Another option is to print the V . . . J sequence for each chain in the first exact subclonotype, near the top of the printout for a given clonotype. Another option is to print the full sequence for each chain in the first exact subclonotype, near the top of the printout for a given clonotype. An option for controlling clonotype grouping is to group clonotypes by perfect identity of CDR3 amino acid sequence of IGH or TRB, or group by minimum number of clonotypes in group to print.

In accordance with various embodiments, the output visualization can be customized with a variety of options handling insertions and deletions, which can provide the user desired targeted output information and augment the output. The various embodiments described herein can be configured to recognize and display a single insertion or deletion in a contig relative to the reference. Such recognition and display can be subject to standards, such as the indel length being divisible by three, being relatively short, and occurring within the V segment, but not too close to its right end. These indels can be germline, however most such events are already captured in a reference sequence. Deletions can be displayed using hyphens (-). If the var option for CVARS (see above) is used, the hyphens can be displayed in base space, where they are initially observed. For the AMINO option (see above), the deletion can be first shifted by up to two bases, so that the deletion starts at a base position that is divisible by three. The deleted amino acids can be shown as hyphens. Insertions can be shown in amino acid space, in a special per-chain column that appears if there is an insertion. Colored amino acids are shown for the insertion, and the position of the insertion can be shown. The position is the position of the amino acid after which the insertion appears, where the first amino acid (start codon) is numbered 0.

### Dynamic Filtering

It is well known that identifying sequences of, for example, T-cell receptors and antibody binding sites that bind to specific antigens is a non-trivial task. While measures such as binding specificity can empower users to funnel to sequences of interest, identifying a few sequences from a list of thousands of sequences using different VDJ sequence parameters can be cumbersome and can require multiple iterations and looking at the data through multiple lenses. Certain command-line tools can allow the flexibility to users to apply multiple filters to sequences and identify their sequences of interest.

However, given the process can be very iterative, a GUI-based workflow that allows users to understand their entire dataset, by visualization, and dynamically filter out sequences to narrow down a given list to a particular sequence (or sequences) of interest is very valuable to researchers (e.g., immunology researchers).

In general, all data used for filtering is included in the data set contained in the multi-section data file described above. In at least some embodiments, the one exception is filtering based on a gene expression category. In such embodiments, the data corresponding to gene expression category may be stored in a separate file. To filter based on gene expression category, barcodes matching the category are extracted from the separate file and used to filter data from the multi-section data file.

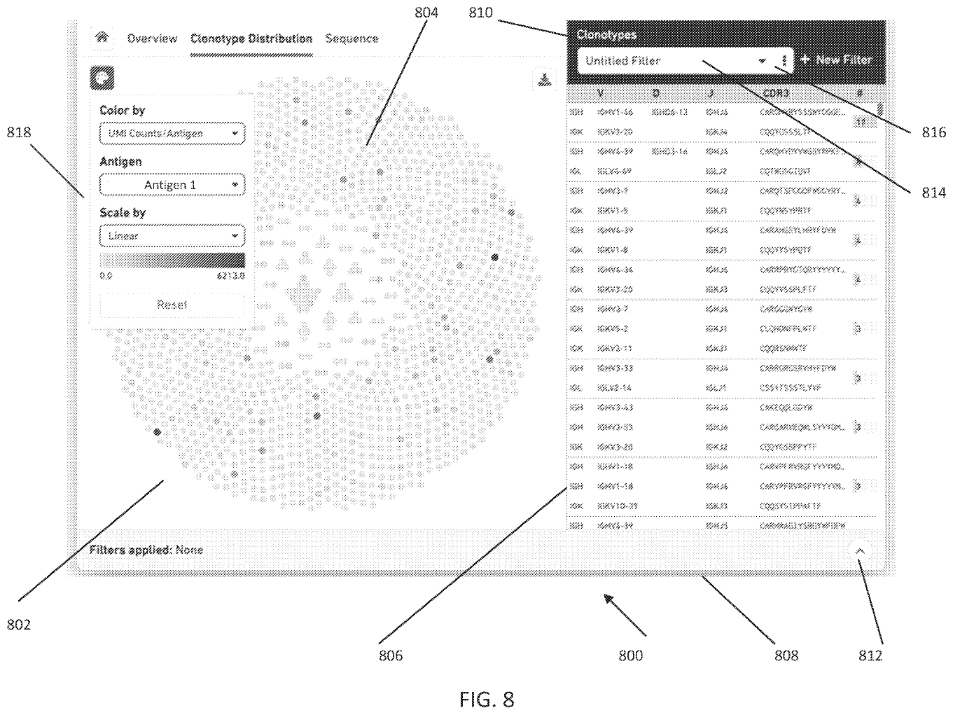

It should be noted that various embodiments of methods, processes, series of instructions, and so on, will be presented. In describing the various embodiments, the specification may present a method and/or process as a particular sequence of steps. However, the method or process should not be limited to the particular sequence of steps described, and one skilled in the art can readily appreciate that the sequences may be varied and still remain within the spirit and scope of the various embodiments. Additionally, while the dynamic filtering disclosed herein is described in relation to example illustrations (FIGS. 8-11B) showing the clonotype distribution view, it will be appreciated that any and all features of the dynamic filtering are applicable in a similar way to the sequence view (FIGS. 13-17) as well. For instance, when a filter is applied, the table 1418A and the barcode listing 1420 may be modified as a result of the filtering. In an example, the lettering of the barcodes on the barcode listing 1420 that are filtered out may change colors (e.g., grayed out).

In accordance with various embodiments, FIG. 6 illustrates a non-limiting example of a series of instructions 600, executable by a processor, for a visualization tool for cellular data. The instructions 600 can include a first operation 610, comprising receiving, by the processor, a data set comprising cellular data, e.g., a B cell receptor and/or T cell receptor data associated with a plurality of cells, e.g., a plurality of immune cells as described herein.

The instructions 600 can further include a second operation 620, comprising receiving, by the processor, a filter selection, wherein the filter is selected from the group consisting of UMI Counts/antigen, antigen specificity, barcode, number of barcodes representing a clonotype, CDR amino acid sequence (e.g., CDR3 amino acid sequence), CDR bases (e.g., CDR3 bases), gene name, isotype, cluster, amino acid % mutation, nucleotide % mutation, iNKT/MAIT, and combinations thereof.

It must be noted that any list of filters provided herein is non-limiting. For example, though CDR3-based filters are provided in the above filter list, filters based on any complementarity-determining regions (CDR1, CDR2 and CDR3) are available for use herein. As such, the filter can be selected from the group consisting of UMI Counts/antigen, antigen specificity, barcode, number of barcodes representing a clonotype, a CDR amino acid sequence, CDR bases, gene name, isotype, cluster, amino acid % mutation, nucleotide % mutation, iNKT/MAIT, and combinations thereof.

The instructions 600 can further include a third operation 630, comprising presenting an end user with a visualization tool. The visualization tool can provide a dynamic display of the data set by, for example, fourth operation 640 and fifth operation 650.

For fourth operation 640, the visualization tool can provide a dynamic display of the data set by generating, by the processor, a first plot from the data set, and displaying the first plot.

For fifth operation 650, the visualization tool can provide a dynamic display of the data set by modifying, by the processor, the first plot based on the filter selection, to generate a modified first plot that is different from the first plot, and displaying the modified first plot. The dynamic display of the data set can provide for analysis of the cellular data from the data set. Further detailed discussion regarding the various features of the dynamic display will be provided below.

As stated above, it should be understood that the processes discussed herein are not subject to the particular order of steps described. As an example of that, and in accordance with various embodiments, another non-limiting example of a computer-readable storage medium encoded with a series of instructions 600 is provided, executable by a processor, for a visualization tool for cellular data. The instructions 600 can include a first operation (see, e.g., 610 in FIG. 6), comprising receiving, by the processor, a data set comprising cellular data, e.g., a B cell receptor and/or T cell receptor data associated with a plurality of cells, e.g., a plurality of immune cells as described herein. The instructions 600 can further include a second operation (see, e.g., 630 in FIG. 6), comprising presenting an end user with a visualization tool. The instructions can further include a third operation (see, e.g., 640 in FIG. 6), where the visualization tool can provide a dynamic display of the data set by generating, by the processor, a first plot from the data set, and displaying the first plot. The instructions can further include a fourth operation (see, e.g., 620 in FIG. 6), comprising receiving, by the processor, a filter selection, wherein the filter is selected from the group consisting of UMI Counts/antigen, antigen specificity, barcode, number of barcodes representing a clonotype, CDR amino acid sequence (e.g., CDR3 amino acid sequence), CDR bases (e.g., CDR3 bases), gene name, isotype, cluster, amino acid % mutation, nucleotide % mutation, iNKT/MAIT, and combinations thereof. The instructions can further include a fifth operation (see, e.g., 650 in FIG. 6), wherein the visualization tool can provide a dynamic display of the data set by modifying, by the processor, the first plot based on the filter selection, to generate a modified first plot that is different from the first plot, and displaying the modified first plot. The dynamic display of the data set can provide for analysis of the cellular data from the data set.

Further, in accordance with various embodiments of a computer-readable storage medium encoded with instructions, executable by a processor, for a visualization tool for cellular data, the instructions can further comprise receiving, by the processor, a plurality of filter selections, wherein each filter is selected from the group consisting of UMI Counts/antigen, binding specificity (also referred to herein as antigen specificity), barcode, number of barcodes representing a clonotype, CDR amino acid sequence (e.g., CDR3 amino acid sequence), CDR bases (e.g., CDR3 bases), gene name, isotype, cluster, amino acid % mutation, nucleotide % mutation, iNKT/MAIT, and combinations thereof.

In addition, in accordance with various embodiments of a computer-readable storage medium encoded with instructions, executable by a processor, for a visualization tool for cellular data, the instructions can further comprise receiving, by the processor, a plurality of filter selections, wherein each filter is selected from the group consisting of UMI Counts/antigen, binding specificity, barcode, number of barcodes representing a clonotype, isotype, cluster, amino acid % mutation, nucleotide % mutation, iNKT/MAIT, and combinations thereof.

Further, in accordance with various embodiments of a computer-readable storage medium encoded with instructions, executable by a processor, for a visualization tool for cellular data, the cellular data can include T-Cell data. The cellular data can include B-Cell data.

Additionally, in accordance with various embodiments of a computer-readable storage medium encoded with instructions, executable by a processor, for a visualization tool for cellular data, at least one of the first plot and modified first plot can comprise a clonotype distribution plot.

As well, in accordance with various embodiments, the instructions can further comprise generating, by the processor, a first table of information from the data set, and displaying the table, and modifying, by the processor, the first table based on the filter selection, to generate a modified first table of information that is different from the first table, and displaying the modified first table, wherein at least one of the first table and modified first table comprises genetic information for identified clonotypes or cells belonging to a clonotype presented in the at least one of the first or modified first plots. The genetic information can be selected from the group consisting or V-gene, D-gene, J-gene, CDR sequence, constant region sequence, framework region (FWR) sequence, and combinations thereof.

Further, in accordance with various embodiments of a computer-readable storage medium encoded with instructions, executable by a processor, for a visualization tool for cellular data, the filter can be selected from multiple properties of VDJ sequences for heavy and light chains. Moreover, the filter can be selected from the group consisting of UMI Counts/antigen, antigen specificity, barcode, number of barcodes representing a clonotype, isotype, cluster, amino acid % mutation, nucleotide % mutation, iNKT/MAIT information, and combinations thereof.

Moreover, in accordance with various embodiments of a computer-readable storage medium encoded with instructions, executable by a processor, for a visualization tool for cellular data, the first plot can comprise a plurality of indicia, wherein an indicia of the plurality of indica represents a cell. In some embodiments, each indicia represents a cell. The instructions can further comprise modifying, by the processor, the first plot based on the filter selection, to generate the modified first plot that is different from the first plot, and displaying the modified first plot, wherein the first plot can be modified such that an indicia gets modified when a cell, subclonotype, or clonotype corresponding to the indicia does not pass a criteria of the selected filter. The instructions can further comprise modifying, by the processor, the first plot based on the filter selection, to generate the modified first plot that is different from the first plot, and displaying the modified first plot, wherein the first plot is modified such that an indicia gets modified when a cell, subclonotype, or clonotype corresponding to the indicia does pass a criteria of the selected filter. Moreover, the indicia can have a first color, and the indicia can change to a second color if modified according to the criteria of the selected filter.

Further, in accordance with various embodiments of a computer-readable storage medium encoded with instructions, executable by a processor, for a visualization tool for cellular data, the filter selection received can include a selected feature, and at least one tunable parameter of the selected feature. Also, the filter selection received can include a selected feature, and more than one tunable parameter of the selected feature.

In accordance with various embodiments, a computer implemented method for a visualization tool for cellular data is provided. The method can comprise a series of steps or operations, similar to the instructions 600 (and associated operations 610 to 650) illustrated in FIG. 6. For example, the method can comprise receiving, by the processor, a data set comprising cellular data (see, e.g., operation 610). The method can further comprise receiving, by the processor, a filter selection, wherein the filter is selected from the group consisting of UMI Counts/antigen, antigen specificity, barcode, number of barcodes representing a clonotype, CDR amino acid sequence (e.g., CDR3 amino acid sequence), CDR bases (e.g., CDR3 bases), gene name, isotype, cluster, amino acid % mutation, nucleotide % mutation, INKT/MAIT, and combinations thereof (see, e.g., operation 620). The method can further comprise presenting an end user with a visualization tool (see, e.g., operation 630). The visualization tool can provide a dynamic display of the data set by generating, by the processor, a first plot from the data set (see, e.g., operation 640), and displaying the first plot, and modifying, by the processor, the first plot based on the filter selection, to generate a modified first plot that is different from the first plot, and displaying the modified first plot (see, e.g., operation 650). The dynamic display of the data set can provide for analysis of the cellular data from the data set. Further detailed discussion regarding the various features of the dynamic display will be provided below.

Further, in accordance with various embodiments for a computer implemented method of a visualization tool for cellular data, the method can further comprise receiving, by the processor, a plurality of filter selections, wherein each filter is selected from the group consisting of UMI Counts/antigen, binding specificity, barcode, number of barcodes representing a clonotype, CDR amino acid sequence (e.g., CDR3 amino acid sequence), CDR bases (e.g., CDR3 bases), gene name, isotype, cluster, amino acid % mutation, nucleotide % mutation, iNKT/MAIT, and combinations thereof.

In addition, in accordance with various embodiments for a computer implemented method of a visualization tool for cellular data, the method can further comprise receiving, by the processor, a plurality of filter selections, wherein each filter is selected from the group consisting of UMI Counts/antigen, binding specificity, barcode, number of barcodes representing a clonotype, isotype, cluster, amino acid % mutation, nucleotide % mutation, iNKT/MAIT, and combinations thereof.

Further, in accordance with various embodiments for a computer implemented method of a visualization tool for cellular data, the cellular data can include T-Cell data. The cellular data can include B-Cell data.

Additionally, in accordance with various embodiments for a computer implemented method of a visualization tool for cellular data, at least one of the first plot and modified first plot can comprise a clonotype distribution plot.

As well, in accordance with various embodiments, the method can further comprise generating, by the processor, a first table of information from the data set, and displaying the table, and modifying, by the processor, the first table based on the filter selection, to generate a modified first table of information that is different from the first table, and displaying the modified first table, wherein at least one of the first table and modified first table comprises sequence-related information for identified clonotypes or cells belonging to a clonotype presented in the at least one of the first or modified first plots. The sequence-related information can be selected from the group consisting or V-gene, D-gene, J-gene, CDR sequence, and combinations thereof.

Further, in accordance with various embodiments for a computer implemented method of a visualization tool for cellular data, the filter can be selected from multiple properties of VDJ sequences for heavy and light chains. Moreover, the filter can be selected from the group consisting of UMI Counts/antigen, antigen specificity, barcode, number of barcodes representing a clonotype, isotype, cluster, amino acid % mutation, nucleotide % mutation, iNKT/MAIT information, and combinations thereof.

Moreover, in accordance with various embodiments of a computer implemented method for a visualization tool for cellular data, the first plot can comprise a plurality of indicia, wherein each indicia represents a cell. The method can further comprise modifying, by the processor, the first plot based on the filter selection, to generate the modified first plot that is different from the first plot, and displaying the modified first plot, wherein the first plot can be modified such that an indicia gets modified when a cell, subclonotype, or clonotype corresponding to the indicia does not pass a criteria of the selected filter. The method can further comprise modifying, by the processor, the first plot based on the filter selection, to generate the modified first plot that is different from the first plot, and displaying the modified first plot, wherein the first plot is modified such that an indicia gets modified when a cell, subclonotype, or clonotype corresponding to the indicia does pass a criteria of the selected filter. Moreover, the indicia can have a first color, and the indicia can change to a second color if modified according to the criteria of the selected filter.

Further, in accordance with various embodiments of a computer implemented method for a visualization tool for cellular data, the filter selection received can include a selected feature, and at least one tunable parameter of the selected feature. Also, the filter selection received can include a selected feature, and more than one tunable parameter of the selected feature.

In accordance with various embodiments, a system is disclosed. The system can comprise a processor and a memory in communication with the processor. The memory can store a series of instructions, steps or operations, similar to the instructions 600 (and associated operations 610 to 650) illustrated in FIG. 6. The memory can store instructions for receiving, by the processor, a data set comprising cellular data (see, e.g., operation 610). The memory can store instructions for receiving, by the processor, a filter selection, wherein the filter is selected from the group consisting of UMI Counts/antigen, antigen specificity, barcode, number of barcodes representing a clonotype, CDR amino acid sequence (e.g., CDR3 amino acid sequence), CDR bases (e.g., CDR3 bases), gene name, isotype, cluster, amino acid % mutation, nucleotide % mutation, iNKT/MAIT, and combinations thereof (see, e.g., operation 620). The memory can store instructions for presenting an end user with a visualization tool (see, e.g., operation 630). The visualization tool can provide a dynamic display of the data set by generating, by the processor, a first plot from the data set, and displaying the first plot (see, e.g., operation 640), and modifying, by the processor, the first plot based on the filter selection, to generate a modified first plot that is different from the first plot, and displaying the modified first plot (see, e.g., operation 650). The dynamic display of the data set can provide for analysis of the cellular data from the data set. Further detailed discussion regarding the various features of the dynamic display will be provided below.

Further, in accordance with various embodiments of a system, the system can further store instructions for receiving, by the processor, a plurality of filter selections, wherein each filter is selected from the group consisting of UMI Counts/antigen, binding specificity, barcode, number of barcodes representing a clonotype, CDR amino acid sequence (e.g., CDR3 amino acid sequence), CDR bases (e.g., CDR3 bases), gene name, isotype, cluster, amino acid % mutation, nucleotide % mutation, iNKT/MAIT, and combinations thereof.

Further, in accordance with various embodiments of a system, the system can further store instructions for receiving, by the processor, a plurality of filter selections, wherein each filter is selected from the group consisting of UMI Counts/antigen, binding specificity, barcode, number of barcodes representing a clonotype, CDR amino acid sequence (e.g., CDR3 amino acid sequence), CDR bases (e.g., CDR3 bases), gene name, isotype, cluster, amino acid % mutation, nucleotide % mutation, iNKT/MAIT, and combinations thereof.

Further, in accordance with various embodiments of a system, the cellular data can include T-Cell data. The cellular data can include B-Cell data.

Additionally, in accordance with various embodiments of a system, at least one of the first plot and modified first plot can comprise a clonotype distribution plot.

As well, in accordance with various embodiments, the system can further store instructions for generating, by the processor, a first table of information from the data set, and displaying the table, and modifying, by the processor, the first table based on the filter selection, to generate a modified first table of information that is different from the first table, and displaying the modified first table, wherein at least one of the first table and modified first table comprises genetic information for identified clonotypes or cells belonging to a clonotype presented in the at least one of the first or modified first plots. The genetic information can be selected from the group consisting or V-gene, D-gene, J-gene, CDR sequence, and combinations thereof.

Further, in accordance with various embodiments of a system, the filter can be selected from multiple properties of VDJ sequences for heavy and light chains. Moreover, the filter can be selected from the group consisting of UMI Counts/antigen, antigen specificity, barcode, number of barcodes representing a clonotype, isotype, cluster, amino acid % mutation, nucleotide % mutation, iNKT/MAIT information, and combinations thereof.

Moreover, in accordance with various embodiments of a system, the first plot can comprise a plurality of indicia, wherein each indicia represents a cell. The system can further store instructions for modifying, by the processor, the first plot based on the filter selection, to generate the modified first plot that is different from the first plot, and displaying the modified first plot, wherein the first plot can be modified such that an indicia gets modified when a cell, subclonotype, or clonotype corresponding to the indicia does not pass a criteria of the selected filter. The system can further store instructions for modifying, by the processor, the first plot based on the filter selection, to generate the modified first plot that is different from the first plot, and displaying the modified first plot, wherein the first plot is modified such that an indicia gets modified when a cell, subclonotype, or clonotype corresponding to the indicia does pass a criteria of the selected filter. Moreover, the indicia can have a first color, and the indicia can change to a second color if modified according to the criteria of the selected filter.

Further, in accordance with various embodiments of a system, the filter selection received can include a selected feature, and at least one tunable parameter of the selected feature. Also, the filter selection received can include a selected feature, and more than one tunable parameter of the selected feature.

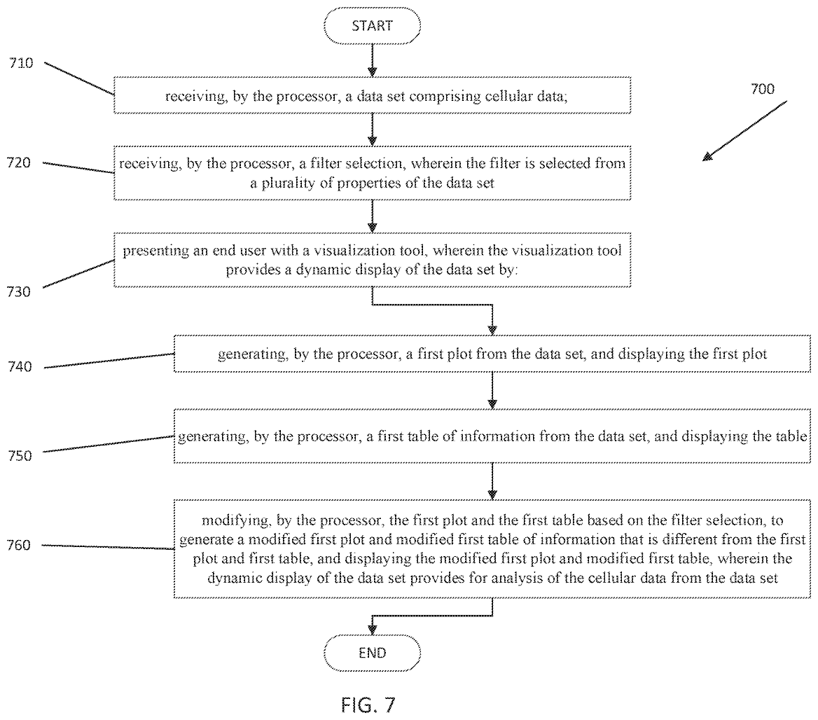

In accordance with various embodiments, FIG. 7 illustrates a non-limiting example of a computer-readable storage medium encoded with a series of instructions 700, executable by a processor, for a visualization tool for cellular data. The instructions 700 can include a first operation 710, comprising receiving, by the processor, a data set comprising cellular data

The instructions can include a second operation 720, comprising receiving, by the processor, a filter selection, wherein the filter is selected from a plurality of properties of the data set.

The instructions can include a third operation 730, comprising presenting an end user with a visualization tool. The visualization tool can provide a dynamic display of the data set by, for example, fourth operation 740, fifth operation 750 and sixth operation 760.

For the fourth operation 740, the visualization tool can provide a dynamic display of the data set by generating, by the processor, a first plot from the data set, and displaying the first plot.

For the fifth operation 750, the visualization tool can provide a dynamic display of the data set by generating, by the processor, a first table of information from the data set, and displaying the table.

For the sixth operation 760, the visualization tool can provide a dynamic display of the data set by modifying, by the processor, the first plot and the first table based on the filter selection, to generate a modified first plot and modified first table of information that is different from the first plot and first table, and displaying the modified first plot and modified first table. The dynamic display of the data set can provide for analysis of the cellular data from the data set. Further detailed discussion regarding the various features of the dynamic display will be provided below.

As stated above, it should be understood that the processes discussed herein are not subject to the particular order of steps described. As an example of that, and in accordance with various embodiments, another non-limiting example of a computer implemented method for a visualization tool for cellular data is provided. The method can comprise receiving, by the processor, a data set comprising cellular data (see, e.g., operation 710). The method can further comprise presenting an end user with a visualization tool (see, e.g., operation 730). The visualization tool can provide a dynamic display of the data set by generating, by the processor, a first plot from the data set (see, e.g., operation 740), and displaying the first plot. The visualization tool can further provide a dynamic display of the data set by generating, by the processor, a first table of information from the data set, and displaying the table (see, e.g., operation 750). The method can further comprise receiving, by the processor, a filter selection, wherein the filter is selected from a plurality of properties of the data set (see, e.g., operation 720). The method can further comprise modifying, by the processor, the first plot and the first table based on the filter selection, to generate a modified first plot and modified first table of information that is different from the first plot and first table, and displaying the modified first plot and modified first table (see, e.g., operation 760). The dynamic display of the data set can provide for analysis of the cellular data from the data set.

Further, in accordance with various embodiments of a computer-readable storage medium encoded with instructions, executable by a processor, for a visualization tool for cellular data, the instructions 700 can further comprise receiving, by the processor, a plurality of filter selections, wherein each filter is selected from the group consisting of UMI Counts/antigen, binding specificity, barcode, number of barcodes representing a clonotype, CDR amino acid sequence (e.g., CDR3 amino acid sequence), CDR bases (e.g., CDR3 bases), gene name, isotype, cluster, amino acid % mutation, nucleotide % mutation, iNKT/MAIT, and combinations thereof.

In addition, in accordance with various embodiments of a computer-readable storage medium encoded with instructions, executable by a processor, for a visualization tool for cellular data, the instructions can further comprise receiving, by the processor, a plurality of filter selections, wherein each filter is selected from the group consisting of UMI Counts/antigen, binding specificity, barcode, number of barcodes representing a clonotype, isotype, cluster, amino acid % mutation, nucleotide % mutation, iNKT/MAIT, and combinations thereof.

Further, in accordance with various embodiments of a computer-readable storage medium encoded with instructions, executable by a processor, for a visualization tool for cellular data, the cellular data can include T-Cell data. The cellular data can include B-Cell data.

Additionally, in accordance with various embodiments of a computer-readable storage medium encoded with instructions, executable by a processor, for a visualization tool for cellular data, at least one of the first plot and modified first plot can comprise a clonotype distribution plot.

As well, in accordance with various embodiments of a computer-readable storage medium encoded with instructions, executable by a processor, for a visualization tool for cellular data, at least one of the first table and modified first table can comprise genetic information for identified clonotypes or cells belonging to a clonotype presented in the at least one of the first or modified first plots. The genetic information can be selected from the group consisting or V-gene, D-gene, J-gene, CDR sequence, and combinations thereof.

In addition, in accordance with various embodiments of a computer-readable storage medium encoded with instructions, executable by a processor, for a visualization tool for cellular data, the filter can be selected from multiple properties of VDJ sequences for heavy and light chains. Moreover, the filter can be selected from the group consisting of UMI Counts/antigen, antigen specificity, barcode, number of barcodes representing a clonotype, CDR amino acid sequence (e.g., CDR3 amino acid sequence), CDR bases (e.g., CDR3 bases), gene name, isotype, cluster, amino acid % mutation, nucleotide % mutation, iNKT/MAIT information, and combinations thereof. Even further, the filter can be selected from the group consisting of UMI Counts/antigen, antigen specificity, barcode, number of barcodes representing a clonotype, isotype, cluster, amino acid % mutation, nucleotide % mutation, INKT/MAIT information, and combinations thereof.

Further, in accordance with various embodiments of a computer-readable storage medium encoded with instructions, executable by a processor, for a visualization tool for cellular data, the first plot can comprise a plurality of indicia, wherein each indicia represents a cell. The instructions can further comprise modifying, by the processor, the first plot based on the filter selection, to generate the modified first plot that is different from the first plot, and displaying the modified first plot, wherein the first plot is modified such that an indicia gets modified when a cell, subclonotype, or clonotype corresponding to the indicia does not pass a criteria of the selected filter.

Also, in accordance with various embodiments of a computer-readable storage medium encoded with instructions, executable by a processor, for a visualization tool for cellular data, the instructions can further comprise modifying, by the processor, the first plot based on the filter selection, to generate the modified first plot that is different from the first plot, and displaying the modified first plot, wherein the first plot is modified such that an indicia gets modified when a cell, subclonotype, or clonotype corresponding to the indicia does pass a criteria of the selected filter.

Further, in accordance with various embodiments of a computer-readable storage medium encoded with instructions, executable by a processor, for a visualization tool for cellular data, the indicia can have a first color, and the indicia can be changed to a second color if modified according to the criteria of the selected filter.

Further, in accordance with various embodiments of a computer-readable storage medium encoded with instructions, executable by a processor, for a visualization tool for cellular data, the filter selection received can include a selected feature, and at least one tunable parameter of the selected feature. Also, the filter selection received can include a selected feature, and more than one tunable parameter of the selected feature.

In accordance with various embodiments, a computer implemented method for a visualization tool for cellular data is disclosed. The method can share steps similar to, for example, those operations 710-760 illustrated in FIG. 7. The method can comprise receiving, by the processor, a data set comprising cellular data (see, e.g., operation 710). The method can comprise receiving, by the processor, a filter selection, wherein the filter is selected from a plurality of properties of the data set (see, e.g., operation 720). The method can comprise presenting an end user with a visualization tool (see, e.g., operation 730). The visualization tool can provide a dynamic display of the data set by generating, by the processor, a first plot from the data set, and displaying the first plot (see, e.g., operation 740), generating, by the processor, a first table of information from the data set, and displaying the table (see, e.g., operation 750), and modifying, by the processor, the first plot and the first table based on the filter selection, to generate a modified first plot and modified first table of information that is different from the first plot and first table, and displaying the modified first plot and modified first table (see, e.g., operation 760). The dynamic display of the data set can provide for analysis of the cellular data from the data set. Further detailed discussion regarding the various features of the dynamic display will be provided below.

Further, in accordance with various embodiments for a computer implemented method of a visualization tool for cellular data, the method can further comprise receiving, by the processor, a plurality of filter selections, wherein each filter is selected from the group consisting of UMI Counts/antigen, binding specificity, barcode, number of barcodes representing a clonotype, CDR amino acid sequence (e.g., CDR3 amino acid sequence), CDR bases (e.g., CDR3 bases), gene name, isotype, cluster, amino acid % mutation, nucleotide % mutation, iNKT/MAIT, and combinations thereof.

In addition, in accordance with various embodiments for a computer implemented method of a visualization tool for cellular data, the method can further comprise receiving, by the processor, a plurality of filter selections, wherein each filter is selected from the group consisting of UMI Counts/antigen, binding specificity, barcode, number of barcodes representing a clonotype, isotype, cluster, amino acid % mutation, nucleotide % mutation, iNKT/MAIT, and combinations thereof.

Further, in accordance with various embodiments of a computer implemented method of a visualization tool for cellular data, the cellular data can include T-Cell data. The cellular data can include B-Cell data.

Additionally, in accordance with various embodiments of a computer implemented method of a visualization tool for cellular data, at least one of the first plot and modified first plot can comprise a clonotype distribution plot.

As well, in accordance with various embodiments of a computer implemented method of a visualization tool for cellular data, at least one of the first table and modified first table can comprise genetic information for identified clonotypes or cells belonging to a clonotype presented in the at least one of the first or modified first plots. The genetic information can be selected from the group consisting or V-gene, D-gene, J-gene, CDR sequence, and combinations thereof.

In addition, in accordance with various embodiments of a computer implemented method of a visualization tool for cellular data, the filter can be selected from multiple properties of VDJ sequences for heavy and light chains. Moreover, the filter can be selected from the group consisting of UMI Counts/antigen, antigen specificity, barcode, number of barcodes representing a clonotype, CDR amino acid sequence (e.g., CDR3 amino acid sequence), CDR bases (e.g., CDR3 bases), gene name, isotype, cluster, amino acid % mutation, nucleotide % mutation, iNKT/MAIT information, and combinations thereof. Even further, the filter can be selected from the group consisting of UMI Counts/antigen, antigen specificity, barcode, number of barcodes representing a clonotype, isotype, cluster, amino acid % mutation, nucleotide % mutation, iNKT/MAIT information, and combinations thereof.

Further, in accordance with various embodiments of a computer implemented method of a visualization tool for cellular data, the first plot can comprise a plurality of indicia, wherein each indicia represents a cell. The method can further comprise modifying, by the processor, the first plot based on the filter selection, to generate the modified first plot that is different from the first plot, and displaying the modified first plot, wherein the first plot is modified such that an indicia gets modified when a cell, subclonotype, or clonotype corresponding to the indicia does not pass a criteria of the selected filter.

Also, in in accordance with various embodiments of a computer implemented method of a visualization tool for cellular data, the method can further comprise modifying, by the processor, the first plot based on the filter selection, to generate the modified first plot that is different from the first plot, and displaying the modified first plot, wherein the first plot is modified such that an indicia gets modified when a cell, subclonotype, or clonotype corresponding to the indicia does pass a criteria of the selected filter.

Further, in in accordance with various embodiments of a computer implemented method of a visualization tool for cellular data, the indicia can have a first color, and the indicia can be changed to a second color if modified according to the criteria of the selected filter.

Further, in accordance with various embodiments of a computer implemented method for a visualization tool for cellular data, the filter selection received can include a selected feature, and at least one tunable parameter of the selected feature. Also, the filter selection received can include a selected feature, and more than one tunable parameter of the selected feature.

In accordance with various embodiments, a system is provided. The system can comprise a processor and a memory in communication with the processor. The memory can store a series of instructions, steps or operations, similar to the instructions 700 (and associated operations 710 to 760) illustrated in FIG. 7. The memory can store instructions for receiving, by the processor, a data set comprising cellular data (see, e.g., operation 710). The memory can store instructions for receiving, by the processor, a filter selection, wherein the filter is selected from a plurality of properties of the data set (see, e.g., operation 720). The memory can store instructions for presenting an end user with a visualization tool (see, e.g., operation 730). The visualization tool can provide a dynamic display of the data set by generating, by the processor, a first plot from the data set, and displaying the first plot (see, e.g., operation 740), generating, by the processor, a first table of information from the data set, and displaying the table (see, e.g., operation 750), and modifying, by the processor, the first plot and the first table based on the filter selection, to generate a modified first plot and modified first table of information that is different from the first plot and first table, and displaying the modified first plot and modified first table (see, e.g., operation 760). The dynamic display of the data set can provide for analysis of the cellular data from the data set. Further detailed discussion regarding the various features of the dynamic display will be provided below.

Further, in accordance with various embodiments of a system, the system can further store instructions for receiving, by the processor, a plurality of filter selections, wherein each filter is selected from the group consisting of UMI Counts/antigen, binding specificity, barcode, number of barcodes representing a clonotype, CDR amino acid sequence (e.g., CDR3 amino acid sequence), CDR bases (e.g., CDR3 bases), gene name, isotype, cluster, amino acid % mutation, nucleotide % mutation, iNKT/MAIT, and combinations thereof.

Further, in accordance with various embodiments of a system, the system can further store instructions for receiving, by the processor, a plurality of filter selections, wherein each filter is selected from the group consisting of UMI Counts/antigen, binding specificity, barcode, number of barcodes representing a clonotype, CDR amino acid sequence (e.g., CDR3 amino acid sequence), CDR bases (e.g., CDR3 bases), gene name, isotype, cluster, amino acid % mutation, nucleotide % mutation, iNKT/MAIT, and combinations thereof.

Further, in accordance with various embodiments of a system, the cellular data can include T-Cell data. The cellular data can include B-Cell data.

Additionally, in accordance with various embodiments of a system, at least one of the first plot and modified first plot can comprise a clonotype distribution plot.

As well, in accordance with various embodiments of a system, at least one of the first table and modified first table can comprise genetic information for identified clonotypes or cells belonging to a clonotype presented in the at least one of the first or modified first plots. The genetic information can be selected from the group consisting or V-gene, D-gene, J-gene, CDR sequence, and combinations thereof.

In addition, in accordance with various embodiments of a system, the filter can be selected from multiple properties of VDJ sequences for heavy and light chains. Moreover, the filter can be selected from the group consisting of UMI Counts/antigen, antigen specificity, barcode, number of barcodes representing a clonotype, CDR amino acid sequence (e.g., CDR3 amino acid sequence), CDR bases (e.g., CDR3 bases), gene name, isotype, cluster, amino acid % mutation, nucleotide % mutation, iNKT/MAIT information, and combinations thereof. Even further, the filter can be selected from the group consisting of UMI Counts/antigen, antigen specificity, barcode, number of barcodes representing a clonotype, isotype, cluster, amino acid % mutation, nucleotide % mutation, iNKT/MAIT information, and combinations thereof.

Further, in accordance with various embodiments of a system, the first plot can comprise a plurality of indicia, wherein each indicia represents a cell. The system can further store instructions for modifying, by the processor, the first plot based on the filter selection, to generate the modified first plot that is different from the first plot, and displaying the modified first plot, wherein the first plot is modified such that an indicia gets modified when a cell, subclonotype, or clonotype corresponding to the indicia does not pass a criteria of the selected filter.

Also, in accordance with various embodiments of a system, the system can further store instructions for modifying, by the processor, the first plot based on the filter selection, to generate the modified first plot that is different from the first plot, and displaying the modified first plot, wherein the first plot is modified such that an indicia gets modified when a cell, subclonotype, or clonotype corresponding to the indicia does pass a criteria of the selected filter.

Further, in accordance with various embodiments of a system, the indicia can have a first color, and the indicia can be changed to a second color if modified according to the criteria of the selected filter.

Further, in accordance with various embodiments of a system, the filter selection received can include a selected feature, and at least one tunable parameter of the selected feature. Also, the filter selection received can include a selected feature, and more than one tunable parameter of the selected feature.

The dynamic real-time updating of both the plot and table (as described for example in FIG. 7) serves many advantages. The table gives details about, for example, VDJ and CDR3 information about a clonotype while a clonotype distribution plot (CD) shows a visual representation of the clonotype. Dynamically updating these together allows users to understand the VDJ properties of clonotypes that have cells that passed the filter to test/create hypothesis more quickly. Additional details about VDJ properties can be available by clicking on the CD plot to get to the sequence view. However, the ability to view both the distribution plot and the clonotype table allows a zoomed out view of the entire experiment results. A CD plot also allows one to visualize the distribution of UMI counts/antigen across all filtered cells in the experiment but without understanding the sequence properties. Together, the plot and table complement each other by providing different information types that allow for a robust overview of the experiment.

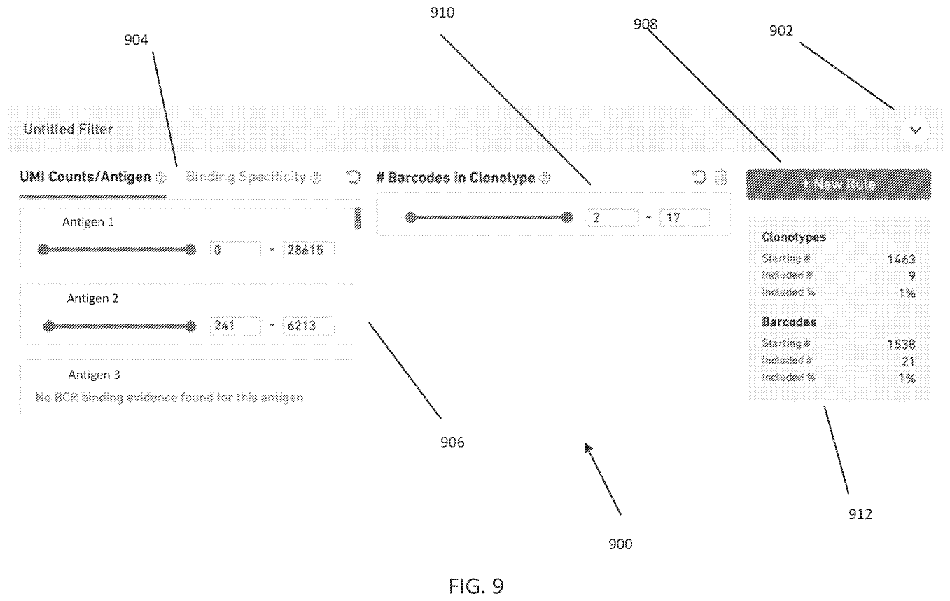

Referring to FIG. 8, a illustrates an example output display 802 of a visualization tool 800 for cellular data, in accordance with various embodiments. The display 802 can include, for example, a first plot 804, a first table 806, a filter panel 808 (discussed in detail with reference to FIG. 9) and a filter control field 810. As illustrated, filter panel 808 can be expanded via disclosure widget 812.

In this example display 802, first plot 804 is a clonotype distribution plot, which is linked with first table 806 and filter panel 808 (and associated field 810). Clonotype distribution plots, such as first plot 804, can provide an overview of all the cells (e.g., T and B cells) in the experiment grouped based on the clonotypes they belong to. In this example, each dot in the clonotype distribution plot can represent a cell.

First table 806 (linked with first plot 804) is, in this example, the clonotype list that displays key information (including, for example, V genes, D genes, J genes, CDR3 and count) about the clonotypes corresponding to the cells in the clonotype distribution.

Filter control field 810 can include a filter naming line 814 for naming (e.g., via user input) the filter (or series of filters) generated through interaction with filter panel 808. Line 814 can include a drop down button 816 to provide further options to user including, for example, options for editing an existing filter name, deleting the filter entirely, as well as downloading clonotypes onto the visualization tool 800 (e.g., for further analysis).

Display 802 can also include a display preference panel 818, which can provide options for how first plot 804 can be displayed. As stated above, in this example display 802, first plot 804 is a clonotype distribution plot. When displaying clonotypes, being able to, for example, color clonotypes by different metrics (including, e.g., binding and V(D)J properties) can be very useful generally. Additional information can be overlaid about cells and clonotypes in a clonotype distribution plot by coloring the cells based on the different metrics. For example, understanding key V(D)J metrics for both cells can allow users to detect common V(D)J metrics of clonally expanded cells (if any). Moreover, such a feature can be useful even when working with an single cell immune profiling solution without antigen binding. From a TCR and antibody discovery point of view, for example, this feature allows users to obtain a birds eye view of binding specificity metrics across the experiment.

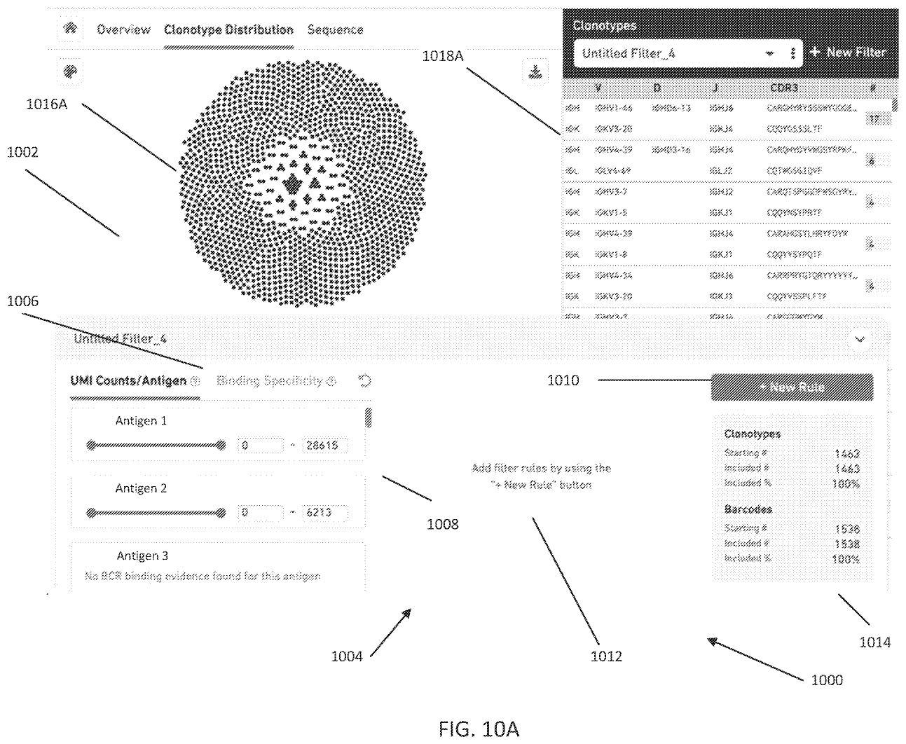

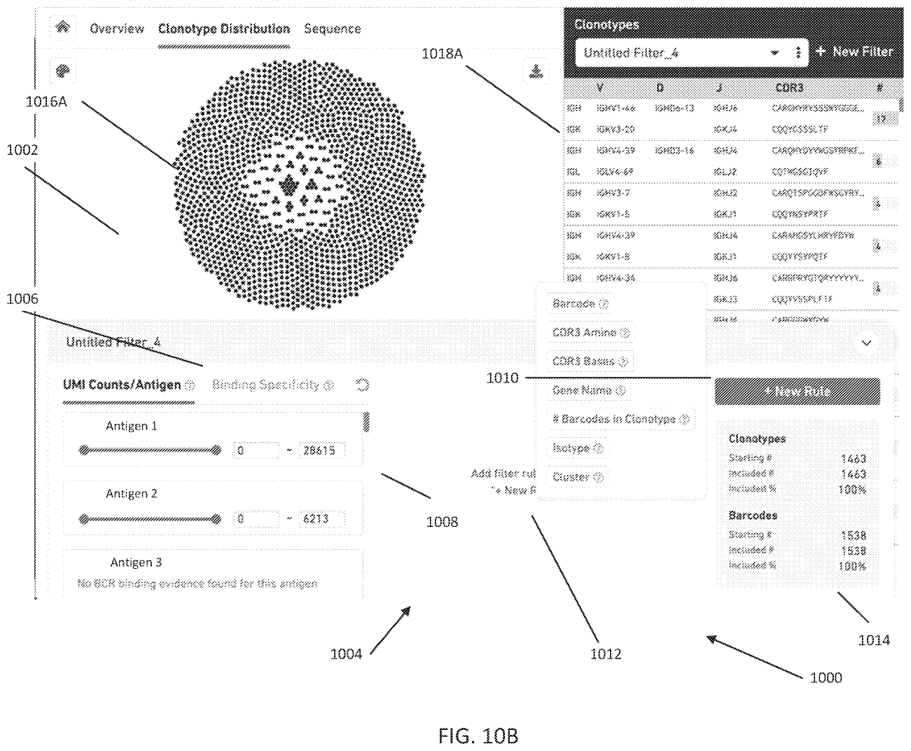

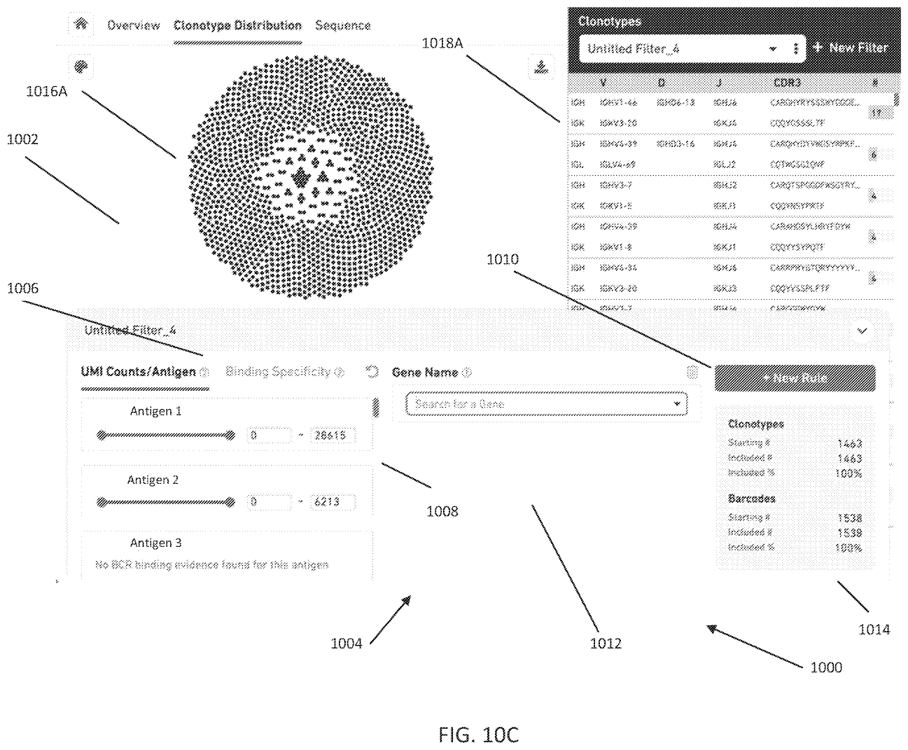

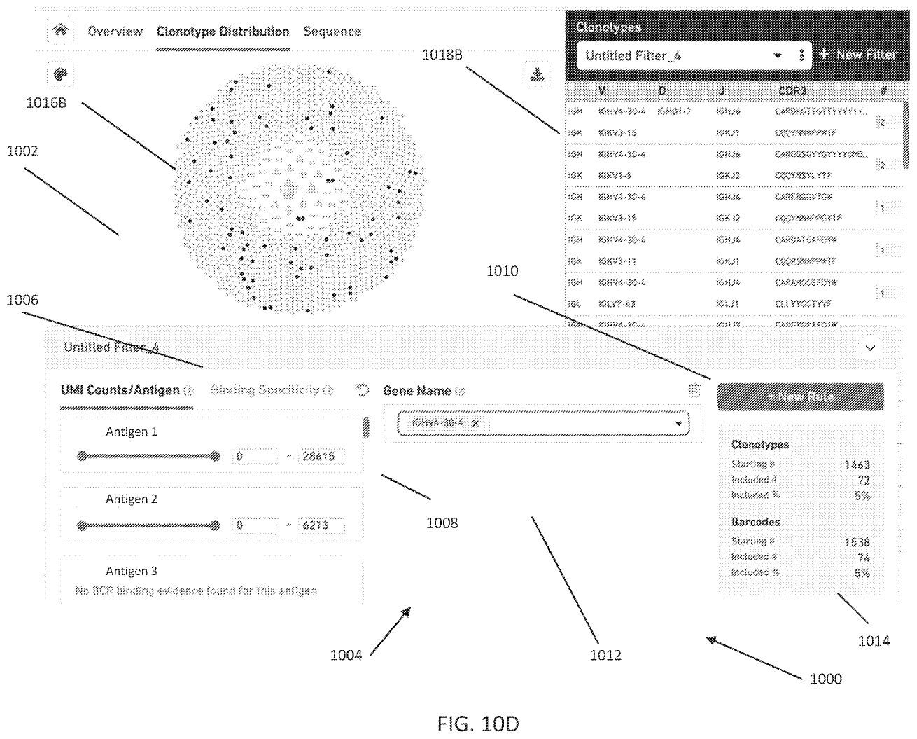

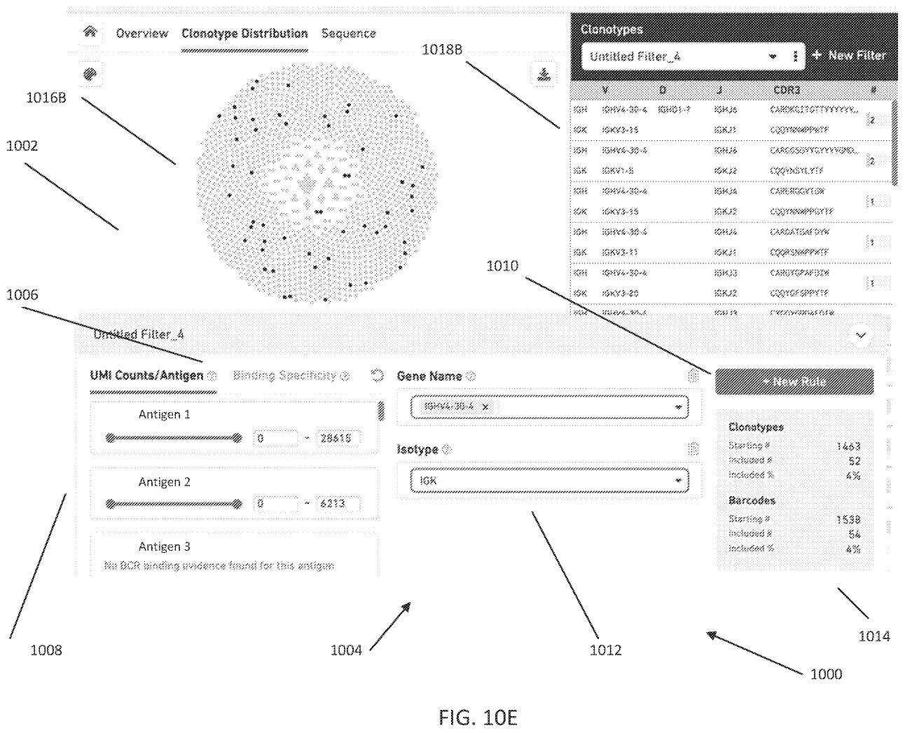

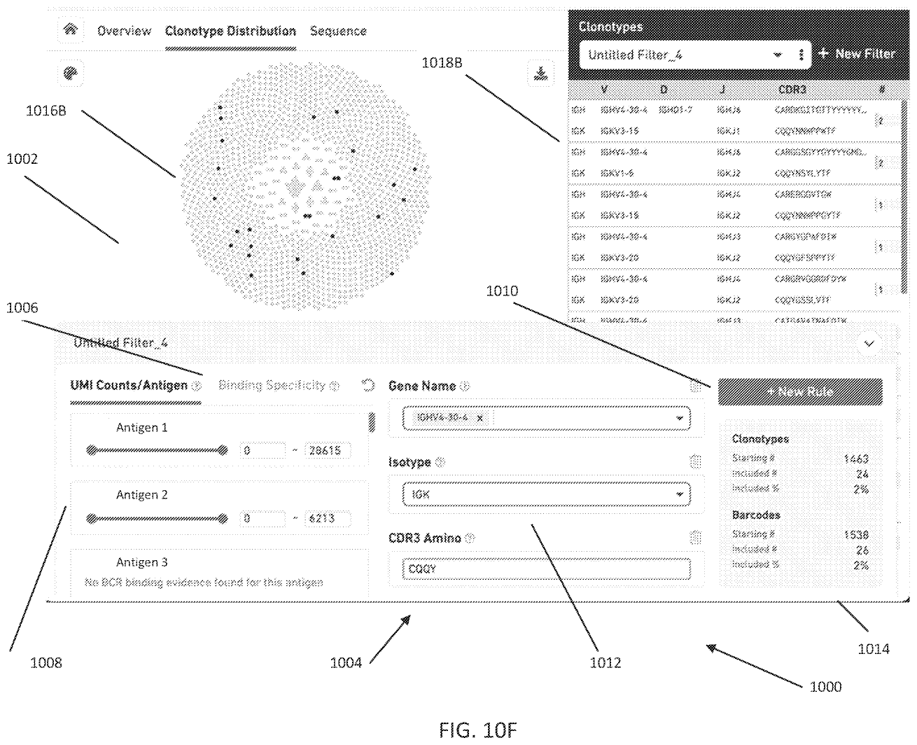

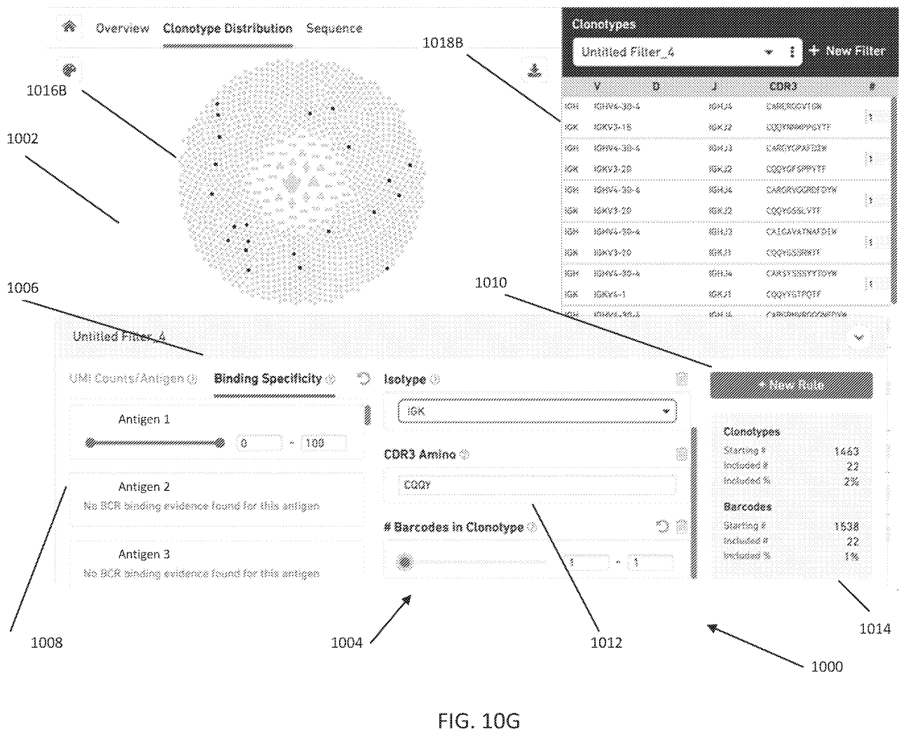

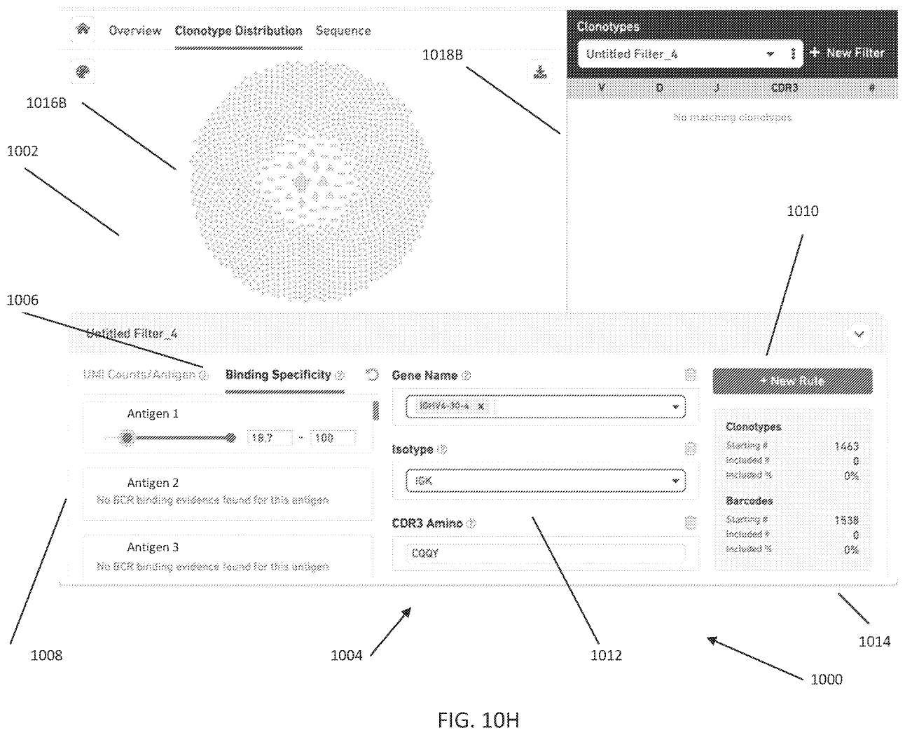

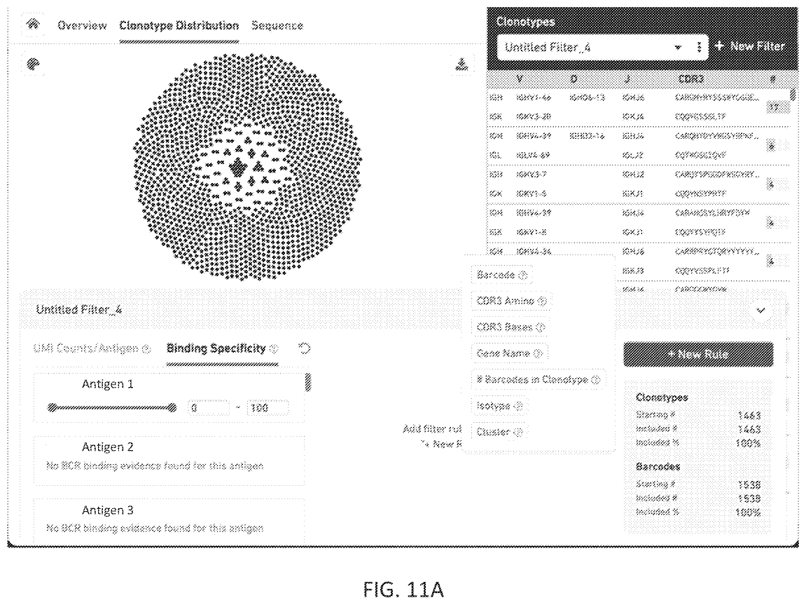

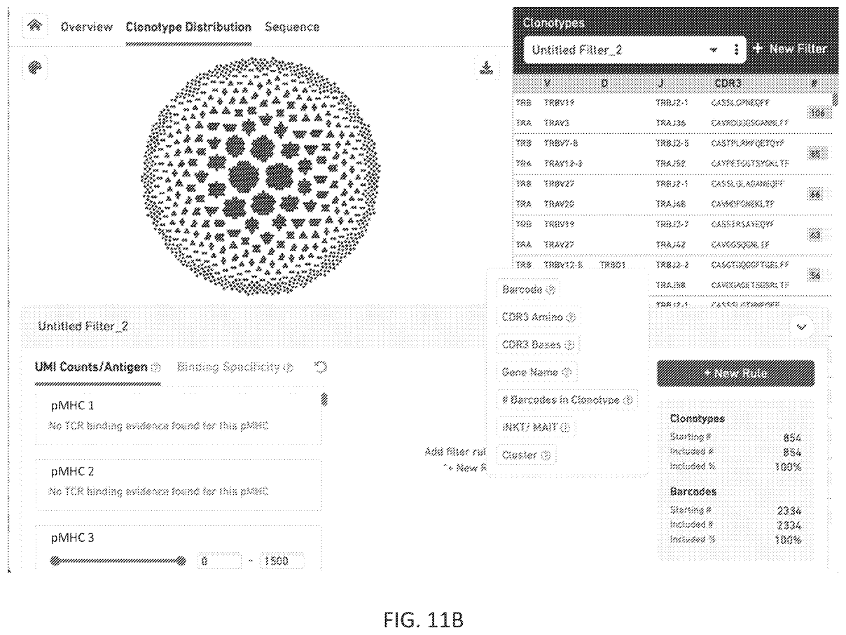

Referring now to FIG. 9, an example of a filter panel 900 is illustrated, in accordance with various embodiments. Filter panel 900 shows the display of the panel 900 after a disclosure widget 902 is engaged to expand the panel from the minimized view of panel 808 shown in FIG. 8. Note that a display including expanded filter panel is illustrated in subsequent FIGS. 10A-11B, discussed below. Note also that disclosure widget 812 of FIG. 8 is shown pointing directionally upward, indicating, in that example, that the panel will expand if the widget is engaged. On the other hand, FIG. 9, illustrating an already expanded panel 900, shows widget 902 pointing directionally downward, indicating, in that example, that the panel will collapse if the widget 902 is engaged.

Panel 900 of FIG. 9 can also include, without limitation, a filter pane 904, filter pane parameters 906, filter button 908, selected filter pane 910, and output space 912.

Filter pane 904 can include one or more filters (preset in this instance), with accompanying parameters 906, that can be selected and/or modified per, for example, user input. In the example pane 904 of FIG. 9, both a UMI Counts/Antigen filter and Binding Specificity filter are provided. As shown, a user can toggle between both provided filters, with the UMI Counts/Antigen filter being active in FIG. 9. For any of the associated filter pane parameters 906, a tunable feature may be provided to allow modification of the parameter beyond simply data entry (which is an available feature as well). As shown, the tunable feature is a slider bar for parameters TotalSeq-C0951 PE and TotalSeq-C0952 PE.

Filter button 908 can provide access, for example, to a list of available filters for selection. These filters will be discussed in detail below. The list can be provided in various forms, not limited to a popover, pop-up window, or palette window. When a filter is selected from this list, the filter can be provided for viewing on the selected filter pane 910. Moreover, once selected and viewable, the parameters of the selected filter can be modified much like the filter pane parameters 906 of filter pane 904. For example, a tunable feature may be provided to allow modification of the parameter beyond simply data entry (which is an available feature as well). As shown, the tunable feature is a slider bar for the number of barcodes in the particular clonotype being analyzed.

There are many filters available for use within the filter panel. A non-limiting list, provided as examples only, can include gene name (e.g., list of V/D/J genes), isotype (e.g., list of B cell isotypes), CDR3 Amino (e.g., CDR3 sequence in an amino acids format), CDR3 Bases (e.g., CDR3 sequence in a nucleotide bases format), iNKT/MAIT (e.g., evidence if cell type is iNKT/MAIT/Both/None), binding specificity (e.g., measure of how specific binding is to the target antigen), UMI counts/antigen (e.g., number of UMIs detected), barcode (e.g., cell identifier that maps sequencing reads to individual cells), and # barcodes in the clonotype (e.g., measure of how large a clonotype is).

For B-cells, # barcodes in the clonotype is a measure of how many cells originated from the same progenitor B-cell. For T-cell, # barcodes in the clonotype is a measure of similarity of VDJ sequence across cells.

Another selectable filter is a cluster filter. For example, when the cells of a cellular data set are previously annotated using, for example, gene expression or reporter oligonucleotide data, and those files are imported onto the visualization tool, users can filter cells based on clusters they previously annotated.

Output space 912 of panel 900 displays information relevant to the associated cellular analysis, and can be modified in real time in response to filter selection and/or modification of parameters associated with the filter selection. In the example space 912 of FIG. 9, output space 912 displays the total starting number of clonotypes, the included number of clonotypes after filter selection, and that associated percentage versus the total number. Space 912 also displays the total starting number of barcodes, the included number of barcodes after filter selection, and that associated percentage versus the total number.

The following FIGS. 10A-10H will illustrate various example output displays (see 802 of FIG. 8) of a visualization tool (see 800 of FIG. 8) for set of cellular data, illustrating the various features of the tool along with the real time, dynamic modifications to the outputs per filter selections. Though many of the features of previously discussed output displays will be included in these figures, discussion will largely be limited to those features changing from figure to figure.

FIG. 10A illustrates an example output display 1002 of a visualization tool 1000 for cellular data, in accordance with various embodiments. Display 1002 includes a filter panel 1004 having a filter pane 1006, filter pane parameters 1008, filter button 1010, selected filter pane 1012, and output space 1014. In this figure, filter panel 1004 has been expanded for use, while neither filter on filter pane 1006 has been modified, nor has a new filter been selected via filter button 1010. As such, no filter is listed in the selected filter pane 1012, and no clonotypes or barcodes have been excluded (or filtered out), leaving inclusion percentages at 100% on output space 1014. Output display also includes first plot 1016A and first table 1018A.

FIG. 10B illustrates another example output display 1002 of visualization tool 1000 for cellular data, in accordance with various embodiments. Versus FIG. 10A, filter button 1010 has been engaged, revealing a pop-up window with a list of selectable filters (Barcode, CDR3 Amino, CDR3 Bases, Gene name, #Barcodes in Clonotype, Isotype and Cluster). Similar to FIG. 10A, neither filter on filter pane 1006 has been modified, nor has a new filter been selected via filter button 1010. As such, no filter is listed in the selected filter pane 1012, and no clonotypes or barcodes have been excluded (or filtered out), leaving inclusion percentages at 100% on output space 1014.

FIG. 10C illustrates another example output display 1002 of visualization tool 1000 for cellular data, in accordance with various embodiments. In this figure, Gene name has been selected as a filter. As such, this selection is reflected in selected filter pane 1012. However, since no gene has been inputted at this juncture, no actual filtering has occurred, leaving inclusion percentages still at 100% on output space 1014.

FIG. 10D illustrates another example output display 1002 of visualization tool 1000 for cellular data, in accordance with various embodiments. In this figure, Gene name has been selected as a filter and Gene IGHV4-30-4 has been selected. As such, this selection is reflected in selected filter pane 1012. Moreover, with the filter selection, first plot 1016A and first table 1018A have been dynamically modified to generate a second plot 1016B and second table 1018B. Moreover, output space 1014 has also been modified in real time to reflect new statistics for included clonotypes and barcodes, and their associated percentages relative to the total starting number of clonotypes and barcodes.

FIG. 10E illustrates another example output display 1002 of visualization tool 1000 for cellular data, in accordance with various embodiments. In this figure, Gene name has been selected as a filter (Gene IGHV4-30-4 having been selected) and Isotype has been selected as a filter (IGK having been selected). As such, these selections are reflected in selected filter pane 1012. Moreover, with the filter selection, first plot 1016A and first table 1018A have been dynamically modified to generate a second plot 1016B and second table 1018B, different from that illustrated in FIG. 10D. Moreover, output space 1014 has also been modified in real time to reflect new statistics for included clonotypes and barcodes, and their associated percentages relative to the total starting number of clonotypes and barcodes.

FIG. 10F illustrates another example output display 1002 of visualization tool 1000 for cellular data, in accordance with various embodiments. In this figure, Gene name has been selected as a filter (Gene IGHV4-30-4 having been selected), Isotype has been selected as a filter (IGK having been selected), and CDR3 Amino has been selected as a filter (CQQY having been selected). As such, these selections are reflected in selected filter pane 1012. Moreover, with the filter selection, first plot 1016A and first table 1018A have been dynamically modified to generate a second plot 1016B and second table 1018B, different from that illustrated in FIGS. 10D and 10E. Moreover, output space 1014 has also been modified in real time to reflect new statistics for included clonotypes and barcodes, and their associated percentages relative to the total starting number of clonotypes and barcodes.

FIG. 10G illustrates another example output display 1002 of visualization tool 1000 for cellular data, in accordance with various embodiments. In this figure, Gene name has been selected as a filter (Gene IGHV4-30-4 having been selected), Isotype has been selected as a filter (IGK having been selected), CDR3 Amino has been selected as a filter (CQQY having been selected), and # of Barcodes in Clonotype has been selected as a filter (one barcode haven been selected via movement of the slider bar entirely to the left). As such, these selections are reflected in selected filter pane 1012. Note that with four filters now being selected, and given space constraints, the filters can be moved by scrolling up and down within the selected filter pain 1012. Moreover, with the filter selection, first plot 1016A and first table 1018A have been dynamically modified to generate a second plot 1016B and second table 1018B, different from that illustrated in FIGS. 10D, 10E, and 10F. Moreover, output space 1014 has also been modified in real time to reflect new statistics for included clonotypes and barcodes, and their associated percentages relative to the total starting number of clonotypes and barcodes. Note also that, for filter pane 1006, user has toggled from UMI Counts/Antigen to Binding Specificity, though no modification of the Binding Specificity has occurred.

FIG. 10H illustrates another example output display 1002 of visualization tool 1000 for cellular data, in accordance with various embodiments. In this figure, Gene name has been selected as a filter (Gene IGHV4-30-4 having been selected), Isotype has been selected as a filter (IGK having been selected), CDR3 Amino has been selected as a filter (CQQY having been selected), and # of Barcodes in Clonotype has been selected as a filter (one barcode haven been selected via movement of the slider bar entirely to the left). Moreover, for filter pane 1006, user has toggled from UMI Counts/Antigen to Binding Specificity and modified TotalSeq-C0951 PE via slider bar. As such, these selections are reflected in selected filter pane 1012 and filter pane 1006. Note that with four filters now being selected on the filter pane 1012, and given space constraints, the filters can be moved by scrolling up and down within the selected filter pain 1012. Moreover, with the filter selection, first plot 1016A and first table 1018A have been dynamically modified to generate a second plot 1016B and second table 1018B, different from that illustrated in FIGS. 10D, 10E, 10F and 10G. Moreover, output space 1014 has also been modified in real time to reflect new statistics for included clonotypes and barcodes, and their associated percentages relative to the total starting number of clonotypes and barcodes. In this case, with all the above filters selected, zero clonotypes and zero barcodes are included.

FIGS. 11A and B illustrate example output displays of a visualization tool for cellular data, in accordance with various embodiments. In these examples, the output display for FIG. 11A is for B cell analysis and the output display for FIG. 11B is for T cell analysis. Based on the analysis being conducted, the types of filters available can be adapted accordingly. For example, one difference to note is the presence of a Isotype filter in the display of FIG. 11A (for B cell analysis) and the presence of a iNKT/MAIT filter in the display of FIG. 11B (for T cell analysis).

It should be noted that, in accordance with various embodiments herein, that information about individual cells can be obtained through interaction with the visualization tool. For example, a user can roll over or select (via any selection tool including, for example, mouse click) a specific cell of interest on the plot. The tool can respond by revealing, for example, sequence information related to that cell. This reveal can occur in many ways including, for example, a pop-up window.

### Example Applications

The following section provides examples applications of how the provided systems can be used for generating specific insights that are valuable to researchers (e.g., immunology researchers).

### Identifying Expanded or Rare Clonotypes

In this example, a user may utilize the provided systems to identify expanded or rare clonotypes. To do so, a user selects to color the clonotype distribution plot based on antigen specificity score and find which clonotype is associated with the target antigen of interest. For example, a sample from a COVID patient may show expanded clonotypes with a high antigen specificity score for COVID spike proteins. The user then filters the data by antigen specificity of the target antigen. The user also filters the data by a number of barcodes per clonotype. If the user is looking for expanded clonotypes, the user may set the lower limit for the number of barcode per clonotype to higher value. The purpose of studying expanded clonotypes is to characterize all potential or new antigen relationships with that B or T cell receptor of interest (e.g., immuno therapy or discovery work to see if a known antibody binds to a different antigen). If the user is looking for rare clonotypes, the user may set the upper limit for the number of barcode per clonotype to a lower value. For example, when studying a COVID patient with antibodies against COVID, a researcher may investigate whether the patient has a rare antibody that allows the patient to survive at a faster rate before the clonotype starts to expand.

### Antibody or T-Cell Receptor (TCR) Discovery

In this example, a user may utilize the provided systems to discover one or more antibodies. To do so, a user filters the data by antigen specificity or UMI count per antigen, and filters for cell receptors that have a high score for the target antigen of interest. In some instances, the user may also utilize other filters to identify cell receptors based on VDJ gene or CDR sequences that the user is interested in. The user then has a few options to aid antibody discovery. The user can export the clonotype table of information. The user can view exact subclonotypes in the sequence view and export the sequences. The user can star sequences of interest, and export the starred sequences. The user can then import the exported sequences into other applications the user is using to perform cloning to propagate and perform experiments on specific receptor sequences the user identified through the provided systems.

### T-Cell or B-Cell Subtype-Based Antibody Discovery

In this example, a user may utilize the provided systems to discover one or more T-cell or B-cell subtype-based antigen binding molecules (e.g., B-cell receptors (BCRs), antibodies, TCRs, or antigen binding fragments thereof). To do so, a user annotates a data file based on gene expression to identify memory T-cells (or B-cells). The user then loads the annotated data file into the provided system and selects the memory T-cell (or B-cell) category to filter the clonotypes. The user then filters the data based on antigen specificity score to identify memory T-cells (or B-cells) that have high scores for a target antigen. Finally, the user exports the filtered clonotype table or specific sequences for subsequent experiments.

### Gene Expression Profile of Cells Expressing Receptors of Interest

In this example, a user may utilize the provided systems to investigate a gene expression profile of cells expressing receptors of interest. To do so, a user performs filtering on the data, e.g., selecting expanded clonotypes that have a high antigen specificity score to a target antigen. The user then exports the barcodes of the filtered data and imports the exported file into another application the user is using to explore gene expression differences in the cells expressing receptors of interest compared to the rest of the cells.

### Computer-Implemented System

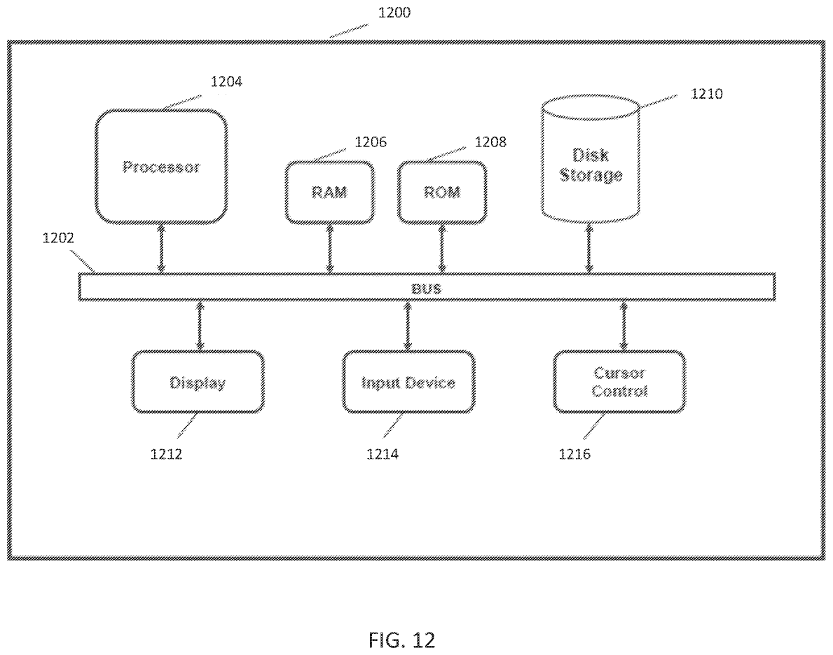

FIG. 12 is a block diagram that illustrates a computer system 1200, upon which embodiments of the present teachings may be implemented. In various embodiments of the present teachings, computer system 1200 can include a bus 1202 or other communication mechanism for communicating information, and a processor 1204 coupled with bus 1202 for processing information. In various embodiments, computer system 1200 can also include a memory, which can be a random access memory (RAM) 1206 or other dynamic storage device, coupled to bus 1202 for determining instructions to be executed by processor 1204. Memory also can be used for storing temporary variables or other intermediate information during execution of instructions to be executed by processor 1204. In various embodiments, computer system 1200 can further include a read only memory (ROM) 1208 or other static storage device coupled to bus 1202 for storing static information and instructions for processor 1204. A storage device 1210, such as a magnetic disk or optical disk, can be provided and coupled to bus 1202 for storing information and instructions.

In various embodiments, computer system 1200 can be coupled via bus 1202 to a display 1212, such as a cathode ray tube (CRT) or liquid crystal display (LCD), for displaying information to a computer user. An input device 1214, including alphanumeric and other keys, can be coupled to bus 1202 for communicating information and command selections to processor 1204. Another type of user input device is a cursor control 1216, such as a mouse, a trackball or cursor direction keys for communicating direction information and command selections to processor 1204 and for controlling cursor movement on display 1212. This input device 1214 typically has two degrees of freedom in two axes, a first axis (i.e., x) and a second axis (i.e., y), that allows the device to specify positions in a plane. However, it should be understood that input devices 1214 allowing for 3 dimensional (x, y and z) cursor movement are also contemplated herein.

Consistent with certain implementations of the present teachings, results can be provided by computer system 1200 in response to processor 1204 executing one or more sequences of one or more instructions contained in memory 1206. Such instructions can be read into memory 1206 from another computer-readable medium or computer-readable storage medium, such as storage device 1210. Execution of the sequences of instructions contained in memory 1206 can cause processor 1204 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, etc.) or “computer-readable storage medium” as used herein refers to any media that participates in providing instructions to processor 1204 for execution. Such a medium can take many forms, including but not limited to, non-volatile media, volatile media, and transmission media. Examples of non-volatile media can include, but are not limited to, optical, solid state, magnetic disks, such as storage device 1210. Examples of volatile media can include, but are not limited to, dynamic memory, such as memory 1206. Examples of transmission media can include, but are not limited to, coaxial cables, copper wire, and fiber optics, including the wires that comprise bus 1202.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 1204 of computer system 1200 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc.

It should be appreciated that the methodologies described herein flow charts, diagrams and accompanying disclosure can be implemented using computer system 1200 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Rust, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 1200, whereby processor 1204 would execute the analyses and determinations provided by these engines, subject to instructions provided by any one of, or a combination of, memory components 1206/1208/1210 and user input provided via input device 1214.

### Digital Processing Device

In various embodiments, the systems and methods described herein can include a digital processing device, or use of the same. In various embodiments, the digital processing device can includes one or more hardware central processing units (CPUs) or general-purpose graphics processing units (GPGPUs) that carry out the device's functions. In various embodiments, the digital processing device further comprises an operating system configured to perform executable instructions. In various embodiments, the digital processing device can be optionally connected a computer network. In various embodiments, the digital processing device can be optionally connected to the Internet such that it accesses the World Wide Web. In various embodiments, the digital processing device can be optionally connected to a cloud computing infrastructure. In various embodiments, the digital processing device can be optionally connected to an intranet. In various embodiments, the digital processing device can be optionally connected to a data storage device.

In accordance with various embodiments, suitable digital processing devices can include, by way of non-limiting examples, server computers, desktop computers, laptop computers, notebook computers, sub-notebook computers, netbook computers, netpad computers, handheld computers, Internet appliances, mobile smartphones, tablet computers, and personal digital assistants. Those of ordinary skill in the art will recognize that many smartphones are suitable for use in the system described herein. Those of ordinary skill in the art will also recognize that select televisions, video players, and digital music players with optional computer network connectivity are suitable for use in the system described herein. Suitable tablet computers include those with booklet, slate, and convertible configurations, known to those of ordinary skill in the art.

In various embodiments, the digital processing device includes an operating system configured to perform executable instructions. The operating system can be, for example, software, including programs and data, which manages the device's hardware and provides services for execution of applications. Those of ordinary skill in the art will recognize that suitable server operating systems include, by way of non-limiting examples, FreeBSD, OpenBSD, Net-BSD, Linux, Apple® Mac OS X Server®, Oracle® Solaris®, Windows Server®, and Novell® NetWare®. Those of ordinary skill in the art will recognize that suitable personal computer operating systems include, by way of non-limiting examples, Microsoft® Windows®, Apple® Mac OS X®, UNIX®, and UNIX-like operating systems such as GNU/Linux®. In various embodiments, the operating system is provided by cloud computing. Those of ordinary skill in the art will also recognize that suitable mobile smart phone operating systems include, by way of non-limiting examples, Nokia® Symbian® OS, Apple® iOS®, Research In Motion® Black-Berry OS®, Google® Android®, Microsoft® Windows Phone® OS, Microsoft® Windows Mobile® OS, Linux®, and Palm® WebOS®.

In various embodiments, the device includes a storage and/or memory device. The storage and/or memory device is one or more physical apparatuses used to store data or programs on a temporary or permanent basis. In various embodiments, the device is volatile memory and requires power to maintain stored information. In various embodiments, the device is non-volatile memory and retains stored information when the digital processing device is not powered. In various embodiments, the non-volatile memory comprises flash memory. In some embodiments, the non-volatile memory comprises dynamic random-access memory (DRAM). In various embodiments, the non-volatile memory comprises ferroelectric random access memory (FRAM). In various embodiments, the non-volatile memory comprises phase-change random access memory (PRAM). In various embodiments, the device is a storage device including, by way of non-limiting examples, CD-ROMs, DVDs, flash memory devices, magnetic disk drives, magnetic tapes drives, optical disk drives, and cloud computing based storage. In various embodiments, the storage and/or memory device is a combination of devices such as those disclosed herein.

In various embodiments, the digital processing device includes a display to send visual information to a user. In various embodiments, the display is a cathode ray tube (CRT). In various embodiments, the display is a liquid crystal display (LCD). In various embodiments, the display is a thin film transistor liquid crystal display (TFT-LCD). In various embodiments, the display is an organic light emitting diode (OLED) display. In various embodiments, on OLED display is a passive-matrix OLED (PMOLED) or active-matrix OLED (AMOLED) display. In various embodiments, the display is a plasma display. In various embodiments, the display is a video projector. In various embodiments, the display is a combination of devices such as those disclosed herein.

In various embodiments, the digital processing device includes an input device to receive information from a user. In various embodiments, the input device is a keyboard. In various embodiments, the input device is a pointing device including, by way of non-limiting examples, a mouse, trackball, track pad, joystick, game controller, or stylus. In various embodiments, the input device is a touch screen or a multi-touch screen. In various embodiments, the input device is a microphone to capture voice or other sound input. In various embodiments, the input device is a video camera or other sensor to capture motion or visual input. In various embodiments, the input device is a Kinect, Leap Motion, or the like. In various embodiments, the input device is a combination of devices such as those disclosed herein.

### Non-Transitory Computer Readable Storage Medium

In various embodiments, and as stated above, the systems and methods disclosed herein can include, and the methods herein can be run on, one or more non-transitory computer readable storage media encoded with a program including instructions executable by the operating system of an optionally networked digital processing device. In various embodiments, a computer readable storage medium is a tangible component of a digital processing device. In various embodiments, a computer readable storage medium is optionally removable from a digital processing device. In various embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In various embodiments, the program and instructions are permanently, substantially permanently, semi-permanently, or non-transitorily encoded on the media.

### Computer Program

In various embodiments, the systems and methods disclosed herein can include at least one computer program, or use at least one computer program. A computer program includes a sequence of instructions, executable in the digital processing device's CPU, written to perform a specified task. Computer readable instructions may be implemented as program modules, such as functions, objects, Application Programming Interfaces (APis), data structures, and the like, that perform particular tasks or implement particular abstract data types. Those of ordinary skill in the art will recognize that a computer program may be written in various versions of various languages.

The functionality of the computer readable instructions may be combined or distributed as desired in various environments. In various embodiments, a computer program comprises one sequence of instructions. In various embodiments, a computer program comprises a plurality of sequences of instructions. In various embodiments, a computer program is provided from one location. In various embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In various embodiments, a computer program includes a web application. Those of ordinary skill in the art will recognize that a web application, in various embodiments, utilizes one or more software frameworks and one or more database systems. In various embodiments, a web application is created upon a software framework such as Microsoft®.NET or Ruby on Rails (RoR). In various embodiments, a web application utilizes one or more database systems including, by way of non-limiting examples, relational, non-relational, object oriented, associative, and XML database systems. In various embodiments, suitable relational database systems include, by way of non-limiting examples, Microsoft® SQL Server, mySQL™, and Oracle®. Those of ordinary skill in the art will also recognize that a web application, in various embodiments, is written in one or more versions of one or more languages. A web application may be written in one or more markup languages, presentation definition languages, client-side scripting languages, server-side coding languages, data-base query languages, or combinations thereof. In various embodiments, a web application is written to some extent in a markup language such as Hypertext Markup Language (HTML), Extensible Hypertext Markup Language (XHTML), or extensible Markup Language (XML). In various embodiments, a web application is written to some extent in a presentation definition language such as Cascading Style Sheets (CSS). In various embodiments, a web application is written to some extent in a client-side scripting language such as Asynchronous Javascript and XML (AJAX), Flash® ActionScript, JavaScript, or Silverlight®. In various embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tel, Smalltalk, WebDNA®, or Groovy. In various embodiments, a web application is written to some extent in a database query language such as Structured Query Language (SQL). In various embodiments, a web application integrates enterprise server products such as IBM® Lotus Domino®. In various embodiments, a web application includes a media player element. In various embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

### Mobile Application

In various embodiments, a computer program includes a mobile application provided to a mobile digital processing device. In various embodiments, the mobile application is provided to a mobile digital processing device at the time it is manufactured. In various embodiments, the mobile application is provided to a mobile digital processing device via the computer network described herein.

A mobile application can be created by techniques known to those of ordinary skill in the art using hardware, languages, and development environments known to the art. Those of ordinary skill in the art will recognize that mobile applications can be written in several languages. Suitable programming languages include, by way of non-limiting examples, C, C++, C#, Objective-C, Java™, JavaScript, Pascal, Object Pascal, Rust, Python™, Ruby, Vβ.NET, WML, and XHTML/HTML with or without CSS, or combinations thereof.

Suitable mobile application development environments are available from several sources. Commercially available development environments include, by way of non-limiting examples, AirplaySDK, alcheMo, Appcelera-tor®, Celsius, Bedrock, Flash Lite, .NET Compact Frame-work, Rhomobile, and WorkLight Mobile Platform. Other development environments are available without cost including, by way of non-limiting examples, Lazarus, Mobi-Flex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of ordinary skill in the art will recognize that several commercial forums are available for distribution of mobile applications including, by way of non-limiting examples, Apple® App Store, Google® Play, Chrome WebStore, BlackBerry® App World, App Store for Palm devices, App Catalog for webOS, Windows® Marketplace for Mobile, Ovi Store for Nokia® devices, Samsung® Apps, and Nin-tendo DSi Shop.

### Standalone Application

In various embodiments, a computer program includes a standalone application, which is a program that is run as an independent computer process, not an add-on to an existing process, e.g., not a plug-in. Those of ordinary skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and Vβ.NET, or combinations thereof. Compilation is often per-formed, at least in part, to create an executable program. In various embodiments, a computer program includes one or more executable complied applications.

### Web Browser Plug-in

In various embodiments, the computer program includes a web browser plug-in (e.g., extension, etc.). In computing, a plug-in is one or more software components that add specific functionality to a larger software application. Makers of software applications support plug-ins to enable third-party developers to create abilities, which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of ordinary skill in the art will be familiar with several web browser plug-ins including, Adobe® Flash® Player, Microsoft® Silver-light®, and Apple® QuickTime®. In various embodiments, the toolbar comprises one or more web browser extensions, add-ins, or add-ons. In various embodiments, the toolbar comprises one or more explorer bars, tool bands, or desk bands.

Those of ordinary skill in the art will recognize that several plug-in frame works are available that enable development of plug-ins in various programming languages, including, by way of non-limiting examples, C++, Delphi, Java™, PHP, Python™, and Vβ. NET, or combinations thereof.

Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Fire-fox®, Google® Chrome, Apple® Safari®, Opera Soft-ware® Opera®, and KDE Konqueror. In various embodiments, the web browser is a mobile web browser. Mobile web browsers (also called microbrowsers, mini-browsers, and wireless browsers) are designed for use on mobile digital processing devices including, by way of non-limiting examples, handheld computers, tablet computers, netbook computers, subnotebook computers, smartphones, and personal digital assistants (PDAs). Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony PSP™ browser.

### Software Modules

In various embodiments, the systems and methods disclosed herein include a software, server and/or database modules, or incorporate use of the same in methods according to various embodiments disclosed herein. Software modules can be created by techniques known to those of ordinary skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In various embodiments, software modules are in one computer program or application. In various embodiments, software modules are in more than one computer program or application. In various embodiments, software modules are hosted on one machine. In various embodiments, software modules are hosted on more than one machine. In various embodiments, software modules are hosted on cloud computing platforms. In various embodiments, software modules are hosted on one or more machines in one location. In various embodiments, software modules are hosted on one or more machines in more than one location.

### Databases

In various embodiments, the systems and methods disclosed herein include one or more databases, or incorporate use of the same in methods according to various embodiments disclosed herein. Those of ordinary skill in the art will recognize that many databases are suitable for storage and retrieval of user, query, token, and result information. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In various embodiments, a database is internet-based. In further Web. Suitable web browsers include, by way of non-limiting examples, Microsoft® Internet Explorer®, Mozilla® Fire-fox®, Google® Chrome, Apple® Safari®, Opera Soft-ware® Opera®, and KDE Konqueror. In various embodiments, the web browser is a mobile web browser. Mobile web browsers (also called microbrowsers, mini-browsers, and wireless browsers) are designed for use on mobile digital processing devices including, by way of non-limiting examples, handheld computers, tablet computers, netbook computers, subnotebook computers, smartphones, and personal digital assistants (PDAs). Suitable mobile web browsers include, by way of non-limiting examples, Google® Android® browser, RIM Blackberry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony PSP™ browser.

In various embodiments, a database is web-based. In various embodiments, a database is cloud computing-based. In other embodiments, a database is based on one or more local computer storage devices.

### Data Security

In various embodiments, the systems and methods disclosed herein include one or features to prevent unauthorized access. The security measures can, for example, secure a user's data. In various embodiments, data is encrypted. In various embodiments, access to the system requires multi-factor authentication and access control layer. In various embodiments, access to the system requires two-step authentication (e.g., web-based interface). In various embodiments, two-step authentication requires a user to input an access code sent to a user's e-mail or cell phone in addition to a username and password. In some instances, a user is locked out of an account after failing to input a proper username and password. The systems and methods disclosed herein can, in various embodiments, also include a mechanism for protecting the anonymity of users' genomes and of their searches across any genomes.

### ADDITIONAL CONSIDERATIONS

Any headers and/or subheaders between sections and subsections of this document are included solely for the purpose of improving readability and do not imply that features cannot be combined across sections and subsection. Accordingly, sections and subsections do not describe separate embodiments.

While the present teachings are described in conjunction with various embodiments, it is not intended that the present teachings be limited to such embodiments. On the contrary, the present teachings encompass various alternatives, modifications, and equivalents, as will be appreciated by those of skill in the art. The present description provides preferred exemplary embodiments, and is not intended to limit the scope, applicability, or configuration of the disclosure. Rather, the present description of the preferred exemplary embodiments will provide those skilled in the art with an enabling description for implementing various embodiments.

It is understood that various changes may be made in the function and arrangement of elements without departing from the spirit and scope as set forth in the appended claims. Thus, such modifications and variations are considered to be within the scope set forth in the appended claims. Further, the terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed.

In describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

Specific details are given in the present description to provide an understanding of the embodiments. However, it is understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

1. A computer-implemented method for visualizing cellular data, the method comprising:
receiving, by a processor, a data set comprising cellular data, wherein the cellular data includes at least one of an antigen binding specificity value associated with a first cell receptor and a unique molecular identifier (UMI) associated with the first cell receptor; and
presenting an end user with a visualization tool, wherein the visualization tool provides a dynamic display of the data set configured for analysis of the cellular data by:
generating, by the processor, a first visual sequence from the data set, wherein the first visual sequence includes a set of first indicia corresponding to a first chain of the first cell receptor and a set of second indicia corresponding to a second chain of the first cell receptor, wherein the set of first indicia is adjacent to the set of second indicia,
displaying at least a portion of the first visual sequence, and
displaying, in response to a first user interaction with the visualization tool, the antigen binding specificity value or the UMI associated with the first cell receptor in conjunction with the portion of the first visual sequence.

2. The computer-implemented method of claim 1, further comprising:
generating, by the processor, a second visual sequence from the data set, wherein the second visual sequence includes a set of third indicia corresponding to a third chain of a second cell receptor and a set of fourth indicia corresponding to a fourth chain of a second cell receptor, wherein the set of third indicia is adjacent to the set of fourth indicia; and
displaying at least a portion of the second visual sequence,
wherein the portion of the first visual sequence and the portion of the second visual sequence are displayed in parallel rows.

3. (canceled)

4. The computer-implemented method of claim 1, further comprising:
generating, by the processor, a plurality of visual sequences including the first visual sequence;
displaying the plurality of visual sequences, each in its own discrete row;
generating, by the processor, a table of information from the data set;
displaying the table of information, wherein the table of information includes genetic information for at least one of identified clonotypes and cell receptors belonging to a clonotype corresponding to the plurality of visual sequences;
receiving a first user selection of the first visual sequence;
receiving a second user selection of a third visual sequence of the plurality of visual sequences; and
modifying, by the processor, the table of information to generate a modified table of information, wherein information corresponding to the plurality of visual sequences is removed from the modified table of information except for information corresponding to the first visual sequence and the third visual sequence.

5. The computer-implemented method of claim 4, further comprising:
generating, by the processor in response to a second user interaction with the visualization tool, a file including the information corresponding to the first visual sequence and the third visual sequence.

6. The computer-implemented method of claim 1, wherein the cellular data includes T-Cell data, wherein the first chain is an alpha chain and the second chain is a beta chain.

7. (canceled)

8. The computer-implemented method of claim 1, wherein the cellular data includes B-Cell data, wherein the first chain is a heavy chain and the second chain is a light chain.

9. (canceled)

10. The computer-implemented method of claim 1, further comprising:
receiving, by the processor, a selection of a filter, wherein the filter is selected from a group consisting of: UMI Counts/antigen, antigen specificity, barcode, number of barcodes representing a clonotype, CDR3 amino acid sequence, CDR3 bases, gene name, isotype, cluster, amino acid % mutation, nucleotide % mutation, iNKT/MAIT, and combinations thereof;
modifying, by the processor, the first visual sequence based on the filter, to generate a modified first visual sequence that is different from the first visual sequence; and
displaying the modified first visual sequence.

11. The computer-implemented method of claim 10, further comprising:
generating, by the processor, a first table of information from the data set;
displaying the first table of information;
modifying, by the processor, the first table of information based on the filter, to generate a modified first table of information that is different from the first table of information; and
displaying the modified first table of information,
wherein at least one of the first table of information and the modified first table comprises genetic information for identified clonotypes or cell receptors belonging to a clonotype corresponding to at least one of the first visual sequence and the modified first visual sequence, wherein the genetic information is selected from a group consisting of V-gene, D-gene, J-gene, CDR sequence, and combinations thereof.

12. (canceled)

13. The computer-implemented method of claim 10, wherein the filter comprises multiple properties of VDJ sequences for heavy and light chains.

14. A computer program product comprising a computer-readable storage medium having program instructions embodied therewith, the program instructions executable by a processor to cause the processor to perform a method comprising:
receiving a data set comprising cellular data, wherein the cellular data includes at least one of an antigen binding specificity value associated with a first cell receptor and a unique molecular identifier (UMI) associated with the first cell receptor; and
presenting an end user with a visualization tool, wherein the visualization tool provides a dynamic display of the data set configured for analysis of the cellular data by:
generating a first visual sequence from the data set, wherein the first visual sequence includes a set of first indicia corresponding to a first chain of the first cell receptor and a set of second indicia corresponding to a second chain of the first cell receptor, wherein the set of first indicia is adjacent to the set of second indicia,
displaying at least a portion of the first visual sequence, and
displaying, in response to a first user interaction with the visualization tool, the antigen binding specificity value or the UMI associated with the first cell receptor in conjunction with the portion of the first visual sequence.

15-41. (canceled)

42. A computer implemented method for visualizing cellular data, the method comprising:
receiving, by a processor, a data set comprising cellular data;
receiving, by the processor, a selection of a filter, wherein the filter is selected from the group consisting of UMI Counts/antigen, antigen specificity, barcode, number of barcodes representing a clonotype, CDR3 amino acid sequence, CDR3 bases, gene name, isotype, cluster, amino acid % mutation, nucleotide % mutation, iNKT/MAIT, and combinations thereof; and
presenting an end user with a visualization tool, wherein the visualization tool provides a dynamic display of the data set configured for analysis of the cellular data by:
generating, by the processor, a first plot from the data set;
displaying the first plot;
modifying, by the processor, the first plot based on the filter, to generate a modified first plot that is different from the first plot; and
displaying the modified first plot.

43. The computer implemented method of claim 42, wherein the cellular data includes T-Cell data.

44. The computer implemented method of claim 42, wherein the cellular data includes B-Cell data.

45. The computer implemented method of claim 42, wherein at least one of the first plot and the modified first plot comprises a clonotype distribution plot.

46. The computer implemented method of claim 42, further comprising:
generating, by the processor, a first table of information from the data set;
displaying the first table of information; and
modifying, by the processor, the first table based on the filter, to generate a modified first table of information that is different from the first table of information, and displaying the modified first table of information,
wherein at least one of the first table of information and the modified first table of information comprises genetic information for identified clonotypes or cells belonging to a clonotype corresponding to the at least one of the first plot or the modified first plot, wherein the genetic information is selected from a group consisting of V-gene, D-gene, J-gene, CDR sequence, and combinations thereof.

47. (canceled)

48. The computer implemented method of claim 42, wherein the filter comprises multiple properties of VDJ sequences for heavy and light chains.

49. The computer implemented method of claim 42, wherein the filter is selected from the group consisting of UMI Counts/antigen, antigen specificity, barcode, number of barcodes representing a clonotype, isotype, cluster, amino acid % mutation, nucleotide % mutation, iNKT/MAIT information, and combinations thereof.

50. The computer implemented method of claim 42, wherein the first plot comprises a plurality of indicia, wherein each indicia represents a cell.

51. The computer implemented method of claim 50, wherein the modified first plot includes modified indicia when a cell, subclonotype, or clonotype corresponding to that indicia does not pass a criteria of the filter.

52. The computer implemented method of claim 51, wherein indicia have a first color when unmodified and a second color when modified.

53-65. (canceled)
